(19) United States
(12) Patent Application Publication
(10) Pub. No.: US 2017/0157301 A1
(43) Pub. Date: Jun. 8, 2017

(54) LEOLIGIN DERIVATIVES AS SMOOTH MUSCLE CELL PROLIFERATION INHIBITORS

(71) Applicants: TECHNISCHE UNIVERSITAET WIEN, Wien (AT); UNIVERSITAET INNSBRUCK, Innsbruck (AT)

(72) Inventors: Marko MIHOVILOVIC, Perchtoldsdorf (AT); Thomas LINDER, Wien (AT); Verena M. DIRSCH, Wien (AT); Atanas ATANASOV, Wien (AT); David BERNHARD, Woergl (AT); Herrmann STUPPNER, Goetzens (AT); Stefan SCHWAIGER, Innsbruck (AT)

(73) Assignees: TECHNISCHE UNIVERSITÄET WIEN, Wien (AT); UNIVERSITAET INNSBRUCK, Innsbruck (AT)

(21) Appl. No.: 15/322,107

(22) PCT Filed: Jun. 24, 2015
(86) PCT No.: PCT/AT2015/050160

§ 371 Date: Dec. 24, 2016

(30) Foreign Application Priority Data
Jun. 24, 2014 (AT) A 500/2014
Sep. 1, 2014 (AT) A 672/2014

(51) Int. Cl. A61L 31/16; C07D 307/16; C07D 405/06; C07D 307/12

(52) U.S. Cl. CPC A61L 31/16; C07D 307/12; C07D 307/16; C07D 405/06; A61L 2300/416

(57) ABSTRACT

A compound of formula (II), and a method of preparation thereof, for use as a smooth muscle cell (SMC) proliferation-inhibiting drug:

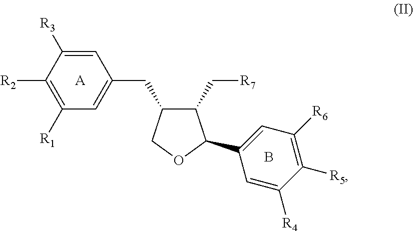

wherein: R₁ to R₆ are —H, —F, —CH₃, —CF₃, —CF₂CH₃, —OCH₃, —COCH₃, —C₄H₉, —COOC₂H₅, or —C₆H₅, or two vicinal residues from R₁ to R₆ form a saturated or unsaturated carbocyclic ring together with the two carbon atoms to which they are attached; R₇ is OH, allyloxy, propargyl-oxy, 2,2-dimethylpropanoyloxy (pivaloyloxy), butanoyloxy, 3-methylbutanoyloxy, 2-buten-oyloxy, 2-methyl-2-butenoyloxy, 3-methyl-2-butenoyloxy, isopentanoyloxy, 2-ethylbutanoyl-oxy, 3,3-dimethylbutanoyloxy, cyclopropylcarbonyloxy, cyclobutylcarbonyloxy, cyclo-pentylcarbonyloxy, cyclopentenylcarbonyloxy, cyclohexylcarbonyloxy, cyclo-hexenylcarbonyloxy, adamantylethanoyloxy, 3-phenylpropenoyloxy (cinnamyloyloxy), 2-methylbenzoyloxy, or naphthoyloxy; wherein, in ring A and/or B, one or more carbon ring atoms are optionally replaced by heteroatoms; wherein the compounds of formula (II) are obtained by combining the residues R₁ to R₇ and which inhibit SMC proliferation at least 50% more effectively than EC proliferation.

The present invention relates to Leoligin derivatives for use as proliferative inhibitors of smooth muscle cells, and to a novel method for synthesizing these compounds.

### PRIOR ART

Hyperplasia, i.e. the enlargement of tissues or organs caused by increased cell division, is a common phenomenon causing numerous disease states. Intimal hyperplasia which describes a thickening of the Tunica intima, the innermost layer of blood vessels facing the blood stream, frequently appearing after vascular surgery or percutaneous catheter intervention, is a major cause of narrowed blood vessels (stenosis) frequently resulting in serious consequences, e.g., coronary heart disease.

Actually, the reasons for intimal hyperplasia are not related to an increased cell proliferation of cells of the tunica intima, but rather to infiltration, followed by proliferation of smooth muscle cells (SMCs) originating in tunica media. Together with the incorporation of lipids, due to tissue reconstruction, intimal hyperplasia constitutes the basis for producing atherosclerotic plaques. Rupturing plaques lead to a formation of thrombi. Thrombotic scale-off and consequent thrombotic embolism, e.g., of coronary blood vessels, is one of the major causes for cardiac infarction and stroke.

A common method for treating intimal hyperplasia, as well as arteriosclerosis and atherosclerosis which may likewise lead to the occurrence of stenosis, and combinations thereof, such as vein graft disease, i.e. the joint occurrence of intimal hyperplasia and arteriosclerosis, comprises enlarging the vessel at the affected location using a catheter, and followed by positioning a stent, in order to prevent the risk of restenosis, i.e. of vascular re-narrowing. The respective mechanisms resemble the original processes, but they could also be induced or altered by the stent itself.

To prevent restenosis, it was proposed to coat the stent with a drug which inhibits cell proliferation, especially that of SMCs, prior to implantation. To that end, amongst others, Lariciresinol, a lignan occurring, for example, in sesame seeds and cabbage plants, and its (+) isomer, which is presented below, as well as a methoxy derivative thereof are disclosed (see g, IF 10 2004 046 244)

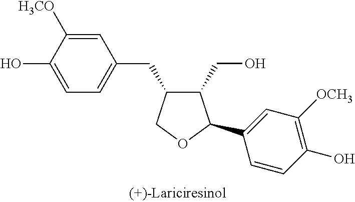 [SMILES: COc1cc(C[C@H]2CO[C@H](c3ccc(O)c(OC)c3)[C@H]2CO)ccc1O]

WO 2010/007169 A1 discloses several derivatives of (+) Lariciresinol, in particular (+)-Leoligin, i.e., (Z)-2-methyl-2-butenoic acid ((2S,3R,4R)-4-(3,4-dimethoxybenzyl)-2-(3,4-dimethoxyphenyl)tetrahydrofuran-3-yl)methyl ester:

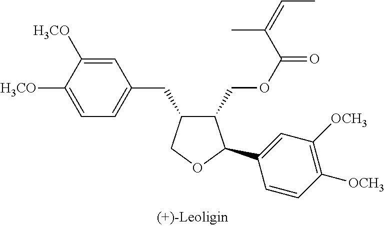 [SMILES: C/C=C(/C)C(=O)OC[C@H]1[C@@H](Cc2ccc(OC)c(OC)c2)CO[C@@H]1c1ccc(OC)c(OC)c1]

as well as its derivatives which are each substituted with 5-methoxy and 5,5′-dimethoxy, respectively, at both phenyl rings as effective inhibitors for the proliferation of smooth muscle tissues, both methoxy derivatives eclipsing the activity shown by Leoligin. Each of the three compounds which were extracted from edelweiss roots (Leontopodium alpinium Cass.) showed a markedly increased inhibitory activity (expressed as IC₅₀ values) compared to Lariciresinol.

WO 2010/007169 A1 claims anti-proliferative activity for compounds of the following formula (I), preferably those having the stereochemistry stated in formula (Ia), corresponding to that of (+)-Leoligin and (+)-Lariciresinol:

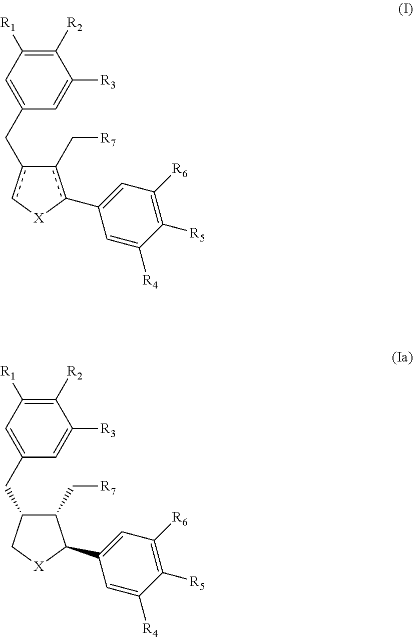

wherein: residues R₁ to R₆ are independently selected from H, OH, halogen, alkyl, and alkoxy, while R₇ is selected from the following residues: —OR₈, —N(R₈′)R₈, —SR₈, —C(O)R₈, —OC(O)R₉, —C(O)OR₉, —N(R₉′)C(O)R₉, —C(O)N(R₉′)R₉, and —S(O)R₉, wherein R₈ and R₉ are selected from alkyl and alkenyl, and R₈′ and R₉′ are selected from hydrogen, alkyl and alkenyl, which, in turn, may further be substituted with OH, halogen, or alkoxy, and X may be O, S, C(R₁₀)R₁₀ or NR₁₀, wherein R₁₀ is H, alkyl, or alkenyl.

FIG. 8 of WO 2010/007169 A1 depicts a reaction scheme based on the synthesis of furolignans which had already been disclosed by Roy et al., J. Org. Chem. 2002(67), 3242-3248, for the synthesis of Leoligin as well as derivatives thereof, with a carboxylic acid other than angelic acid in residue R₇. This synthesis generally comprises the separate preparation of the two phenyl moieties that are each substituted with two methoxy groups and a respective linker residue, combining both linker residues into a single linker moiety through an etherification reaction, and subsequent cyclization to form a corresponding tetrahydrofuran, in order to obtain Dimethyllariciresinol which finally is to be esterified into Leoligin using angelic acid, or into derivatives with various residues R₇ using an alternative carboxylic acid. FIG. 1 herein depicts this scheme.

Based on commercially available dimethoxycinnamic acid (dimethylcaffeic acid), reducing it to dimethoxycinnamyl alcohol and converting it into dimethoxycinnamyl bromide, the left one of both dimethoxy phenyl residues of formula (II) is thereby provided (first line in FIG. 1), and based on commercially available dimethoxybenzaldehyde (veratrumaldehyde), reacting the same with vinyl magnesium bromide to obtain racemic α-vinyl benzyl alcohol and oxidizing the same using m-chloro-perbenzoic acid to give the epoxide (α-oxiranyl benzyl alcohol) (see second line in FIG. 2), the residue on the right side is provided.

Subsequently, both of them are linked via an etherification to form quad-methoxy-substituted cinnamyl α-oxiranylbenzyl ether which is then radically cyclized using titanocene dichloride (dichlorobis(cyclopentadienyl)titanium(IV), Cp₂TiCl₂), thereby obtaining dimethyl lariciresinol as a 5:1 mixture of isomers, according to Roy et al. and WO 2010/007169 A1. Using preparative thin layer chromatography, the desired isomer is separated and is subjected to Steglich esterification using a suitable residue R₇ to give the corresponding carboxylic acid, N,N′-dicyclohexylcarbodiimide, DCC, and 4-dimethylaminopyridine, DMAP, to give the desired derivative of Leoligin.

This synthetic pathway exhibits the following disadvantages:
When preparing derivates of Leoligin having different substituents R₁ to R₆, at least one new intermediate product must be prepared for each individual combination of R₁ to R₆, which either results from the appropriately substituted benzaldehyde or the appropriately substituted cinnamic acid.
In doing so, moreover, the selection of substituents is limited to those being able to resist the initial reduction with LiAlH₄, so that particular compounds other than those taught in WO 2010/007169 A1 are precluded from the outset.
Further, in accordance with the synthesis pathway disclosed in Roy et al., racemates will be obtained instead of optically pure isomers, as is evident even from the title of Roy et al.: Disclosed is a “Short and Stereoselective Total Synthesis of Furano Lignans”, including (±)-Lariciresinol and its dimethyl ether. Therefore, actually, 2 pairs of enantiomers are obtained in the abovementioned ratio of 5:1, so that 4 isomers are obtained in total. Thus, after separating the unwanted racemic isomer present in a 1:5 shortfall, there still remains a racemate which must be resolved, either before or after the final esterification into Leoligin or a derivative thereof, in order to isolate the desired (+)-enantiomer. This, of course, reduces the yield by 50% which particularly affects the amounts of reagents used therein, because the unwanted enantiomer has been dragged along during six or seven steps of synthesis, respectively.

In light of the foregoing, it is an object of the present invention to synthesize new derivatives of Leoligin which exhibit an improved proliferation-inhibitory activity compared to Leoligin, as well as to develop a method for producing the same which allows for a safer and more cost-efficient way of preparing these novel derivatives than the current state of the art would imply.

### DISCLOSURE OF THE INVENTION

In a first aspect, the present invention is fulfilling these objects by providing compounds of the following formula (II) for use as agents that selectively inhibit the proliferation of smooth muscle cells (SMCs) compared to endothelial cells (ECs).

The compounds of formula (II) that were synthesized by the inventors are partly consistent with the definition of formula (Ia) in WO 2010/007169 A1 (if O is X), in other parts, however, they constitute novel compounds which could be used as drugs for inhibiting SMCs both in vivo as well as to inhibit them ex vivo, for example, in in vitro assays and several (other) assay formats. Sure enough, it was surprisingly found by the inventors that some compounds of formula (II) were able to inhibit SMC proliferation to a notably higher extent than EC proliferation, as will be explained in more detail below, which finding is of central importance to the present invention.

More specifically, in a first aspect, the invention relates, on the one hand, to novel compounds of the following formula (II) for use as a smooth muscle cell (SMC) proliferation-inhibiting drug:

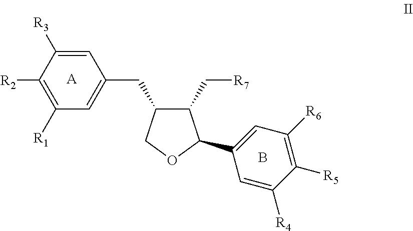

wherein:

R₁ to R₆ are selected from —H, —F, —CH₃, —CF₃, —CF₂CH₃, —OCH₃, —COCH₃, —C₄H₉, —COOC₂H₅, and —C₆H₅, or two vicinal residues selected from R₁ to R₆ are of such type and attached to each other that they form a saturated or unsaturated carbocyclic ring together with the two carbon atoms to which they are attached;

R₇ is selected from OH, propargyloxy, cyclopropylcarbonyloxy, cyclobutyl-carbonyloxy, cyclopentylcarbonyloxy, cyclopentenylcarbonyloxy, cyclohexylcarbonyl-oxy, cyclohexenylcarbonyloxy, adamantylethanoyloxy, 3-phenylpropenoyloxy (cinnamyloyloxy), 2-methylbenzoyloxy, and naphthoyloxy;

wherein, in ring A and/or in ring B, one or more ring carbon atoms may be replaced by heteroatoms;

wherein the compounds of formula (II) are selected from the following compounds that are obtained by appropriately combining residues R₁ to R₇:
((2S,3R,4R)-4-(4-(trifluoromethyl)benzyl)-2-(3,4,5-trimethoxyphenyl)tetrahydrofuran-3-yl)methanol (2760),
(Z)-2-methyl-2-butenoic acid ((2S,3R,4R)-2-(4-fluorophenyl)-4-(4-(trifluoromethyl)benzyl)tetrahydrofuran-3-yl)methyl ester (3016),
((2S,3R,4R)-2-(3,4-dimethoxyphenyl)-4-(4-methylbenzyl)tetrahydrofuran-3-yl)methanol (2754),
((2S,3R,4R)-4-benzyl-2-(3,4,5-trimethoxyphenyl)tetrahydrofuran-3-yl)methanol (2756),
(Z)-2-methyl-2-butenoic acid ((2S,3R,4R)-2-phenyl-4-(4-(trifluoromethyl)-benzyl)tetrahydrofuran-3-yl)methyl ester (3015),
(Z)-2-methyl-2-butenoic acid ((2S,3R,4R)-2-(3,4-dimethoxyphenyl)-4-(pyridine-2-ylmethyl)tetrahydrofuran-3-yl)methyl ester (2823),
((2S,3R,4R)-4-(biphenyl-4-ylmethyl)-2-(3,4,5-trimethoxyphenyl)tetrahydrofuran-3-yl) methanol (2767),
((2S,3R,4R)-4-(4-(1,1-difluoroethyl)benzyl)-2-(3,4-dimethoxyphenyl)tetrahydrofuran-3-yl)methanol (3013),
cyclobutanecarboxylic acid ((2S,3R,4R)-4-(3,4-dimethoxybenzyl)-2-(4-fluorophenyl)tetrahydrofuran-3-yl)methyl ester (2863),
cyclopentanecarboxylic acid ((2S,3R,4R)-4-(3,4-dimethoxybenzyl)-2-(3,4,5-trimethoxyphenyl)tetrahydrofuran-3-yl)methyl ester (2635),
cyclopropanecarboxylic acid ((2S,3R,4R)-4-(3,4-dimethoxybenzyl)-2-(3,4,5-trimethoxyphenyl)tetrahydrofuran-3-yl)methyl ester (2637),
((2S,3R,4R)-2-(3,4-dimethoxyphenyl)-4-(naphthalene-1-ylmethyl)tetrahydrofuran-3-yl)methanol (2821),
((2S,3R,4R)-4-(4-butylbenzyl)-2-(3,4-dimethoxyphenyl)tetrahydrofuran-3-yl)methanol (3008),
2-(adamantane-1-yl)acetic acid ((2S,3R,4R)-4-(3,4-dimethoxybenzyl)-2-(3,4-dimethoxyphenyl)tetrahydrofuran-3-yl)methyl ester (2745),
(Z)-2-methyl-2-butenoic acid ((2S,3R,4R)-4-(4-acetyl benzyl)-2-(3,4,5-trimethoxyphenyl)tetrahydrofuran-3-yl)methyl ester (2771),
4-(((3R,4R,5S)-4-((((Z)-2-methyl-2-butenoyl)oxy)methyl)-5-(3,4,5-trimethoxy-phenyl)tetrahydrofuran-3-yl)methyl)benzoic acid ethyl ester (2774),
cyclopentene-1-carboxylic acid ((2S,3R,4R)-4-(3,4-dimethoxybenzyl)-2-(3,4,5-trimethoxyphenyl)tetrahydrofuran-3-yl)methyl ester (2636),
cinnamic acid ((2S,3R,4R)-4-(3,4-dimethoxybenzyl)-2-(3,4-dimethoxyphenyl)-tetrahydrofuran-3-yl)methyl ester (2739),
cyclopentanecarboxylic acid ((2S,3R,4R)-4-(4-(tert-butyl)benzyl)-2-(3,4-dimethoxyphenyl)tetrahydrofuran-3-yl)methyl ester (2867),
cyclohexanecarboxylic acid ((2S,3R,4R)-4-(3,4-dimethoxybenzyl)-2-phenyltetrahydrofuran-3-yl)methyl ester (2746),
(2S,3R,4R)-4-(3,4-dimethoxybenzyl)-2-(3,4-dimethoxyphenyl)-3-(propargyl-oxymethyl)tetrahydrofuran (2541),
((2S,3R,4R)-2-(3,4-dimethoxyphenyl)-4-(4-(trifluoromethyl)benzyl)tetrahydrofuran-3-yl)methanol (3005),
((2S,3R,4R)-4-(4-(tert-butyl)benzyl)-2-(3,4-dimethoxyphenyl)tetrahydrofuran-3-yl)methanol (2749),
cyclopentanecarboxylic acid ((2S,3R,4R)-4-(3,4-dimethoxybenzyl)-2-phenyl-tetrahydrofuran-3-yl)methyl ester (2742),
(Z)-2-methyl-2-butenoic acid ((2S,3R,4R)-4-(4-(1,1-difluoroethyl)benzyl)-2-(3,4-dimethoxyphenyl)tetrahydrofuran-3-yl)methyl ester (3029),
cyclopentanecarboxylic acid ((2S,3R,4R)-4-(3,4-dimethoxybenzyl)-2-(4-fluoro-phenyl)tetrahydrofuran-3-yl)methyl ester (2781),
4-(((3R,4R,5S)-5-(3,4-dimethoxyphenyl)-4-((((Z)-2-methyl-2-butenoyl)oxy)-methyl)tetrahydrofuran-3-yl)methyl)benzoic acid ethyl ester (2792),
benzoic acid ((2S,3R,4R)-4-(3,4-dimethoxybenzyl)-2-(3,4,5-trimethoxyphenyl)tetrahydrofuran-3-yl)methyl ester (2638), and
cyclohexanecarboxylic acid ((2S,3R,4R)-4-(3,4-dimethoxybenzyl)-2-(3,4,5-trimethoxyphenyl)tetrahydrofuran-3-yl)methyl ester (2633);
wherein the compounds of formula (II) selectively inhibit smooth muscle cell (SMC) proliferation to a higher extent than endothelial cell (EC) proliferation;

and, on the other hand, also to compounds of formula (II) which were prepared and tested by the inventors for the first time, but which fall within the definition of formula (Ia) of WO 2010/007169 A1 (if O is X), i.e. compounds of the following formula (II) for use as a smooth muscle cell (SMC) proliferation-inhibiting drug:

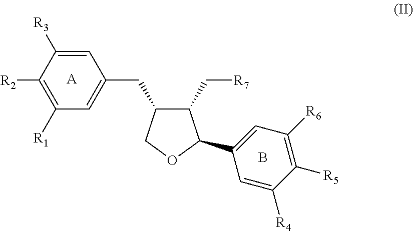

wherein:

R₁ to R₆ are selected from —H, —OH, halogen, alkyl, and alkoxy, and R₇ is selected from —OR₈, —N(R₈′)R₈, —SR₈, —C(O)R₈, —OC(O)R₉, —C(O)OR₉, —N(R₉′)C(O)R₉, —C(O)N(R₉′)R₉ and —S(O)R₉, wherein R₈ und R₉ are selected from alkyl and alkenyl, and R₈′ and R₉′ are selected from —H as well as alkyl and alkenyl, characterized in that:

a) in formula (II)

R₁ to R₆ are selected from —H, —F, —CH₃, —OCH₃ and —C₄H₉;

R₇ is selected from allyloxy, 2,2-dimethylpropanoyloxy (pivaloyloxy), butanoyl-oxy, 3-methylbutanoyloxy, 2-butenoyloxy, 2-methyl-2-butenoyloxy, 3-methyl-2-buten-oyloxy, isopentanoyloxy, 2-ethylbutanoyloxy and 3,3-dimethylbutanoyloxy;

wherein the compounds of formula (II) are selected from the following compounds that are obtained by appropriately combining the residues R₁ to R₇:
isobutyric acid ((2S,3R,4R)-4-(3,4-dimethoxybenzyl)-2-(3,4,5-trimethoxyphenyl)tetrahydrofuran-3-yl)methyl ester (2632),
(Z)-2-methyl-2-butenoic acid ((2S,3R,4R)-4-(4-butylbenzyl)-2-(3,4-dimethoxyphenyl)tetrahydrofuran-3-yl)methyl ester (3026),
(Z)-2-methyl-2-butenoic acid ((2S,3R,4R)-4-(4-(tert-butyl)benzyl)-2-(4-fluorophenyl)tetrahydrofuran-3-yl)methyl ester (3010),
(2S,3R,4R)-3-((allyloxy)methyl)-4-(3,4-dimethoxybenzyl)-2-(3,4-dimethoxyphenyl)tetrahydrofuran (2540),
3-methyl-2-butenoic acid ((2S,3R,4R)-4-(3,4-dimethoxybenzyl)-2-(3,4,5-trimethoxyphenyl)tetrahydrofuran-3-yl)methyl ester (2755),
3-methylbutyric acid ((2S,3R,4R)-4-(3,4-dimethoxybenzyl)-2-(4-fluorophenyl)-tetrahydrofuran-3-yl)methyl ester (2862),
(Z)-2-methyl-2-butenoic acid ((2S,3R,4R)-4-(4-fluorobenzyl)-2-(4-fluorophenyl)tetrahydrofuran-3-yl)methyl ester (2864),
butyric acid ((2S,3R,4R)-4-(3,4-dimethoxybenzyl)-2-(3,4,5-trimethoxyphenyl)-tetrahydrofuran-3-yl)methyl ester (2629),
(E)-2-methyl-2-butenoic acid ((2S,3R,4R)-4-(3,4-dimethoxybenzyl)-2-(3,4,5-trimethoxyphenyl)tetrahydrofuran-3-yl)methyl ester (2628),
(E)-2-butenoic acid ((2S,3R,4R)-4-(3,4-dimethoxybenzyl)-2-(3,4-dimethoxyphenyl)tetrahydrofuran-3-yl)methyl ester (2549),
(Z)-2-methyl-2-butenoic acid ((2S,3R,4R)-4-(4-(tert-butyl)benzyl)-2-(3, 4-dimethoxyphenyl)tetrahydrofuran-3-yl)methyl ester (2788),
(Z)-2-methyl-2-butenoic acid ((2S,3R,4R)-4-(4-(tert-butyl)benzyl)-2-(3,4,5-trimethoxyphenyl)tetrahydrofuran-3-yl)methyl ester (2765),
3-methyl-2-butenoic acid ((2S,3R,4R)-4-(3,4-dimethoxybenzyl)-2-(3,4-dimethoxyphenyl)tetrahydrofuran-3-yl)methyl ester (2738), and
2-ethylbutyric acid ((2S,3R,4R)-4-(3,4-dimethoxybenzyl)-2-(4-fluorophenyl)-tetrahydrofuran-3-yl)methyl ester (2856); and

b) the compounds of formula (II) selectively inhibit smooth muscle cell (SMC) proliferation to a higher extent than endothelial cell (EC) proliferation.

Surprisingly, it was found that each of the 43 compounds of formula (II) listed above selectively inhibit SMCs compared to ECs, as mentioned above, specifically to an extent which was up to 73 times higher than their activity against ECs. This is of particular surprise, because:

a) in total, 130 compounds were prepared by appropriately selecting substitutes R₁ to R₇, as described above, 2/3 of which did not exhibit this selectivity—and sometimes even showed inverse selectivity—and, above all,

b) Leoligin itself exhibits this inverse selectivity; as demonstrated using both the activity assays described herein (the efficiency ratio being 0.39, i.e. Leoligin inhibits ECs approximately 2.5 times more efficiently as SMCs), as and tests described by the state of the art.

For SMCs, the IC₅₀ for Leoligin was disclosed to be 54.5 μM, and at the same time the IC₅₀ for ECs was only 17.9 μM (A. Knolz, Master's Thesis, University of Innsbruck, Austria, 2008), giving a IC₅₀ ratio, serving as a measure for selective inhibition of SMCs compared to ECs, of 0.33 (and an inverse value, serving as a measure for selective inhibition of ECs compared to SMCs, of 3.0). These values almost precisely correspond to the ratio of 0.32 which was determined by the inventors for their least SMC-selective novel compound (2822; data not disclosed herein).

When selecting compounds showing selective inhibition of SMCs, as claimed herein, based on their activity ratios, because of relatively broad fluctuations which are caused by the calculation method, but also partly by the course of biological tests to be conducted, a threshold value was set to a ratio of 1.50 (see examples of embodiments below): each of the 43 compounds mentioned above thus inhibited SMCs with an efficiency which was 50% higher than the efficiency against ECs.

Preferably, however, the compound is selected from the following group comprising 32 compounds that inhibit SMCs at least twice as efficiently as ECs, i.e. that show an efficiency ratio of at least 2.0:
isobutyric acid ((2S,3R,4R)-4-(3,4-dimethoxybenzyl)-2-(3,4,5-trimethoxyphenyl)tetrahydrofuran-3-yl)methyl ester (2632),
(Z)-2-methyl-2-butenoic acid ((2S,3R,4R)-4-(4-butyl benzyl)-2-(3,4-dimethoxyphenyl)tetrahydrofuran-3-yl)methyl ester (3026),
((2S,3R,4R)-4-(4-(trifluoromethyl)benzyl)-2-(3,4,5-trimethoxyphenyl)tetrahydrofuran-3-yl)methanol (2760),
(Z)-2-methyl-2-butenoic acid ((2S,3R,4R)-4-(4-(tert-butyl)benzyl)-2-(4-fluorophenyl)tetrahydrofuran-3-yl)methyl ester (3010),
(2S,3R,4R)-3-((allyloxy)methyl)-4-(3,4-dimethoxybenzyl)-2-(3,4-dimethoxyphenyl)tetrahydrofuran (2540),
(Z)-2-methyl-2-butenoic acid ((2S,3R,4R)-2-(4-fluorophenyl)-4-(4-(trifluoromethyl)benzyl)tetrahydrofuran-3-yl)methyl ester (3016),
((2S,3R,4R)-2-(3,4-dimethoxyphenyl)-4-(4-methylbenzyl)tetrahydrofuran-3-yl)methanol (2754),
3-methyl-2-butenoic acid ((2S,3R,4R)-4-(3,4-dimethoxybenzyl)-2-(3,4,5-trimethoxyphenyl)tetrahydrofuran-3-yl)methyl ester (2755),
((2S,3R,4R)-4-benzyl-2-(3,4,5-trimethoxyphenyl)tetrahydrofuran-3-yl)methanol (2756),
(Z)-2-methyl-2-butenoic acid ((2S,3R,4R)-2-phenyl-4-(4-(trifluoromethyl)-benzyl)tetrahydrofuran-3-yl)methyl ester (3015),
3-methylbutenoic acid ((2S,3R,4R)-4-(3,4-dimethoxybenzyl)-2-(4-fluorophenyl)tetrahydrofuran-3-yl)methyl ester (2862),
(Z)-2-methyl-2-butenoic acid ((2S,3R,4R)-4-(4-fluorobenzyl)-2-(4-fluorophenyl)tetrahydrofuran-3-yl)methyl ester (2864),
butyric acid ((2S,3R,4R)-4-(3,4-dimethoxybenzyl)-2-(3,4,5-trimethoxyphenyl)-tetrahydrofuran-3-yl)methyl ester (2629),
(E)-2-methyl-2-butenoic acid ((2S,3R,4R)-4-(3,4-dimethoxybenzyl)-2-(3,4,5-trimethoxyphenyl)tetrahydrofuran-3-yl)methyl ester (2628),
(Z)-2-methyl-2-butenoic acid ((2S,3R,4R)-2-(3,4-dimethoxyphenyl)-4-(pyridine-2-ylmethyl)tetrahydrofuran-3-yl)methyl ester (2823),
(E)-2-butenoic acid ((2S,3R,4R)-4-(3,4-dimethoxybenzyl)-2-(3,4-dimethoxyphenyl)tetrahydrofuran-3-yl)methyl ester (2549),
((2S,3R,4R)-4-(biphenyl-4-yl-methyl)-2-(3,4,5-trimethoxyphenyl)tetrahydrofuran-3-yl)methanol (2767),
((2S,3R,4R)-4-(4-(1,1-difluoroethyl)benzyl)-2-(3,4-dimethoxyphenyl)tetrahydrofuran-3-yl)methanol (3013),
cyclobutanecarboxylic acid ((2S,3R,4R)-4-(3,4-dimethoxybenzyl)-2-(4-fluorophenyl)tetrahydrofuran-3-yl)methyl ester (2863),
cyclopentanecarboxylic acid ((2S,3R,4R)-4-(3,4-dimethoxybenzyl)-2-(3,4,5-trimethoxyphenyl)tetrahydrofuran-3-yl)methyl ester (2635),
cyclopropanecarboxylic acid ((2S,3R,4R)-4-(3,4-dimethoxybenzyl)-2-(3,4,5-trimethoxyphenyl)tetrahydrofuran-3-yl)methyl ester (2637),
((2S,3R,4R)-2-(3,4-dimethoxyphenyl)-4-(naphthalene-1-ylmethyl)tetrahydrofuran-3-yl)methanol (2821),
((2S,3R,4R)-4-(4-butylbenzyl)-2-(3,4-dimethoxyphenyl)tetrahydrofuran-3-yl)methanol (3008),
2-(adamantane-1-yl)acetic acid ((2S,3R,4R)-4-(3,4-dimethoxybenzyl)-2-(3,4-dimethoxyphenyl)tetrahydrofuran-3-yl)methyl ester (2745),
(Z)-2-methyl-2-butenoic acid ((2S,3R,4R)-4-(4-(tert-butyl)benzyl)-2-(3,4-dimethoxyphenyl)tetrahydrofuran-3-yl)methyl ester (2788),
(Z)-2-methyl-2-butenoic acid ((2S,3R,4R)-4-(4-acetyl benzyl)-2-(3,4,5-trimethoxyphenyl)tetrahydrofuran-3-yl)methyl ester (2771),
(Z)-2-methyl-2-butenoic acid ((2S,3R,4R)-4-(4-(tert-butyl)benzyl)-2-(3,4,5-trimethoxyphenyl)tetrahydrofuran-3-yl)methyl ester (2765),
3-methyl-2-butenoic acid ((2S,3R,4R)-4-(3,4-dimethoxybenzyl)-2-(3,4-dimethoxyphenyl)tetrahydrofuran-3-yl)methyl ester (2738),
4-(((3R,4R,5S)-4-((((Z)-2-methyl-2-butenoyl)oxy)methyl)-5-(3,4,5-trimethoxy-phenyl)tetrahydrofuran-3-yl)methyl)benzoic acid ethyl ester (2774),
cyclopentene-1-carboxylic acid ((2S,3R,4R)-4-(3,4-dimethoxybenzyl)-2-(3,4,5-trimethoxyphenyl)tetrahydrofuran-3-yl)methyl ester (2636),
cinnamic acid ((2S,3R,4R)-4-(3,4-dimethoxybenzyl)-2-(3,4-dimethoxyphenyl)-tetrahydrofuran-3-yl)methyl ester (2739), and
cyclopentanecarboxylic acid ((2S,3R,4R)-4-(4-(tert-butyl)benzyl)-2-(3,4-dimethoxyphenyl)tetrahydrofuran-3-yl)methyl ester (2867).

In particularly preferred embodiments, the compound is selected from the following group comprising 15 compounds which inhibit SMCs at least five times more efficiently than ECs, i.e. that show an efficiency ratio of at least 5.0:
isobutyric acid ((2S,3R,4R)-4-(3,4-dimethoxybenzyl)-2-(3,4,5-trimethoxyphenyl)tetrahydrofuran-3-yl)methyl ester (2632),
(Z)-2-methyl-2-butenoic acid ((2S,3R,4R)-4-(4-butyl benzyl)-2-(3,4-dimethoxyphenyl)tetrahydrofuran-3-yl)methyl ester (3026),
((2S,3R,4R)-4-(4-(trifluoromethyl)benzyl)-2-(3,4,5-trimethoxyphenyl)tetrahydrofuran-3-yl)methanol (2760),
(Z)-2-methyl-2-butenoic acid ((2S,3R,4R)-4-(4-(tert-butyl)benzyl)-2-(4-fluorophenyl)tetrahydrofuran-3-yl)methyl ester (3010),
(2S,3R,4R)-3-((allyloxy)methyl)-4-(3,4-dimethoxybenzyl)-2-(3,4-dimethoxyphenyl)tetrahydrofuran (2540),
(Z)-2-methyl-2-butenoic acid ((2S,3R,4R)-2-(4-fluorophenyl)-4-(4-(trifluoromethyl)benzyl)tetrahydrofuran-3-yl)methyl ester (3016),
((2S,3R,4R)-2-(3,4-dimethoxyphenyl)-4-(4-methylbenzyl)tetrahydrofuran-3-yl)methanol (2754),
3-methyl-2-butenoic acid ((2S,3R,4R)-4-(3,4-dimethoxybenzyl)-2-(3,4,5-trimethoxyphenyl)tetrahydrofuran-3-yl)methyl ester (2755),
((2S,3R,4R)-4-benzyl-2-(3,4,5-trimethoxyphenyl)tetrahydrofuran-3-yl)methanol (2756),
(Z)-2-methyl-2-butenoic acid ((2S,3R,4R)-2-phenyl-4-(4-(trifluoromethyl)-benzyl)tetrahydrofuran-3-yl)methyl ester (3015),
3-methylbutenoic acid ((2S,3R,4R)-4-(3,4-dimethoxybenzyl)-2-(4-fluorophenyl)tetrahydrofuran-3-yl)methyl ester (2862),
(Z)-2-methyl-2-butenoic acid ((2S,3R,4R)-4-(4-fluorobenzyl)-2-(4-fluorophenyl)tetrahydrofuran-3-yl)methyl ester (2864),
butyric acid ((2S,3R,4R)-4-(3,4-dimethoxybenzyl)-2-(3,4,5-trimethoxyphenyl)-tetrahydrofuran-3-yl)methyl ester (2629),
(E)-2-methyl-2-butenoic acid ((2S,3R,4R)-4-(3,4-dimethoxybenzyl)-2-(3,4,5-trimethoxyphenyl)tetrahydrofuran-3-yl)methyl ester (2628),
(Z)-2-methyl-2-butenoic acid ((2S,3R,4R)-2-(3,4-dimethoxyphenyl)-4-(pyridine-2-ylmethyl)tetrahydrofuran-3-yl)methyl ester (2823).

Due to the above-mentioned fluctuations, however, it cannot be precluded that additional compounds obtained by combining the above substituents in formula (II) that previously did not exceed the threshold value of 1.5 will be found to be equally active in the course of future activity assays. Obviously, such compounds should also be encompassed by the scope of the accompanying claims.

Thus, a preferable agent comprising the compound of formula (II) for selectively inhibiting smooth muscle cell (SMC) proliferation in comparison to endothelial cell (EC) proliferation is a drug meant for treating a human or animal patient in need of same. The use of the drug for treating or preventing hyperplasia, especially intimal hyperplasia, stenosis or restenosis is especially preferred.

To this end, the nature of the drug's formulation and administration is not particularly limited, as long as the efficiency of the compound of formula (II) is not compromised. Thus, the way of administration may for example be systemic or topical; e.g., oral, rectal, parenteral, intracisternal, intravaginal, intraperitoneal, intravenous, intra-arterial, intramuscular, intracardial, intrapulmonary, intravesical, intravitreal, subcutaneous, sublingual, intranasal, or transdermal using patches—depending on the region of the body to be treated for hyperplasia.

In preferred embodiments, the drug is for coating or impregnating a stent or other implant which is subsequently transplanted into the patient's body. The implant may be prepared artificially or may be any tissue, organ or part of an organ, especially a section of a vein, which is harvested from a patient and, once coated with the drug, is to be re-implanted into the patient.

An especially preferred use of the drug is for coating the above-mentioned, so-called “drug eluting”, i.e. drug-coated, stents which are implanted, for example, into a patient's blood vessel afflicted by stenosis in order to dilate the same.

Subsequently, the drug can efficiently prevent restenosis caused by smooth muscle cells growing into the stent's lumen. Due to the selective effect of compounds of the present invention, the healing process of the Tunica intima, i.e. of the monocellular layer consisting of endothelial cells, which is usually damaged when introducing the stent, is not or hardly inhibited.

Thus, in a second aspect, the invention provides a pharmaceutical composition suitable for such treatment regimes, which comprises a compound as previously defined as well as a pharmaceutically acceptable carrier or excipient, and optionally one or more adjuvants and/or one or more other active ingredients. Those skilled in the art of pharmaceutics will be able to readily develop formulations suitable for every respective purpose without undue experimentation, but via standard routine testing. Instructions therefor can be found in numerous pharmaceutical standard publications such as “Remington: Essentials of Pharmaceutics”, L. Felton (Ed.), Pharmaceutical Press, Gurnee, Ill., 2013.

In a third aspect, the invention relates to the use of a compound of formula (II) as previously defined herein for selectively inhibiting smooth muscle cell (SMC) proliferation compared to endothelial cell (EC) proliferation ex vivo or in vivo, respectively, for example in various assay formats.

In a fourth aspect, the present invention relates to a method for coating a body implant by applying a drug onto at least one surface of the implant, characterized in that a compound of formula (II) as previously defined herein is being applied as said drug.

Preferably, said implant is a drug-coated (“drug eluting”) stent which is coated with such a drug. Those skilled in the art are familiar with steps which are necessary for the achievement thereof, however they can also be found, for example, in “Handbook of Coronary Stents”, P. W. Serruys, B. Rensing, S. W. Serruys (Ed.), In-forma Healthcare (November 2001), or in “Handbook of Drug-eluting Stents”, P. W. Serruys and A. H. Gershlick (Ed.), Informa Healthcare (June 2005). Such a stent prepared according to the present invention constitutes a fifth aspect of the invention.

Finally, in a sixth aspect, the present invention provides a method for preparing said compounds of formula (II) as previously defined herein, characterized by the following reaction steps a) to h):

a) reacting a benzaldehyde of formula (I), substituted with the respective residues R₄ to R₆, with vinyl magnesium bromide, or an alternative vinyl nucleophile, to obtain the respective racemic α-vinyl benzyl alcohol rac-(2), which reaction is known per se:

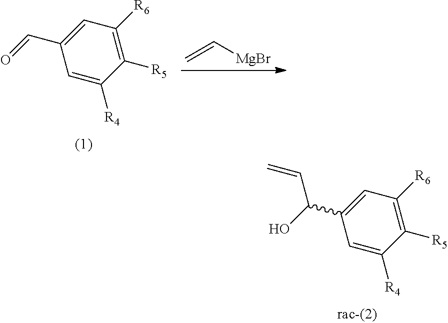

b) kinetic resolution of rac-(2) by means of enzymatic catalysis to obtain the (S)-enantiomer (S)-(2) with simultaneous formation of (R)—R₈-(2) as a by-product to be separated, wherein R₈ preferably is an acyl residue:

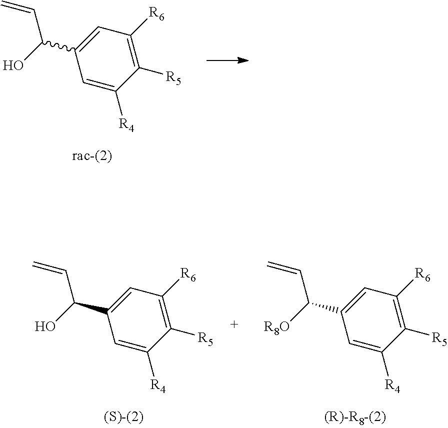

c) subsequently esterifying and oxidizing (S)-(2) in arbitrary order, i.e. either:

c1) esterification of (S)-(2) with propargyl bromide, or an alternative propargylating agent, to give the propargyl ether (3):

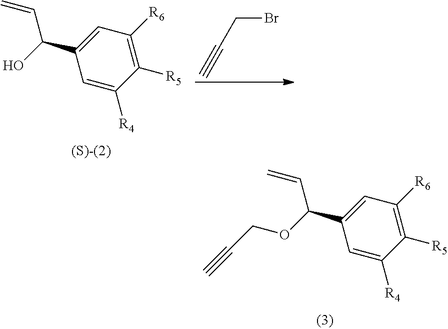

and subsequent oxidation of (3) to give the propargyl ether epoxide (5):

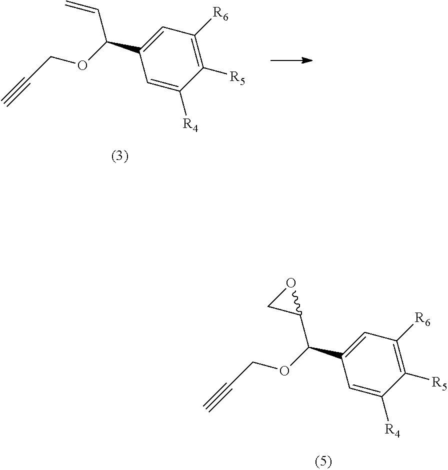

or, in reverse order:

c2) oxidizing (S)-(2) to give the epoxide (4):

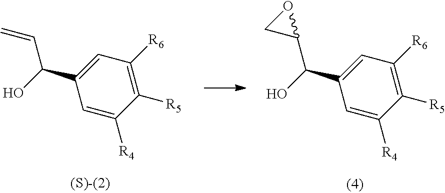

and subsequently performing esterification of (4) with propargyl bromide, or an alternative propargylating agent, to give the propargyl ether epoxide (5):

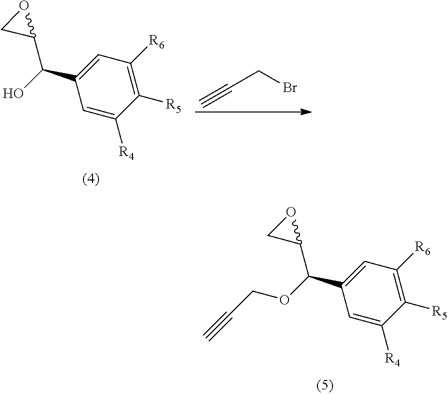

d) cyclization of (5) using a cyclization agent to give the tetrahydrofuranyl methanol (6):

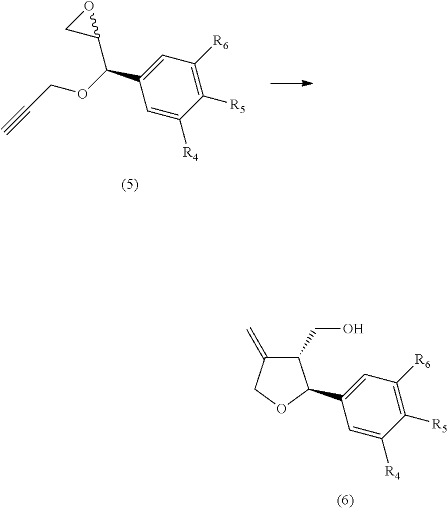

(e) protecting the alcohol functionality of (6) using the tert-butyl dimethyl silyl (TBDMS) or an alternative bulky protecting group to give the protected methanol (7):

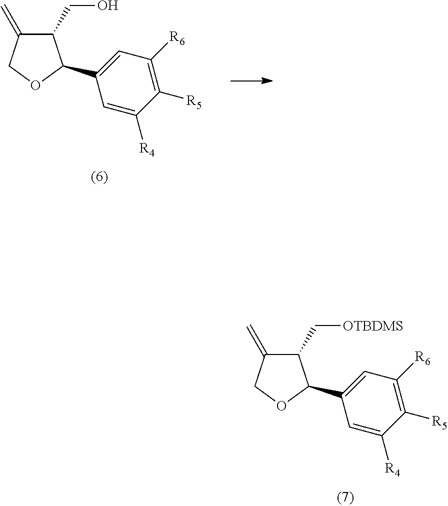

f) introducing a phenyl or 2-pyridyl residue (7b), substituted with the corresponding residues R₁ to R₃, by reaction with the exocyclic methylene group of (7) to give the protected product (8):

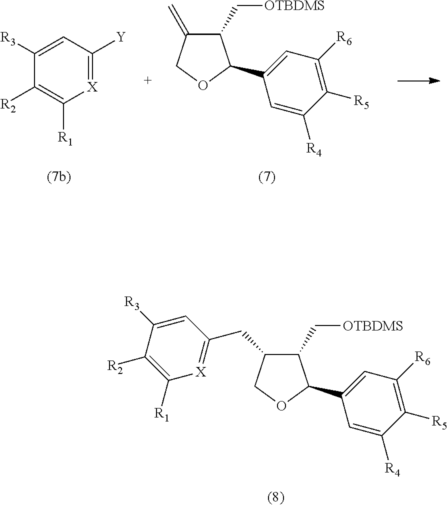

wherein, in each case, X is CH or N and Y is a leaving group;

g) cleaving the protecting group of (8) to give the free alcohol (9):

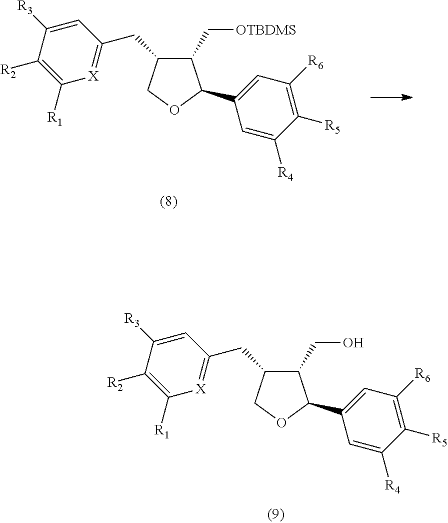

h) if R₇≠OH, simultaneously or subsequently etherifying or esterifying the free OH group, in order to obtain compound (10) comprising the corresponding residue R₇:

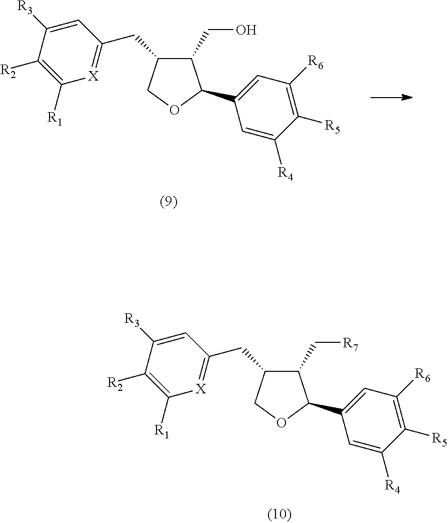

Compared to the preparation method disclosed in WO 2010/007169 and in Roy et al. (supra), this route of synthesis offers many significant benefits, especially the ones as outlined in the following in points 1) to 6):

1) A chiral resolution is already performed during the second synthesis step b) to obtain the desired (S) isomer of intermediate (2) containing the phenyl group in its appropriate spacial orientation, substituted with the residues R₄ to R₆, which, subsequently, will be located at position 2 of said tetrahydrofuran.

2) In contrast to WO 2010/007169 A1 and Roy et al. (supra), the cyclization of step d) is performed prior to the attachment of the aryl residue substituted with R₁ to R₃ to the cyclized tetrahydrofuran. This allows for a notably easier variation of substituents R₁ to R₇, because a cyclized and esterified intermediate (7) comprising a defined combination of R₄ to R₆ may be combined with different compounds (7b), each of them comprising, in turn, a particular combination of R₁ to R₃, to give a plurality of intermediates (8) by performing only one single step or very few individual steps.

Subsequently (after removing the protecting group), these intermediates may be reacted by means of a simple esterification reaction with a wide range of acid residues to yield one new derivative (10) of formula (II) each time. If, however, said OH protecting group and the ethanolic oxygen together are already forming the eventual substituent R₇, this may be omitted, because intermediate (8) will already correspond to formula (II). Thus, the present invention allows for a much easier preparation of a comprehensive library of derivatives of formula (II) to be tested for their efficacy.

3) Further, the cyclization of step d) between epoxide and alkynyl group to give an exocyclic methylene group does not yield a 5:1 mixture of racemic diastereomers, as the cyclization between epoxide and phenyl-substituted alkenyl group described in WO 2010/007169 A1 and Roy et al. (supra) does.

Furthermore, the cyclization performed in the method of the present invention has already been described by a working group lead by Subhas Chandra Roy: for an alkyl substituent at the position of tetrahydrofuran see G. Maiti and S. C. Roy, J. Chem. Soc., Perkin Trans. 1, 403-404 (1996) (abbr.: Maiti et al.), and for various substituents including said dimethoxy phenyl leading to Leoligin and dimethyl lariciresinol, respectively, see P. K. Mandal, G. Maiti, S. C. Roy, J. Org. Chem. 63, 2829-2834 (1998) (abbr.: Mandal et al.).

In both of the latter publications, diastereomeric ratios of 5:1, or even 6:1 in Mandal et al., are given for 2-alkyl substituents at the tetrahydrofurans formed in this process, while moieties with bulkier structures, e.g. (substituted) phenyl or naphthyl, only yielded the desired diastereomer having a 2,3-trans configuration of substituents at both chiral centers. Using phenyl substituted with R₄ to R₆ throughout all syntheses, the present inventors have confirmed this.

In addition to from the bulky structure of said substituent at position 2 of the tetrahydrofuran, the absence of a third chiral center at C4 also contributes to the stereoselectivity of the cyclization reaction. According to the present invention, the third chiral center at C4 is introduced into the molecule not before the subsequent step f).

4) By using a bulky protecting group in step e), stereoselectivity is increased dramatically during the subsequent functionalization of said exocyclic methylene group. Apart from the tert-butyl dimethyl silyl group preferably utilized as the protecting group, which provides many benefits including space requirements as well as low costs, chemical stability, and yet easy cleavability, other silyl protecting groups, such as triisopropyl silyl (TIPS) and tert-butyl diphenyl silyl (TBDPS), or, more generally, other hydroxyl protecting groups, such as acyclic, acetal, ketal, benzylic or alkyl OH protecting groups, may be used, too; however, they may lead to poorer results during the consecutive step f).

5) Not only, but especially when R₇ constitutes a substituent having a more or less bulky structure, an acid residue immediately leading to R₇ may be esterified with said free OH group instead of using a protecting group, thus shortening the synthesis by two steps as removal of the protecting group and re-esterification via steps g) and h) may be omitted.

In other words, the same also applies when said OH group is to be etherified instead of esterified in order to form R₇. Throughout the hitherto existing examples of the invention, the alcohol was solely etherified with allyl or propargyl, respectively, which are not bulky enough to develop a distinctive directing effect during the addition in step f), and which might furthermore undergo unwanted side reactions in step f). Using tert-butyl or (hetero-)aryl residues, for example, the etherification of step h) could be preponed to step e), and steps g) and h) could be omitted.

6) Functionalization of the exocyclic double bond in step f) is one major step and benefit of the method according to the present invention. As already mentioned above, a bulky OH protecting group (or a bulky ester or ether as the substituent R₇, respectively) increases stereoselectivity of this step. Utilizing the presence of said protecting group allows for various options of stereoselective functionalization: i) double bond pre-functionalization using a suitable reagent, the addition product of which being able to react with a suitable reaction partner in situ or after work-up, in order to introduce said aryl rest substituted with R₁ to R₃, such as double bond hydro-metallization followed by a coupling reaction; ii) double bond epoxidation followed by ring-opening using a suitable nucleophile, and followed by removal of the OH group hence formed; iii) functionalizing while retaining the double bond, e.g., by means of the Heck reaction or CH activation, especially sp2-sp2-CH activation followed by reduction; or iv) double bond functionalization upon simultaneous conversion into a single bond, e.g., by means of a reductive Heck reaction or direct functionalization, provided that the conversion into a single bond can be effected while keeping the desired stereochemistry.

Additional potential and preferred variants of the steps of the inventive method will be described in more detail hereinafter.

According to a preferred embodiment of the invention, in step b), Amano Lipase PS is used as the enzyme and vinyl acetate or isopropenyl acetate are used as its donor component, so that residue R₈ is an acetyl residue and (R)-acetyl-(2) is formed as a by-product.

Generally speaking, enzymatically catalyzed reactions are characterized by high stereoselectivity—a feature which is utilized by the present invention in step b), in order to determine the stereoselectivity of one out of three chiral centers at the subsequent tetrahydrofuran ring already during an early stage of synthesis.

Furthermore, as the undesired enantiomer is the reacting one, and the desired enantiomer is the remaining one, an enantiomeric excess, ee, of almost 100% can be achieved.

Preferably, Lipase PS from Amano Enzyme Inc. will be employed as the enzyme together with vinyl or isopropenyl acetate acting as a donor or co-substrate, since this has proven to give the highest yields. The lipase preparation comprises a lipolytic enzyme immobilized on diatomaceous earth, which selectively confers the acetyl group onto the (R)-isomer of rac-(2) in the presence of vinyl acetate, whereafter the thus formed (R)-acetyl-(2) may be easily separated from the desired alcohol (S)-(2), e.g., via flash chromatography, which is also possible with larger scale reaction mixtures (>50 g).

A an alternative to Lipase PS, also Lipase CALB-L (immobilized Candida antarctica Lipase B, commercially available as Novozymes 435) or other enzymes suitable for this purpose, especially other lipases, esterases, proteases, or acyl transferases, for example, which are known in the art could be used. According to literature, e.g., in Stambasky et al., J. Org. Chem. 73(22), 9148-9150 (2008), a corresponding esterification using Novozymes 435 is described.

As mentioned above, the following etherification of the benzyl alcohol to give the propargyl ether and oxidation of the vinyl group to give the epoxide may be performed in arbitrary order. Step c1) follows the order mentioned above, while in the alternative step c2), the intermediate (S)-(2) will first be oxidized and etherified thereafter.

In both cases, propagylation will be performed preferably using propargyl bromide, which is also described by Mandal et al. (supra), for example. Alternatively, other substituents conferring a positive charge to the propargyl residue, such as chloride, iodide, tosylate, or mesylate, as is generally known for Williamson etherifications, could also be used. Preparing the alkoxide of the benzyl alcohol (3) or (4), respectively, may be done by conventional means, for example, using strong bases, preferably NaH or KH, or even lithium diisopropylamide (LDA), butyl lithium (BuLi), or potassium tert-butanolate (KO-t-Bu, KTB).

In step c1), the oxidation, which is performed after the propargylation, will be conducted preferably using m-chloroperbenzoic acid as the oxidant, which is also described by Mandal et al. (supra), since this is a tested and comparably cost-effective reaction. Although this results in a diastereomeric mixture of both possible epoxides, the stereochemistry at the carbon atom in β-position relative to said phenyl residue is unimportant, because during the subsequent cyclization a trigonal-planar C radical will be formed at this position, and the desired 2,3-trans-selectivity will only be caused by the aryl residue at position 2.

According to the present invention, however, reverse order will be preferred, i.e. performing step c2), wherein the oxidation, which is carried out before propargylation, will be performed as a Sharpless epoxidation using (S,S)-diethyl tartrate, (−)-DET, as a co-reagent, tert-butyl hydroperoxide, TBHP, as the oxidant, and titanium(IV) isopropanolate, Ti(O-i-Pr)₄, as a catalyst, which leads to higher yields for electron-rich aromatics than an oxidation using m-chlorobenzoic acid. Again, subsequent propargylation is preferably carried out using propargyl bromide.

As is known from Mandal et al., titanocene dichloride (dichloro-bis-cyclo-pentadienyl) titanium(IV), Cp₂TiCl₂) is a preferred cyclization agent in the subsequent cyclization of the propargylated epoxide (5) performed in step d), since such a reaction, as mentioned above, will substantially lead to the production of the desired respective stereoisomer alone. Alternatively, albeit not preferably, also a trialkyl stannane together with azobisisobutyronitrile (AIBN) could be used as a free radical cyclization agent, if the epoxide is ring-opened via acid catalysis in the presence of bromide, or if a (formal) addition of hypobromous acid to the vinylic double bond is performed instead of epoxidation, in order to introduce a Br substituent in β-position.

As previously mentioned, if R₇≠OH, the OH group may be protected in step e) by esterification with the respective acyl residue—or by etherification with the respective alkyl or aryl residue —, that will form the respective residue R₇ together with the oxygen atom, whereafter it is possible to omit the following steps g) and h) in preferred embodiments. This is especially true for bulky residues, so that they might develop a directing effect during the subsequent addition to the exocyclic methylene group, in order to substantially obtain the desired stereoisomer alone.

According to preferred embodiments of the present invention, a Suzuki coupling is performed in step f), during which the exocyclic methylene group of (7) will first be stereoselectively pre-functionalized in an intermediary step f1) into intermediate (7a) using an organoborane, wherein a hydroboration is performed as a preferred embodiment of the above-mentioned hydrometallization, whereafter (7a) will be immediately reacted with compound (7b) in step f2), wherein X is a halogen or pseudo-halogen (e.g., triflate or nonaflate), in order to substitute the boryl residue with said phenyl or 2-pyridyl residue substituted with R₁ to R₃.

This pre-functionalization method includes the benefit that the desired stereoselectivity is solely induced by the protecting group and, thus, is not dependent on the steric requirement of the coupling partner (7b). Most notably, hydroboration—when used for pre-functionalization—constitutes a reliable and safe method of reacting said exocyclic double bond under mild conditions. What is more, also the subsequent Suzuki coupling is very insensitive to the electronic properties of compound (7b).

Mechanistically, the addition of borane is performed via a tetratomic transition state in which the B—H bond being broken is re-orienting itself into a position parallel to the C—C double bond, which for steric reasons is done in a regioselective way with the dialkylboryl part located above the distal carbon atom and the hydridic hydrogen located above the proximal carbon atom of the double bond. As a result, only intermediate (7a) having the respective stereoselectivity is formed. Regarding the 3,4-cis stereoselectivity of the reaction, the result will depend on the steric requirement of the OH protecting group, diastereoselectivities of up to 97:3 being achieved by the preferred embodiment using TBDMS. When less bulky protecting groups are used, the likelihood of an attack with a resulting trans configuration is increased, so that during addition, stereoisomeric mixtures in (sometimes notably) poorer ratios are obtained.

Therefore, in step f1), it is especially preferable to react the compound of formula (7) with 9-borabicyclo[3.3.1]nonane, 9-BBN, in order to obtain borane (7a) as an intermediate:

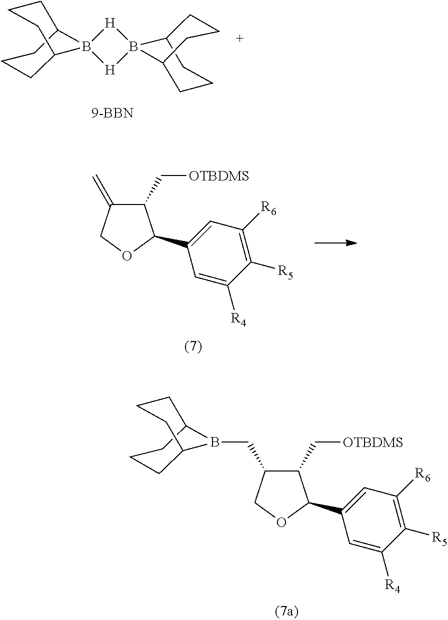

which will be reacted with the suitably substituted phenyl or 2-pyridyl bromide (7b) using the 1,1′-bis(diphenylphosphino)ferrocene-dichloropalladium(II)-dichlormethane complex, Pd(dppf)Cl₂.CH₂Cl₂, as a catalyst to obtain compound (8) in the immediately following step f2). This reaction is generally described, e.g., in Chen et al., Org. Lett. 12(7), 1377-1379 (2010).

Due to the bulky boryl residue and the bulkiness of the protecting group pointing “downwards” (relative to the sheet plane), attacking the double bond can substantially only be done “from above”. Since the hydride hydrogen reacts with the proximal carbon atom, it will be bound above the plane of this planar sp2-hybridized carbon ring atom, thus altering the hybridization of the carbon ring atom from sp2 into the tetrahedral sp3 form. Binding to the distal carbon atom is therefore inevitably occurring “downwards”, thereby forming the desired 3,4-cis configuration. A scheme of this reaction mechanism can be found in FIG. 2, wherein TBDMS was assumed as the protecting group.

Subsequently, the actual Suzuki coupling is performed, i.e. coupling the hydroborylated compound (7a) with the halogenated aromatic (7b), i.e. herein preferably with an appropriately substituted phenyl or 2-pyridyl bromide or iodide, although basically other leaving groups such as triflate or other aromatic compounds such as naphthyl or further heteroaryl groups may also be used analogously as the compound (7b).

The compound (8) thereby obtained is already either corresponding to formula (II) if, in the previous step e), the desired acyl, alkyl, or aryl residue has already been attached to the OH group as a “protecting group”, or cleaving the alcohol protecting group will be conducted in step g) to give the free alcohol (9). Preferably, this is done using tetra-n-butylammonium fluoride, TBAF, since this constitutes a reliable method for removing silyl protecting groups comprising reaction conditions that are compatible with the novel compounds.

In step h), the free OH group of (9) is finally reacted with the desired acyl, alkyl or aryl residue, which again allows for the production of a wide range of novel compounds of formula (II) within a single step.

In appropriately selecting reactants and reaction methods, several consecutive steps of the method according to the invention, such as steps f) and g), or steps g) and h), may be conducted via a one-pot reaction.

Preferably, in step e) or h), Mitsunobu esterification or Steglich esterification using a free acid which serves as the protecting group and/or forms the respective residue R₇ will be conducted to esterify the OH group, wherein, more preferably, Mitsunobu esterification will be conducted in the presence of 1,1′-(azodicarbonyl)dipiperidine, ADD, or diethyl azodicarboxylate, DEAD, and triphenylphosphane, PPh₃, and Steglich esterification will be conducted in the presence of N-(3-Dimethylaminopropyl)-N′-ethylcarbodiimide, EDC, or N,N′-Dicyclohexylcarbodiimide, DCC, and 4-Dimethylaminopyridine, DMAP.

Compared to other established esterification methods, this allows for either activating the alcohol (Mitsunobu) or the carboxylic acid (Steglich) depending on the chemical requirements, for example, if incompatibilities of the reactants with one of the reaction conditions should occur, e.g. during the activation of angelic acid.

### BRIEF DESCRIPTION OF DRAWINGS

In the accompanying drawings:

FIG. 1 depicts the synthetic route for preparing Leoligin and derivatives thereof, which is known from literature;

FIG. 2 shows the hydroboration mechanism of step f) of the method according to the invention; and

FIGS. 3a and 3b show an overview of the synthetic route according to the method of the present invention.

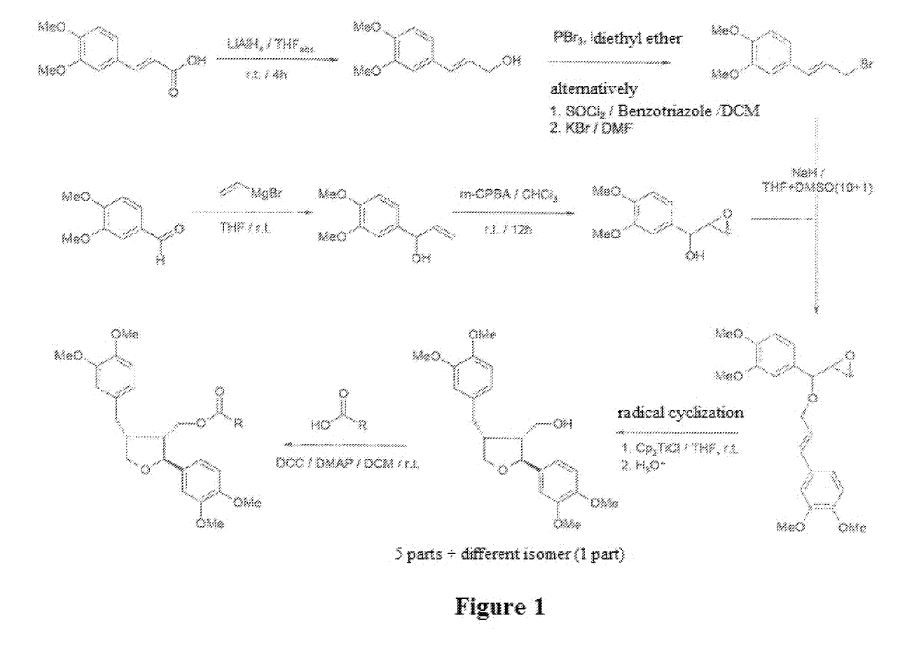

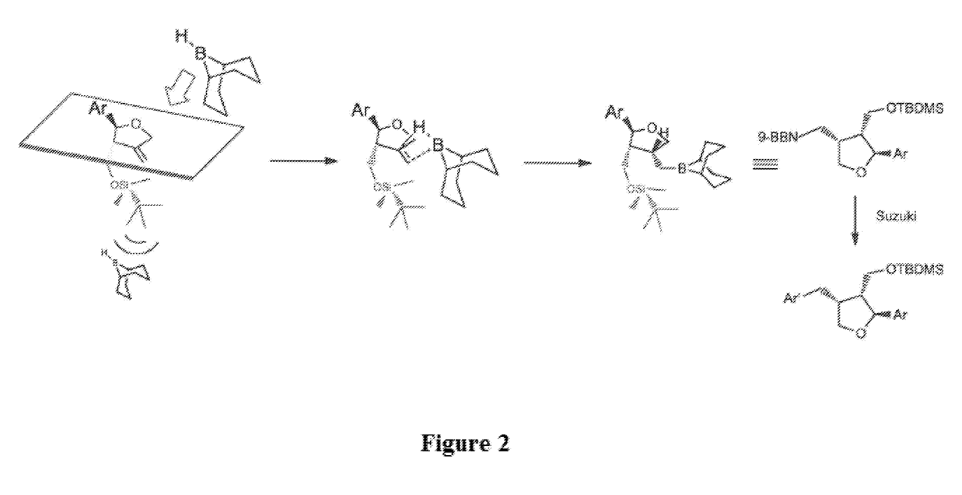

Hereinafter, the present invention will now be described in more detail with reference to non-limiting examples and comparative examples.

### EXAMPLES

### General Remarks

Chemicals were purchased from commercial suppliers and used without further purification unless noted otherwise.

Dry Solvents were obtained by passing pre-dried material through a cartridge containing activated alumina via a solvent dispensing system unless noted otherwise, and stored under dry nitrogen.

Dry and deoxygenated THF was obtained by refluxing the solvent over sodium-benzophenone under argon and subsequent distillation.

Cooling

A Cryostat RKT20 Lauda was used to provide low temperatures for prolonged low-temperature reactions.

### Preparative Chromatography

Flash column chromatography was performed on a Buchi Sepacore MPLC system, using silica gel 60 (40-63 μm) from Merck. Preparative HPLC was performed on a Shimadzu LC-8A device with a SIL-10AP autosampler, SPD-20 detector and FRC-10A fraction collector.

### Specific Rotatation

An Anton Parr MCP500 polarimeter was used to measure specific rotation values.

### High Resolution Mass Spectroscopy (E. Rosenberg, CTA, TU Wien)

Samples were analyzed by LC-IT-TOF-MS using ESI (positive ion mode) or APCI-PI, and the M+H⁺ or M+Na⁺ quasi-molecular ions were used for mass determination. LC: Shimadzu Prominence, consisting of a solvent degassing unit (DGU-20 A3), binary gradient pump (2×LC-20AD), auto-injector (SIL-20A), column oven (CTO-20AC), control module (CBM-20A), and diode array detector (SPD-M20A). MS: Shimadzu IT-TOF-MS with atmospheric pressure chemical ionization and electrospray interface.

### NMR Spectroscopy

¹H- and ¹³C-NMR spectra were recorded from CDCl₃ or DMSO-d₆ solutions on a Bruker AC 200 (200 MHz) or on a Bruker Avance UltraShield 400 (400 MHz) spectrometer. Chemical shifts are reported in ppm.

### ABBREVIATIONS

act. MS activated molecular sieves

ADD 1,1′-(Azodicarbonyl)dipiperidine

9-BBN 9-Borabicyclo[3.3.1]nonane

DEAD diethyl azodicarboxylate

DET diethyl tartrate

DIAD diisopropyl azodicarboxylate

DIPEA diisopropylethylamine

4-DMAP 4-dimethylaminopyridine

DMSO dimethyl sulfoxide

DPPA diphenyl phosphorazidate

dppf 1,1′-bis(diphenylphosphino)ferrocene

EDCI.HCl N-(3-dimethylaminopropyl)-N′-ethylcarbodiimide hydrochloride

LP light petroleum

mCPBA meta-chloroperbenzoic acid

MTBE methyl tert-butyl ether

TBAF tetra-n-butylammonium fluoride

TBDMS tert-butyldimethylsilyl

TBHP tert-butyl hydroperoxide

THF tetrahydrofuran

### General Reaction Scheme

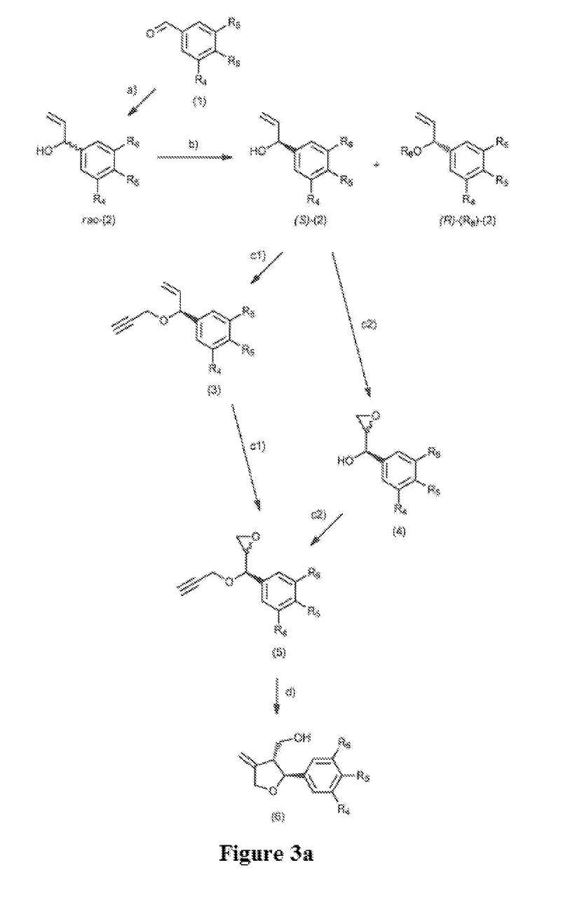

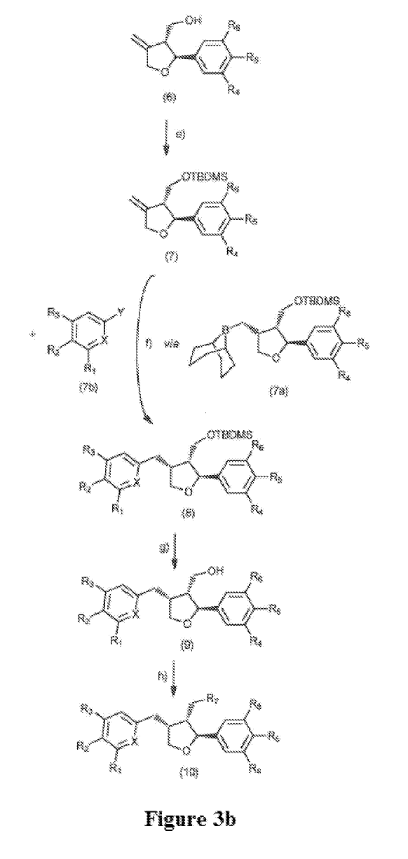

A reaction scheme illustrating the general reaction steps a) through h) described above is shown in FIGS. 3a and 3b.

### Synthesis Examples 1 to 15

In the following, synthesis of intermediates of formulas (2) to (7) are described first. Designation of compounds was done by giving the respective number of formula 1 to 7 (without brackets), each followed by a different lowercase letter “a” to “e”, respectively, to demarcate a specific combination of residues R₄ to R₆ within formula (II). In this, all letters represent one of the following substitution patterns:

“a” R₄=R₅=OCH₃, R₆=H

“b” R₄=R₅=R₆=OCH₃

“c” R₄=R₆=H, R₅=OCH₃

“d” R₄=R₅=R₆=H

“e” R₄=R₆=H, R₅=F

For some compounds, the number of each formula is preceded by a specification regarding stereochemistry, i.e. either “(R)” or “(S)” for each enantiomer, or “rac-” for racemic mixtures.

This is followed by the chemical name of each compound.

### Synthetic Example 1

Exemplified is the synthesis of 6a via intermediates rac-2a, (S)-2a, 3a and 5a, starting from 1a:

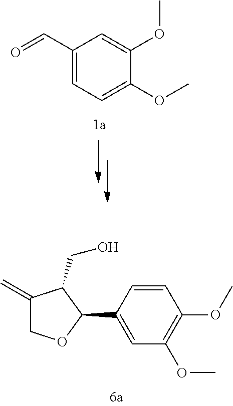

### rac-2a: rac-1-(3,4-dimethoxyphenyl)prop-2-en-1-ol

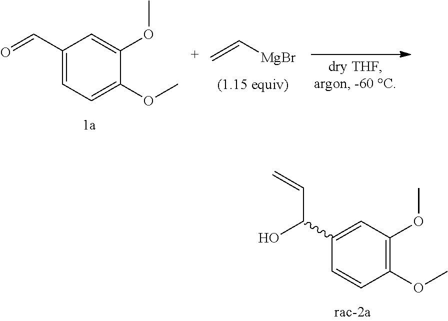

A stirred solution of 3,4-dimethoxybenzaldehyde 1a (73.1 g, 440.0 mmol, 1.00 equiv.) in dry THF (600 mL) under argon was cooled to −60° C., to which was added vinylmagnesium bromide solution (1 M in THF, 506 mL, 506.0 mmol, 1.15 equiv.) via a dropping funnel over a period of 1.8 h while the temperature was kept within ±1° C.

Then the mixture was allowed to warm to −10° C. within 2 h, before a saturated aqueous NH₄Cl solution (100 mL) was added dropwise over 5 min while providing additional cooling to prevent the temperature from rising over +10° C. during the exothermic hydrolysis. To dissolve the magnesium salts, water (450 mL) was added and the product extracted with Et₂O (1×500 mL, 5×250 mL). The combined organic phases were treated with saturated aqueous NaHCO₃ solution (1×150 mL) and saturated brine (1×100 mL), followed by drying with Na₂SO₄. The solution was filtered through a plug of silica (15 g, pre-conditioned with Et₂O) and the solvents were removed in vacuo to afford rac-2a as pale yellow oil (85.4 g, 99%) to be used directly in the next step.

### Synthetic Example 2

### (S)-2a: (S)-1-(3,4-dimethoxyphenyl)prop-2-en-1-ol

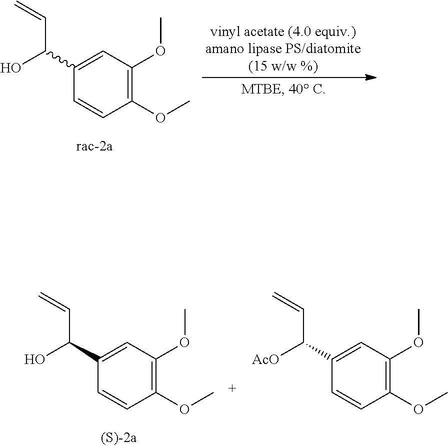 [SMILES: C=CC(O)c1ccc(OC)c(OC)c1 ; C=C[C@H](O)c1ccc(OC)c(OC)c1 ; C=C[C@@H](OC(C)=O)c1ccc(OC)c(OC)c1]

To a mechanically stirred solution of alcohol rac-2a (85.4 g, 439.7 mmol, 1.00 equiv.) and vinyl acetate (151.5 g, 162 mL, 1.76 mol, 4.00 equiv.) in MTBE (2.4 L) at 40° C. was added amano lipase PS (immobilized on diatomite, 12.82 g, 15 w/w %). The resulting suspension was stirred at this temperature for 44.5 h, before the mixture was filtered through celite 545 and the solvent removed in vacuo. Flash column chromatography was performed splitting the crude material in batches as follows:

1.) 10.4 g crude, silica (9 g precolumn, 90 g separation column), 50 mL/min, EtOAc in LP: 15% for 30 min, then 15 to 40% within 80 min.

2.) 15.0 g crude, silica (9 g precolumn, 90 g separation column), 50 mL/min, EtOAc in LP: 15% for 55 min, then 15 to 40% within 40 min.

3.) 20.5 g crude, silica (40 g & 90 g separation columns), 50 mL/min, EtOAc in LP: 15% for 40 min, then 15 to 25% within 5 min, then 25 to 55% within 40 min.

4.) 53.0 g crude, silica (2×90 g separation columns), 50 mL/min, EtOAc in LP: 11% for 35 min, then 15% for 35 min, then 15 to 25% within 5 min, 25 to 65% within 40 min.

This afforded (S)-2a as a pale yellow oil which crystallized to a colorless powder upon standing (34.4 g, 40%).

### Synthetic Example 3

### 3a: (S)-1,2-dimethoxy-4-(1-(prop-2-yn-1-yloxy)allyl)benzene

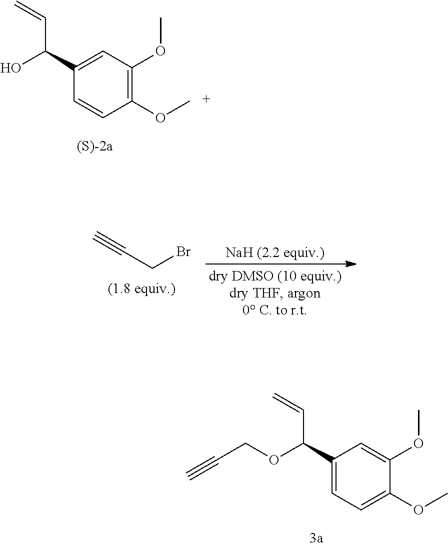

To NaH (approximately 60% dispersion in mineral oil, 15.55 g, 388.7 mmol, 2.20 equiv.) in dry THF (300 mL) under argon was added dry DMSO (125 mL, 1.76 mol, 10.00 equiv.) and the resulting stirred suspension was cooled using an ice bath. Then was added a solution of (S)-2a (34.32 g, 176.7 mmol, 1.00 equiv.) in dry THF (300 mL) via a dropping funnel over a period of 30 min. After that, stirring was continued at that temperature for 15 min, before a solution of propargyl bromide (80% in toluene, 35.4 mL, 318.1 mmol, 1.80 equiv.) was added over a period of 30 min. To loosen up the so formed slurry, more dry THF (300 mL) was added, the ice bath was then removed and the reaction stirred for 13 h. The mixture, still under argon, was then cooled in an ice bath again and hydrolyzed by dropwise addition of aqueous HCl solution (1 M, 100 mL) over 10 min. Most of the THF was then removed in vacuo (40° C.), followed by the addition of water (400 mL) and the crude extracted with Et₂O (4×400 mL), the combined organic phases were treated with saturated brine (1×400 mL), dried with Na₂SO₄, filtered and the solvent removed in vacuo to afford crude 3a (47.23 g) to be used directly in the next step.

### Synthetic Example 4

### 5a: 2-((R)-(3,4-dimethoxyphenyl)(prop-2-yn-1-yloxy)methyl)oxirane

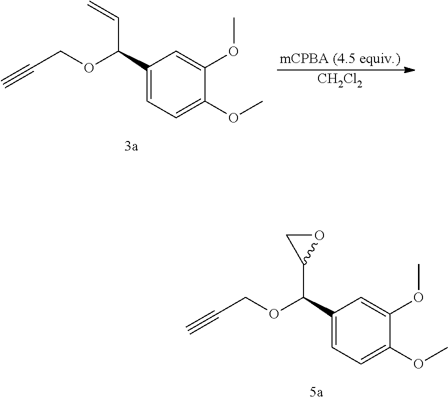

A stirred solution of crude 3a (47.22 g) in CH₂Cl₂ (500 mL) was cooled in an ice bath and mCPBA (approximately 77%, 178.2 g, 795.2 mmol, 4.5 equiv.) was added in small portions over 30 min. The reaction was allowed to warm to room temperature while stirring was continued for 17 h. Then was added sufficient aqueous Na₂SO₃ solution to destroy residual peroxy acid, which required cooling using an ice bath. This was followed by an aqueous solution of K₃PO₄ (185 g) in water (750 mL) to bring the pH to 8. The crude product was extracted with Et₂O (1×750 mL, 5×250 mL), the combined organic phases were treated with saturated brine (250 mL), dried with Na₂SO₄ and the solvent removed from the filtrate in vacuo to afford crude 5a (50.09 g) to be used directly in the next step.

### Synthetic Example 5

### 6a: ((2S,3R)-2-(3,4-dimethoxyphenyl)-4-methylenetetrahydrofuran-3-yl)methanol

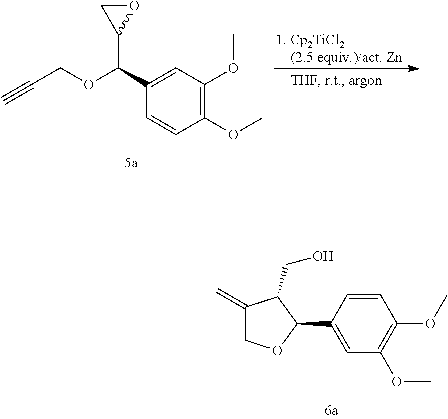

To dry and deoxygenated THF (2.5 L) under argon was added bis(cyclo-pentadienyl)titanium(IV) dichloride (108.6 g, 436.1 mmol, 2.5 equiv.) and activated zinc dust (79.8 g, 1.22 mol, 7.0 equiv.), and the resulting suspension stirred vigorously at room temperature for 1 h. Then the residual zinc was allowed to settle for 5 min, after which the green solution was transferred via a canula to a fast stirred solution of crude epoxide 5a (49.42 g) in dry and deoxygenated THF (1.2 L) at room temperature over a period of 3 h. After that, stirring was continued for another 1.25 h, then was carefully added dilute H₂SO₄ (10%, 1 L) and most of the THF removed in vacuo (down to 150 mbar at 40° C.). The crude product was then extracted with Et₂O, the combined organic layers treated with saturated aqueous NaHCO₃ solution (500 mL), saturated brine (250 mL), dried with Na₂SO₄, filtered and the solvent removed from the filtrate in vacuo.

Flash column chromatography was performed on the entire batch in sequence as follows:

1.) silica (2×90 g separation columns), 40 mL/min, EtOAc in LP: 25 to 50% within 45 min, then 50 to 100% within 180 min.

2.) silica (1×90 g separation column), 40 mL/min, EtOAc in LP: 15 to 50% within 45 min, then 50 to 100% within 180 min.

This afforded 6a as a brown oil (8.62 g, 20% over 3 steps).

¹H-NMR (200 MHz, CDCl₃): δ 1.63 (bs, 1H), 2.71-2.86 (m, 1H), 3.63-4.00 (m, 8H), 4.42 (dq, J=13.4, 2.2 Hz, 1H), 4.62 (d, J=13.4 Hz, 1H), 4.79 (d, J=7.5 Hz, 1H), 5.07 (q, J=2.3 Hz, 1H), 5.12 (q, J=2.2 Hz, 1H), 6.79-6.99 (m, 3H).

¹³C-NMR (50 MHz, CDCl₃): δ 54.0, 56.0, 56.0, 62.0, 71.5, 83.5, 105.1, 109.4, 111.0, 119.0, 133.6, 148.9, 149.0, 149.3.

### Synthetic Examples 6 to 8

Compounds 6c, 6d and 6e were obtained in analogy to 6a, via the corresponding intermediates rac-2, (S)-2, 3, and 5, starting from 1c, rac-2d and 1e, respectively:

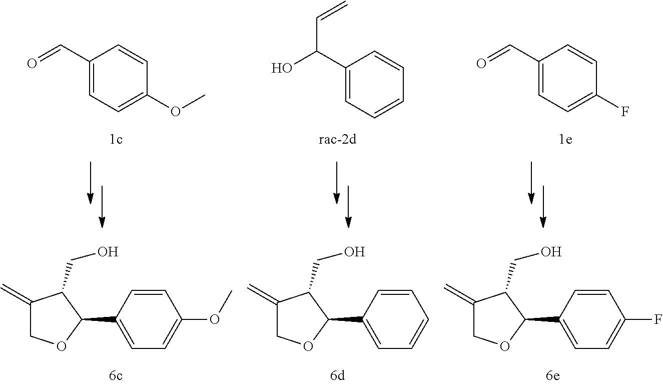

### Syntheses 9 and 10

Compound 6b was obtained via intermediates rac-2b, (S)-2b, 4b and 5b, starting from 1b. Synthesis of rac-2b, (S)-2b and 6b was analogous to that of rac-2a, (S)-2a and 6a, respectively. The synthesis of 4b and 5b is given below.

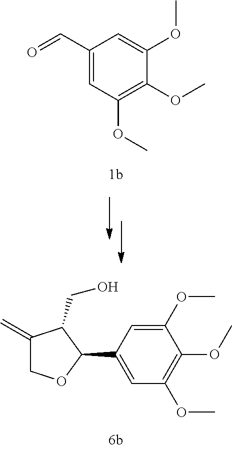

### Synthetic Example 9

### 4b: (1R)-oxiran-2-yl(3,4,5-trimethoxyphenyl)methanol

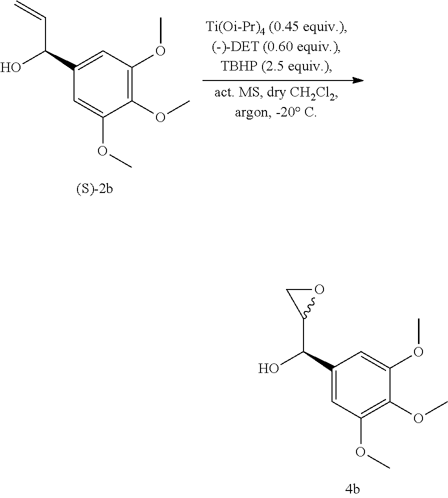 [SMILES: C=C[C@H](O)c1cc(OC)c(OC)c(OC)c1 ; COc1cc([C@@H](O)C2CO2)cc(OC)c1OC]

Dry CH₂Cl₂ (150 mL), (−)-DET (2.789 g, 12.53 mmol, 0.6 equiv.) and (S)-2b (5.085 g, 22.67 mmol, 1.0 equiv.) were (additionally) dried over activated molecular sieves overnight under argon atmosphere. (−)-DET was dissolved in dried CH₂Cl₂ (1.0 mL) and cooled to −20° C. via a cryostat. Ti(Oi-Pr)₄ (3.00 mL, 10.145 mmol, 0.45 equiv.) in dry CH₂Cl₂ (70 mL, 0.14 M) was added and the reaction mixture was stirred for 15 min. Then TBHP (5.5 M in decane, 10.3 mL, 56.68 mmol, 2.5 equiv) was added slowly. After 30 min a solution of (S)-2b in CH₂Cl₂ was added and the resulting mixture was stirred for 70 h at −20° C. Following that, a solution of Na₂SO₃ 20 g in 100 mL water) was added as well as 1000 mL CH₂Cl₂ and 500 mL water. The aqueous layer was extracted with CH₂Cl₂ (4×500 mL) and the combined organic layers were dried over Na₂SO₄ and filtered. The solvent was removed in vacuo and flash column chromatography was performed (crude product adsorbed onto BULK Isolute® HM-N, 90 g silica, flow rate 50 mL/min, isocratically 8% EtOAc in LP for 15 min, then gradient 8 to 50% within 25 min, then 50 to 100% within 10 min, then isocratically 100% for 10 min). This afforded 4b as an orange oil (4.431 mg, 81%).

### Synthetic Example 10

### 5b: 2-((R)-(prop-2-yn-1-yloxy)(3,4,5-trimethoxyphenyl)methyl)oxirane

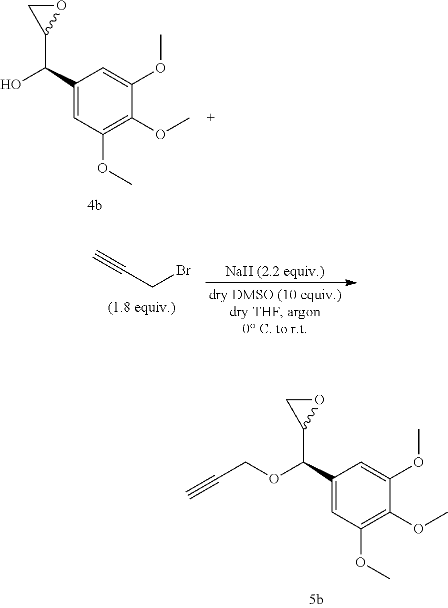

To NaH mineral oil dispersion (approximately 60%, 1.62 g, 40.57 mmol, 2.2 equiv.) under argon was added dry THF (such as to achieve a 1.0 M suspension with respect to NaH) and cooled to 0° C. in an ice bath, followed by the addition of dry DMSO (13.1 mL, 184 mmol, 10 equiv.) via syringe. This was followed by the dropwise addition of 5b (4.431 g, 18.44 mmol, 1.0 equiv.) as a solution in dry THF (0.4 M), subsequent stirring of the reaction for 15 min at 0° C., and finally by propargyl bromide (80% solution in toluene, 3.7 mL, 33.2 mmol, 1.8 equiv.) as a solution in dry THF, both via syringe. The ice bath was removed and the mixture stirred for 48 h at room temperature. Following that, the reaction was cooled to 0° C. again and quenched by the dropwise addition of aqueous HCl (1 M, 1.0 equiv.). Most of the THF was evaporated and water was then added. The mixture was extracted with Et₂O (4×), the combined organic phases treated with saturated brine, dried with Na₂SO₄, filtered and the solvents evaporated. Flash column chromatography was then performed (90 g silica, flow rate 50 mL/min, gradient, isocratically 3% EtOAc in LP for 10 min, then 3 to 30% within 20 min, then 30 to 100% within 20 min, then isocratically 100% for 10 min). This afforded 5b as a slightly yellow oil (4.211 g, 82%).

### Synthetic Example 11

### Examplified is the Synthesis of 7a from 6a

### 7a: tert-butyl(((2S,3R)-2-(3,4-dimethoxyphenyl)-4-methylenetetrahydrofuran-3-yl)-methoxy)dimethylsilane

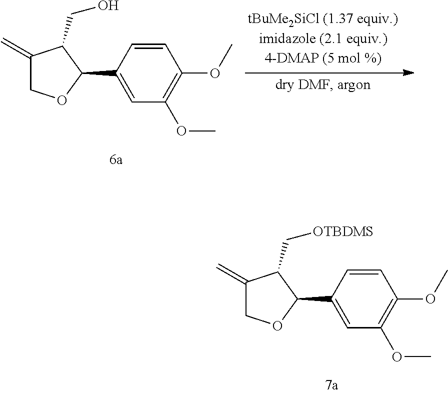

To a stirred solution of 6a (1.723 g, 6.885 mmol, 1.00 equiv.), imidazole (984 mg, 14.459 mmol, 2.10 equiv.) and 4-dimethylaminopyridine (42 mg, 0.344 mmol, 0.05 equiv.) in dry DMF (40 mL) under argon was added dropwise tert-butylchloro-dimethylsilane solution (3 M in THF, 3.14 mL, 9.42 mmol, 1.37 equiv.), and the mixture stirred at room temperature for 12.5 h. Then was added Et₂O (100 mL), followed by saturated aqueous NH₄Cl solution (40 mL). The layers were separated, the aqueous phase extracted with Et₂O (3×50 mL), the combined organic phases treated with a saturated aqueous solution of NaHCO₃ (25 mL), saturated brine (25 mL), dried with Na₂SO₄, filtered and the solvents removed in vacuo to afford crude 7a (2.63 g) to be used directly for hydroboration-Suzuki coupling in the next step.

### Synthetic Examples 12 to 15

Compounds 7b, 7c, 7d and 7e were obtained in analogy to 7a from 6b, 6c, 6d and 6e, respectively, and used directly for hydroboration-Suzuki coupling in the next step.

### Examples 1 to 6 and Comparative Examples 1 to 8

Compounds synthesized in the subsequent sections are those of biological interest (i.e. corresponding to formula (II)), and most of them were (already) submitted to biological activity screening, whereupon they were alotted a 4-digit “bio ID” (Leoligin, for example, has the ID “2418”), selected according to the compound's chemical name. If no “bio ID” is stated, the compound in question either has not been tested yet, or did not exhibit the desirable selectivity for inhibiting cell proliferation and therefore constitutes a Comparative Example. Furthermore, each compound showing an activity ratio of less than 1.5 in activity assays (which will be described in more detail below) are designated as Comparative Examples, although, as was mentioned above, future tests could possibly still demonstrate their usefulness for the purpose of the invention.

Attaching the aryl residue carrying the substitutes R₁ to R₃ was consistently done via Suzuki coupling after initial hydroboration. The consecutive cleavage of the OH protecting group was usually conducted as a one-pot reaction, i.e. without isolating the protected intermediate of formula (8).

Compound designations were formed by stating the number of the formula, 9, followed by another, basically consecutively increasing number. Missing compound numbers in this row indicate that the respective compounds have not exhibited the desired selectivity for inhibiting cell proliferation (yet) (or have not been tested for it yet), and have not been produced neither as starting materials for compounds with desired activity, nor by means of reference synthesis.

The experimental description is usually divided into “preparation” and “work-up and purification”, and in many cases is referred back to an example further up in the list if preparation and/or work-up and purification was identical or analogous.

Usually, ¹H-NMR, HR-MS and specific rotation or, alternatively, ¹H-NMR, ¹³C-NMR and specific rotation are given for compounds for which biological results are available. When the preparation procedure for such a compound is given explicitly in a particular example (i.e. not referenced to an identical or analogous procedure), ¹H-NMR, ¹³C-NMR, HR-MS and specific rotation are given.

Within this first group of examples and comparative examples, only intermediate 7a, with R₄=R₅=OCH₃ and R₆=H, was derivatized.

### Comparative Example 1

### 9-1: ((2S,3R,4R)-4-(3,4-dimethoxybenzyl)-2-(3,4-dimethoxyphenyl)tetrahydrofuran-3-yl)methanol (dimethyl lariciresinol)

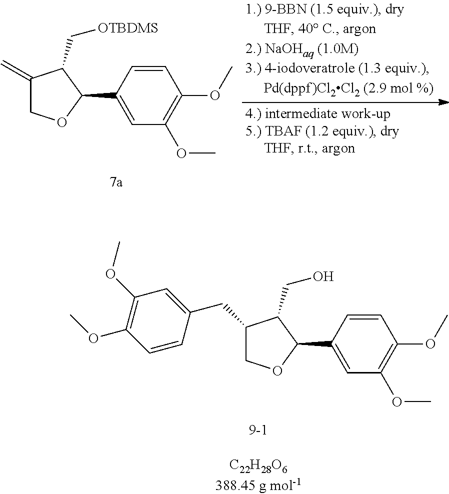

Preparation: a reaction vessel was charged with a stir bar and crude starting material 7a (2.51 g, 6.88 mmol, 1.0 equiv.), and then evacuated and back-filled with argon using standard Schlenk technique (3×). Then was added a solution of 9-BBN (0.5 M in THF, 20.7 mL, 10.3 mmol, 1.5 equiv.) via syringe, the reaction stirred for 16.5 h at 40° C. and then allowed to cool to room temperature. Following this, degassed aqueous solution of NaOH (2M, 20 mL) was added cautiously and stirring continued for another 15 min. Then was added 4-iodoveratrole (2.36 g, 8.95 mmol, 1.30 equiv.) and Pd(dppf)Cl₂.CH₂Cl₂ (161 mg, 0.198 mmol, 2.9 mol %), and the resulting biphasic mixture stirred vigorously at room temperature for 25 h. Then was added Et₂O (200 mL) and saturated brine (50 mL), the layers were separated, the aqueous phase extracted with Et₂O (4×50 mL), the combined organic phases dried with Na₂SO₄ and filtered. Using a new reaction vessel, the solvent was evaporated, to the residue was added a stir bar and then evacuated and back-filled with argon. For deprotection, a solution of TBAF (1.0 M in THF, 8.25 mL, 8.25 mmol, 1.2 equiv.) was added via syringe and the mixture finally stirred for 18 h at room temperature.

Work-up and purification: Et₂O (200 mL) and saturated brine (50 mL) were added, the layers were separated and the aqueous phase extracted with Et₂O (4×50 mL) and EtOAc (2×50 mL). The combined organic phases were dried with Na₂SO₄, filtered and the solvents evaporated. The target compound was purified by flash column chromatography in two subsequent runs (first run: 90 g silica with 9 g pre-column, flow rate 40 mL/min, isocratically at 30% EtOAc in LP for 3 min, then gradient 30 to 100% within 60 min; second run: 90 g silica, flow rate 40 mL/min, gradient 45 to 85% EtOAc in LP within 60 min) to give a slightly colored oil (1.04 g, 39%).

¹H-NMR (200 MHz, CDCl₃): δ 1.52 (bs, 1H), 2.42 (quint, J=6.9 Hz, 1H), 2.56 (dd, J=12.7, 10.4 Hz, 1H), 2.66-2.85 (m, 1H), 2.94 (dd, J=12.8, 4.7 Hz, 1H), 3.73-3.98 (m, 2H), 3.76 (dd, J=8.5, 5.9 Hz, 1H), 3.87 (s, 9H), 3.88 (s, 3H), 4.07 (dd, J=8.5, 6.4 Hz, 1H), 4.81 (d, J=6.5, 1 H), 6.70-6.81 (m, 3H), 6.81-6.91 (m, 3H).

¹³C-NMR (50 MHz, CDCl₃): δ 33.4, 42.5, 52.7, 56.0, 56.1, 61.1, 73.1, 82.9, 109.0, 111.1, 111.4, 112.0, 118.2, 120.6, 133.1, 135.5, 147.6, 148.5, 149.1, 149.2.

### Comparative Example 2

### 9-2: ((2S,3R,4R)-4-(3,4-dimethoxybenzyl)-2-(3,4,5-trimethoxyphenyl)tetrahydrofuran-3-yl)methanol

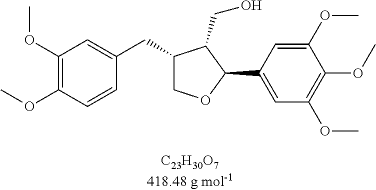

Preparation analogous to that of 9-1, using crude starting material 7b (1.48 g, 3.62 mmol, 1.0 equiv.) and stirring for 22.5 h, 24 h and 20 h in the hydroboration, coupling and deprotection steps, respectively.

Work-up and purification: Et₂O (100 mL) and saturated brine (25 mL) was added and the aqueous phase extracted with Et₂O (4×30 mL) and EtOAc (2×35 mL). The combined organic phases were dried with Na₂SO₄, filtered and the solvents evaporated. The target compound was purified by flash column chromatography (90 g silica, flow rate 40 mL/min, isocratically at 30% EtOAc in LP for 5 min, then gradient 30 to 100% within 45 min) to give a yellow oil (631 mg, 42%).

[α]_{D}²⁰: +7.1 (c 2.14, MeOH)

HR-MS (ESI) calc'd for M+Na⁺: 441.1884. found: 441.1903.

¹H-NMR (200 MHz, CDCl₃): δ 1.68 (bs, 1H), 2.32-2.82 (m, 3H), 2.92 (dd, J=12.8, 4.6 Hz, 1H), 3.83 (s, 3H), 3.85 (s, 15H), 4.07 (dd, J=8.4, 6.5 Hz, 1H), 4.84 (d, J=6.1 Hz, 1H), 6.56 (s, 2H), 6.67-6.83 (m, 3H).

¹³C-NMR (50 MHz, CDCl₃): δ 33.1, 42.2, 52.4, 55.8, 56.1, 60.8, 60.9, 73.0, 82.9, 102.5, 111.2, 111.8, 120.4, 132.9, 137.1, 138.7, 147.4, 148.9, 153.2.

### Comparative Example 3

### 9-3: ((2S,3R,4R)-4-benzyl-2-(3,4-dimethoxyphenyl)tetrahydrofuran-3-yl)methanol

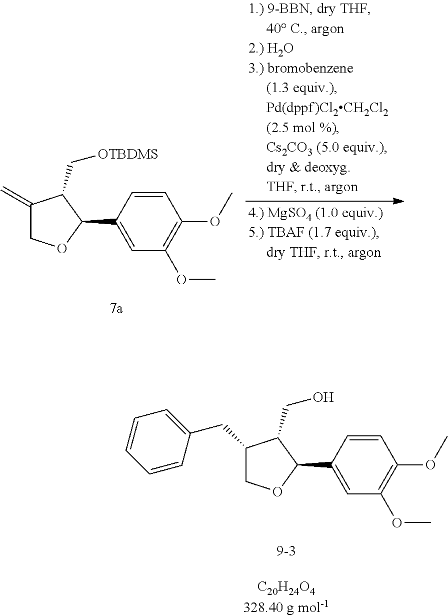

Preparation: a reaction vessel was charged with a stir bar and crude starting material 7a (715.9 mg, 1.964 mmol), and then evacuated and back-filled with argon using standard Schlenk technique (3×). Then was added a solution of 9-BBN (0.5 M in THF, 5.89 mL, 2.95 mmol) via syringe, the reaction stirred for 21 h at 40° C. and then allowed to cool to room temperature. Water (35 μL, 2.0 mmol) was subsequently added and stirring continued for 2 h to decompose excess 9-BBN.

This mixture was then purged by bubbling argon into the solution through a needle, and then was added dry and deoxygenated THF to produce a total volume of 10.0 mL, i.e. a 0.196 M solution of borylated intermediate, thus allowing the use of aliquots for subsequent coupling.

An aliquot (0.97 mL, 0.19 mmol, 1.0 equiv.) of this solution was then transferred via syringe to a separate vessel which had been charged with a stir bar, bromobenzene (38.8 mg, 0.247 mmol, 1.3 equiv.), Pd(dppf)Cl₂.CH₂Cl₂ (3.9 mg, 4.8 μmol, 2.5 mol %) and Cs₂CO₃ (310 mg, 0.950 mmol, 5.0 equiv.) under argon and then stirred for 36 h at room temperature. Following this, MgSO₄ (23 mg, 0.19 mmol, 1.0 equiv.) was added and stirring continued for 1.5 h to remove residual water. For deprotection, a solution of TBAF (1.0 M in THF, 0.32 mL, 0.32 mmol, 1.7 equiv.) was added via syringe and the mixture finally stirred for 32 h at room temperature.

Work-up and purification: the heterogeneous reaction content was filtered and rinsed with CH₂Cl₂ (20 mL), the solvents evaporated and the target compound purified by flash column chromatography (18 g silica, flow rate 20 mL/min, gradient 20 to 70% EtOAc in LP within 30 min) to give a light brown oil (39.3 mg, 63%).

[α]_{D}²³: +18.3 (c 3.80, MeOH)

HR-MS (ESI) calc'd for M+Na⁺: 351.1567. found: 351.1577.

¹H-NMR (200 MHz, CDCl₃): δ 1.70 (bs, 1H), 2.40 (quint, J=6.8 Hz, 1H), 2.61 (dd, J=12.5, 10.2 Hz, 1H), 2.67-2.87 (m, 1H), 2.96 (dd, J=12.6, 4.6 Hz, 1H), 3.69-3.98 (m, 3H), 3.85 (s, 3H), 3.87 (s, 3H), 4.05 (dd, J=8.5, 6.4 Hz, 1H), 4.83 (d, J=6.3 Hz, 1H), 6.77-6.94 (m, 3H), 7.14-7.35 (m, 5H).

¹³C-NMR (50 MHz, CDCl₃): 33.7, 42.2, 52.5, 56.0, 56.0, 61.0, 73.0, 82.9, 109.0, 111.1, 118.1, 126.3, 128.7, 128.7, 135.6, 140.5, 148.4, 149.1.

### Example 1

### 9-5: ((2S,3R,4R)-4-(4-(tert-butyl)benzyl)-2-(3, 4-dimethoxyphenyl)tetrahydrofuran-3-yl)methanol (2749)

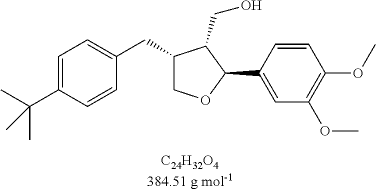 [SMILES: COc1ccc([C@H]2OC[C@H](Cc3ccc(C(C)(C)C)cc3)[C@@H]2CO)cc1OC]

Preparation identical to that of 9-3 except for using 1-bromo-4-(tert-butyl)benzene (52.6 mg, 0.247 mmol, 1.3 equiv.) as aryl halide coupling partner.

Work-up and purification: the heterogeneous reaction content was filtered and rinsed with CH₂Cl₂ (20 mL), the solvents evaporated and the target compound purified by flash column chromatography (18 g silica, flow rate 20 mL/min, gradient 20 to 70% EtOAc in LP within 30 min) to give a light brown oil (49.1 mg, 67%).

[α]_{D}²⁵: +12.0 (c 4.91, MeOH)

HR-MS (ESI) calc'd for M+Na⁺: 407.2193. found: 407.2183.

¹H-NMR (200 MHz, CDCl₃): δ 1.31 (s, 9H), 1.68 (bs, 1H), 2.40 (quint, J=6.7 Hz, 1H), 2.59 (dd, J=12.5, 9.8 Hz, 1H), 2.67-2.86 (m, 1H), 2.92 (dd, J=12.6, 4.8 Hz, 1H), 3.69-3.98 (m, 3H), 3.86 (s, 3H), 3.87 (s, 3H), 4.08 (dd, J=8.5, 6.4 Hz, 1H), 4.83 (d, J=6.2 Hz, 1H), 6.78-6.94 (m, 3H), 7.12 (d, J=8.2 Hz, 2H), 7.31 (d, J=8.2 Hz, 2H).

### Comparative Example 4

### 9-6: ethyl 4-(((3R,4R,5S)-5-(3,4-dimethoxyphenyl)-4-(hydroxymethyl)tetrahydrofuran-3-yl)methyl)benzoate

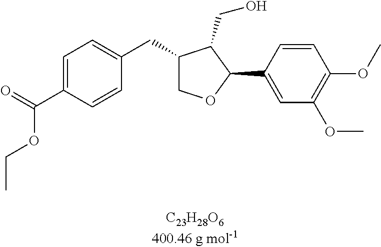 [SMILES: CCOC(=O)c1ccc(C[C@H]2CO[C@H](c3ccc(OC)c(OC)c3)[C@H]2CO)cc1]

Preparation identical to that of 9-3 except for using ethyl 4-bromobenzoate (56.6 mg, 0.247 mmol, 1.3 equiv.) as aryl halide coupling partner.

Work-up and purification: the heterogeneous reaction content was filtered and rinsed with CH₂Cl₂ (20 mL), the solvents evaporated and the target compound purified by flash column chromatography (18 g silica, flow rate 20 mL/min, gradient 30 to 52% EtOAc in LP within 17 min, then to 74% within 9 min, then isocratically at 74%) to give a light brown oil (47.3 mg, 62%).

[α]_{D}²³: +11.8 (c 4.64, MeOH)

HR-MS (ESI) calc'd for M+Na⁺: 423.1778. found: 423.1795.

¹H-NMR (200 MHz, CDCl₃): δ 1.38 (t, J=7.1 Hz, 3H), 1.86 (t, J=4.6 Hz, 1H), 2.42 (quint, J=6.7 Hz, 1H), 2.59-2.88 (m, 2H), 3.04 (dd, J=11.9 Hz, 3.3 Hz, 1H), 3.71 (dd, J=8.6 Hz, 6.0 Hz, 1H), 3.74-3.99 (m, 2H), 3.86 (s, 3H), 3.87 (s, 3H), 4.03 (dd, J=8.6, 6.2 Hz, 1H), 4.36 (q, J=7.1 Hz, 2H), 4.82 (d, J=6.4 Hz, 1H), 6.78-6.94 (m, 3H), 7.27 (d, J=8.2 Hz, 2H), 7.97 (d, J=8.2 Hz, 2H).

### Example 2

### 9-9: ((2S,3R,4R)-2-(3,4-dimethoxyphenyl)-4-(4-methylbenzyl)tetrahydrofuran-3-yl)-methanol (2754)

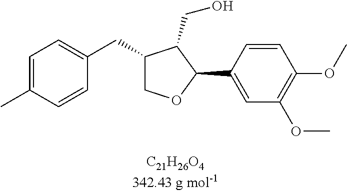

Preparation identical to that of 9-3 except for using 4-bromotoluene (42.2 mg, 0.247 mmol, 1.3 equiv.) as aryl halide coupling partner.

Work-up and purification: the heterogeneous reaction content was filtered and rinsed with CH₂Cl₂ (20 mL), the solvents evaporated and the target compound purified by flash column chromatography (18 g silica, flow rate 20 mL/min, gradient 20 to 70% EtOAc in LP within 30 min) to give a light brown oil (43.2 mg, 66%).

[α]_{D}²³: +15.0 (c 4.34, MeOH)

HR-MS (ESI) calc'd for M+Na⁺: 365.1723. found: 365.1727.

¹H-NMR (200 MHz, CDCl₃): δ 1.72 (t, J=5.0 Hz, 1H), 2.31 (s, 3H), 2.39 (quint, J=6.7 Hz, 1H), 2.57 (dd, J=12.6, 10.0 Hz, 1H), 2.65-2.85 (m, 1H), 2.91 (dd, J=12.6, 4.8 Hz, 1H), 3.66-3.98 (m, 3H), 3.85 (s, 3H), 3.87 (s, 3H), 4.05 (dd, J=8.5, 6.4 Hz, 1H), 4.82 (d, J=6.3 Hz, 1H), 6.77-6.90 (m, 3H), 7.09 (s, 4H).

### Comparative Example 5

### 9-12: ((2S,3R,4R)-2-(3,4-dimethoxyphenyl)-4-(3-fluorobenzyl)tetrahydrofuran-3-yl)-methanol

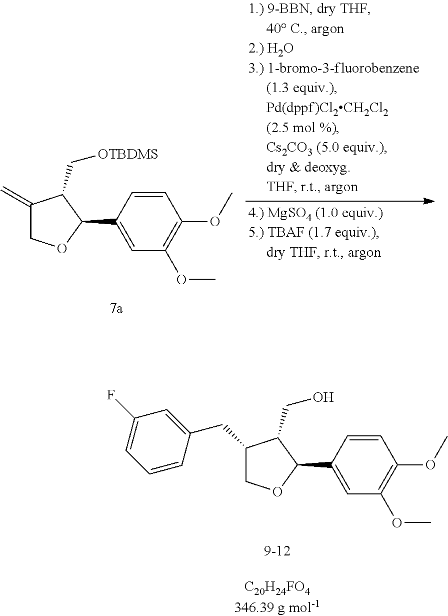

Preparation: a reaction vessel was charged with a stir bar and crude starting material 7a (843.2 mg, 2.313 mmol), and then evacuated and back-filled with argon using standard Schlenk technique (3×). Then was added a solution of 9-BBN (0.5 M in THF, 6.94 mL, 3.47 mmol) via syringe, the reaction stirred for 35 h at 40° C. and then allowed to cool to room temperature. Water (42 μL, 2.3 mmol) was subsequently added and stirring continued for 2 h to decompose excess 9-BBN.

This mixture was then purged by bubbling argon into the solution through a needle, and then was added dry and deoxygenated THF to produce a total volume of 13.0 mL, i.e. a 0.178 M solution of borylated intermediate, thus allowing the use of aliquots for subsequent coupling.

An aliquot (1.07 mL, 0.19 mmol, 1.0 equiv.) of this solution was then transferred via syringe to a separate vessel which had been charged with a stir bar, 1-bromo-3-fluorobenzene (43.2 mg, 0.247 mmol, 1.3 equiv.), Pd(dppf)Cl₂.CH₂Cl₂ (3.9 mg, 4.8 μmol, 2.5 mol %) and Cs₂CO₃ (310 mg, 0.950 mmol, 5.0 equiv.) under argon and then stirred for 50 h at room temperature. Following this, MgSO₄ (23 mg, 0.19 mmol, 1.0 equiv.) was added and stirring continued for 1.5 h to remove residual water. For deprotection, a solution of TBAF (1.0 M in THF, 0.32 mL, 0.32 mmol, 1.7 equiv.) was added via syringe and the mixture finally stirred for 24 h at room temperature.

Work-up and purification: the heterogeneous reaction content was filtered and rinsed with CH₂Cl₂ (20 mL), the solvents evaporated and the target compound purified by flash column chromatography (18 g silica, flow rate 20 mL/min, gradient 15 to 80% EtOAc in LP within 40 min) to give a colorless oil (50.9 mg, 77%).

[α]_{D}²⁵: +21.0 (c 2.22, MeOH)

HR-MS (ESI) calc'd for M+Na⁺: 369.1473. found: 369.1476.

¹H-NMR (200 MHz, CDCl₃): δ 1.78 (bs, 1H, OH), 2.40 (quint, J=6.7 Hz, 1H), 2.61 (dd, J=12.4, 10.6 Hz, 1H), 2.64-2.85 (m, 1H), 2.97 (dd, J=12.5, 4.1 Hz, 1H), 3.71 (dd, J=8.5, 6.2 Hz, 1H), 3.76-3.90 (m, 2H), 3.86 (s, 3H), 3.87 (s, 3H), 4.05 (dd, J=8.5, 6.3 Hz, 1H), 4.81 (d, J=6.4 Hz, 1H), 6.78-7.00 (m, 6H), 7.31-7.18 (m, 1H).

¹³C-NMR (50 MHz, CDCl₃): δ 33.4 (d, J=1.4 Hz), 42.1, 52.4, 56.0, 56.0, 60.9, 72.8, 82.8, 108.9, 111.0, 113.2 (d, J=21.0 Hz), 115.6 (d, J=21.0 Hz), 118.1, 124.4 (d, J=2.8 Hz), 130.1 (d, J=8.4 Hz), 135.4, 143.2 (d, J=7.1 Hz), 148.5, 149.1, 163.0 (d, J=245.8 Hz).

### Comparative Example 6

### 9-13: ((2S,3R,4R)-2-(3,4-dimethoxyphenyl)-4-(pyridin-2-ylmethyl)tetrahydrofuran-3-yl)methanol

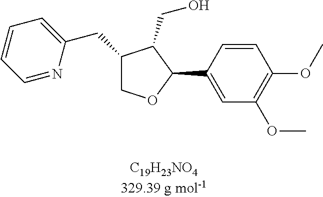 [SMILES: COc1ccc([C@H]2OC[C@H](Cc3ccccn3)[C@@H]2CO)cc1OC]

Preparation identical to that of 9-12 except for using 2-bromopyridine (39.0 mg, 0.247 mmol, 1.3 equiv.) as aryl halide coupling partner, and repeating Steps 4 and 5 as deprotection was found to be incomplete.

Following this, Et₂O (5 mL) was added to the reaction content and the mixture extracted with aqueous HCl (1 M, 2×5 mL). To the combined aqueous layers was then added CH₂Cl₂ (10 mL), followed by the careful addition of solid Na₂CO₃ (1.5 g). The aqueous phase was extracted once more with CH₂Cl₂ (10 mL), the pooled extracts dried with Na₂SO₄ and the solvents evaporated. The target compound was purified first by flash column chromatography (18 g silica, flow rate 20 mL/min, 100% EtOAc) and then preparative HPLC (Phenomenex® Luna 10 u C18(2) 100 A, 250×21.20 mm, flow rate 20.0 mL/min, isocratically at 38% MeOH in water) to give a colorless oil (36.7 mg, 59%).

[α]_{D}²⁵: +22.1 (c 1.91, MeOH)

HR-MS (ESI) calc'd for M+H+: 330.1700. found: 330.1698.

¹H-NMR (200 MHz, CDCl₃): δ 2.41-2.57 (m, 1H), 2.72-2.89 (m, 2H), 3.24 (dd, J=15.5, 9.9 Hz, 1H), 3.69 (dd, J=11.6, 4.1 Hz, 1H), 3.79 (dd, J=8.4, 6.5 Hz, 1H), 3.86 (s, 3H), 3.87 (s, 3H), 3.91-4.01 (m, 1H), 4.25 (dd, J=8.5, 6.3 Hz, 1H), 4.66 (d, J=6.4 Hz, 1H), 6.77-6.90 (m, 3H), 7.11-7.24 (m, 2H), 7.64 (td, J=7.7, 1.8 Hz, 1H), 8.47 (d, J=4.9 Hz, 1H).

### Example 3

### 9-14: ((2S,3R,4R)-2-(3,4-dimethoxyphenyl)-4-(naphthalen-1-ylmethyl)tetrahydro-furan-3-yl)methanol (2821)

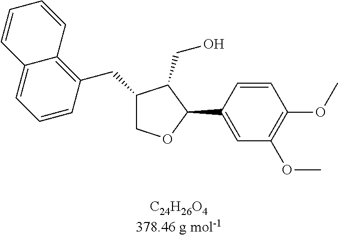 [SMILES: COc1ccc([C@H]2OC[C@H](Cc3cccc4ccccc34)[C@@H]2CO)cc1OC]

Preparation identical to that of 9-12 except for using 1-bromonaphthalene (51.1 mg, 0.247 mmol, 1.3 equiv.) as aryl halide coupling partner.

Work-up and purification: the heterogeneous reaction content was filtered and rinsed with CH₂Cl₂ (20 mL), the solvents evaporated and the target compound purified by flash column chromatography (18 g silica, flow rate 20 mL/min, gradient 15 to 80% EtOAc in LP within 45 min) and then recrystallized from iPrOH (11 mL). The hot solution was decanted from insoluble material, allowed to cool and crystallization then completed by storing at −25° C. Removal of the mother liquor and rinsing with pentane (approximately 3 mL) gave colorless crystals (43.7 mg, 61%).

[α]_{D}²⁰: +39.5 (c 0.78, DMSO-d6)

HR-MS (ESI) calc'd for M+Na⁺: 401.1723. found: 401.1741.

¹H-NMR (200 MHz, CDCl₃): δ 2.37 (quint, J=6.9 Hz, 1H), 2.64-2.98 (m, 2H), 3.48-3.90 (m, 4H), 3.73 (s, 6H), 4.73 (d, J=7.4 Hz, 1H), 4.89 (t, J=4.4 Hz, 1H), 6.76-6.98 (m, 3H), 7.33-7.60 (m, 4H), 7.79 (d, J=7.3 Hz, 1H), 7.92 (m, 1H), 8.19 (m, 1H).

### Example 4

### 9-17: ((2S,3R,4R)-2-(3,4-dimethoxyphenyl)-4-(4-(trifluoromethyl)benzyl)tetrahydro-furan-3-yl)methanol (3005)

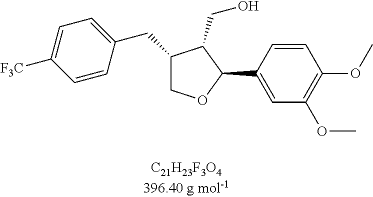 [SMILES: COc1ccc([C@H]2OC[C@H](Cc3ccc(C(F)(F)F)cc3)[C@@H]2CO)cc1OC]

Preparation identical to that of 9-12 except for using 1-bromo-4-(trifluoromethyl)benzene (55.6 mg, 0.247 mmol, 1.3 equiv.) as aryl halide coupling partner.

Work-up and purification: the heterogeneous reaction content was filtered and rinsed with CH₂Cl₂ (20 mL), the solvents evaporated and the target compound purified by flash column chromatography (18 g silica, flow rate 20 mL/min, gradient 15 to 80% EtOAc in LP within 45 min) to give a nearly colorless oil (57.2 mg, 76%).

[α]_{D}²⁵: +18.7 (c 2.68, MeOH)

HR-MS (ESI) calc'd for M+Na⁺: 419.1441. found: 419.1437.

¹H-NMR (200 MHz, CDCl₃): δ 1.79 (bs, 1H), 2.42 (quint, J=6.7 Hz, 1H), 2.60-2.86 (m, 2H), 3.04 (d, J=11.4 Hz, 1H), 3.71 (dd, J=8.6, 5.9 Hz, 1H), 3.76-3.97 (m, 2H), 3.86 (s, 3H), 3.87 (s, 3H), 4.03 (dd, J=8.6, 6.2 Hz, 1H), 4.82 (d, J=6.4 Hz, 1H), 6.78-6.91 (m, 3H), 7.31 (d, J=8.0 Hz, 2H), 7.55 (d, J=8.2 Hz, 2H).

### Comparative Example 7

### 9-18: ((2S,3R,4R)-2-(3,4-dimethoxyphenyl)-4-(3,4,5-trimethoxybenzyl)tetrahydro-furan-3-yl)methanol

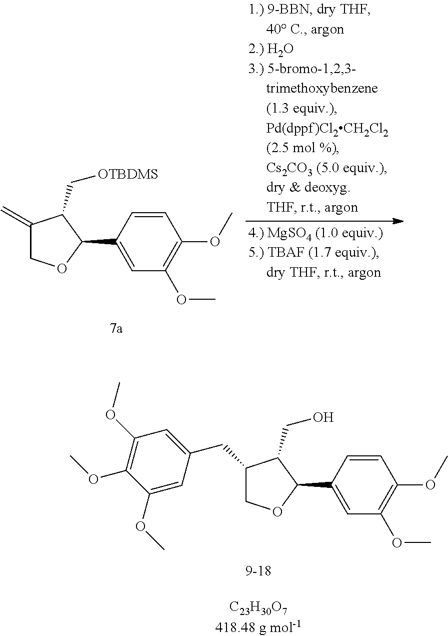

Preparation: a reaction vessel was charged with a stir bar and crude starting material 7a (658.7 mg, 1.807 mmol), and then evacuated and back-filled with argon using standard Schlenk technique (3×). Then was added a solution of 9-BBN (0.5 M in THF, 5.42 mL, 2.71 mmol) via syringe, the reaction stirred for 35 h at 40° C. and then allowed to cool to room temperature. Water (33 μL, 1.8 mmol) was subsequently added and stirring continued for 1 h to decompose excess 9-BBN.

This mixture was then purged by bubbling argon into the solution through a needle, and then was added dry and deoxygenated THF to produce a total volume of 7.0 mL, i.e. a 0.258 M solution of borylated intermediate, thus allowing the use of aliquots for subsequent coupling.

An aliquot (0.60 mL, 0.16 mmol, 1.0 equiv.) of this solution was then transferred via syringe to a separate vessel which had been charged with a stir bar, 5-bromo-1,2,3-trimethoxybenzene (49.8 mg, 0.202 mmol, 1.3 equiv.), Pd(dppf)Cl₂.CH₂Cl₂ (3.2 mg, 3.9 μmol, 2.5 mol %) and Cs₂CO₃ (253 mg, 0.775 mmol, 5.0 equiv.) under argon and then stirred for 37 h at room temperature. Following this, MgSO₄ (19 mg, 0.16 mmol, 1.0 equiv.) was added and stirring continued for 2 h to remove residual water. For deprotection, a solution of TBAF (1.0 M in THF, 0.26 mL, 0.26 mmol, 1.7 equiv.) was added via syringe and the mixture finally stirred for 12 h at room temperature.

Work-up and purification: the heterogeneous reaction content was filtered and rinsed with CH₂Cl₂ (20 mL), the solvents evaporated and the target compound purified first by flash column chromatography (18 g silica, flow rate 20 mL/min, gradient 30 to 100% EtOAc in LP within 50 min) and then preparative HPLC (Phenomenex® Luna 10 u C18(2) 100 A, 250×21.20 mm, flow rate 20.0 mL/min, gradient 45 to 55% MeOH in water within 60 min) to give a colorless oil (26.7 mg, 41%).

[α]_{D}²³: +15.1 (c 1.31, iPrOH)

HR-MS (APCI-PI) calc'd for M+Na⁺: 441.1884. found: 441.1893.

¹H-NMR (200 MHz, CDCl₃): δ 1.60 (bs, 1H), 2.36-2.63 (m, 2H), 2.66-2.85 (m, 1H), 2.95 (dd, J=12.8, 4.5 Hz, 1H), 3.72-3.99 (m, 3H), 3.83 (s, 3H), 3.84 (s, 6H), 3.87 (s, 3H), 3.89 (s, 3H), 4.07 (dd, J=8.5, 6.3 Hz, 1H), 4.81 (d, J=6.7 Hz, 1H), 6.41 (s, 2H), 6.79-6.92 (m, 3H).

¹³C-NMR (50 MHz, CDCl₃): δ 34.1, 42.4, 52.7, 56.0, 56.0, 56.2, 60.9, 61.0, 72.9, 82.8, 105.7, 109.1, 111.1, 118.1, 135.5, 136.3, 136.5, 148.5, 149.2, 153.3.

### Example 5

### 9-20: ((2S,3R,4R)-4-(4-butylbenzyl)-2-(3,4-dimethoxyphenyl)tetrahydrofuran-3-yl)-methanol (3008)

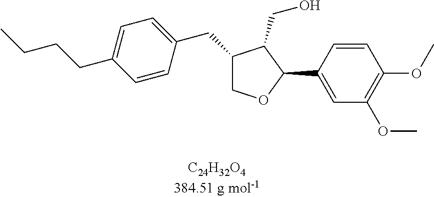 [SMILES: CCCCc1ccc(C[C@H]2CO[C@H](c3ccc(OC)c(OC)c3)[C@H]2CO)cc1]

Preparation identical to that of 9-18 except for using 1-bromo-4-butylbenzene (42.9 mg, 0.202 mmol, 1.3 equiv.) as aryl halide coupling partner.

Work-up and purification: the heterogeneous reaction content was filtered and rinsed with CH₂Cl₂ (20 mL), the solvents evaporated and the target compound purified by flash column chromatography (18 g silica, flow rate 20 mL/min, gradient 20 to 70% EtOAc in LP within 40 min) to give a nearly colorless oil (37.0 mg, 62%).

[α]_{D}²³: +4.7 (c 1.88, iPrOH)

HR-MS (APCI-PI) calc'd for M+Na⁺: 407.2193. found: 407.2194.

¹H-NMR (200 MHz, CDCl₃): δ 0.92 (t, J=7.2 Hz, 3H), 1.35 (sext, J=7.2 Hz, 2H), 1.48-1.69 (m, 3H), 2.40 (quint, J=6.7 Hz, 1H), 2.50-2.65 (m, 3H), 2.66-2.85 (m, 1H), 2.92 (dd, J=12.6, 4.8 Hz, 1H), 3.68-3.98 (m, 3H), 3.86 (s, 3H), 3.87 (s, 3H), 4.06 (dd, J=8.5, 6.4 Hz, 1H), 4.83 (d, J=6.3 Hz, 1H), 6.78-6.90 (m, 3H), 7.00-7.14 (m, 4H).

### Comparative Example 8

### 9-21: ((2S,3R,4R)-4-(3,5-dimethoxybenzyl)-2-(3,4-dimethoxyphenyl)tetrahydrofuran-3-yl)methanol

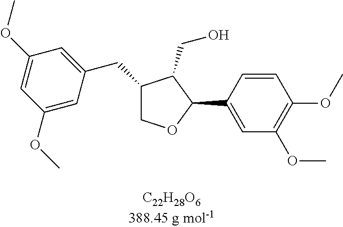 [SMILES: COc1cc(C[C@H]2CO[C@H](c3ccc(OC)c(OC)c3)[C@H]2CO)cc(OC)c1]

Preparation identical to that of 9-18 except for using 1-bromo-3,5-dimethoxybenzene 43.7 mg, 0.202 mmol, 1.3 equiv.) as aryl halide coupling partner.

Work-up and purification: the heterogeneous reaction content was filtered and rinsed with CH₂Cl₂ (20 mL), the solvents evaporated and the target compound purified by flash column chromatography (18 g silica, flow rate 20 mL/min, gradient 25 to 75% EtOAc in LP within 40 min) to give a pale brown oil (35.1 mg, 58%).

[α]_{D}²³: +13.6 (c 1.69, iPrOH)

HR-MS (APCI-PI) calc'd for M+Na⁺: 411.1778. found: 411.1756.

¹H-NMR (200 MHz, CDCl₃): δ 1.67 (bs, 1H), 2.41 (quint, J=6.8 Hz, 1H), 2.55 (dd, J=12.7, 10.1 Hz, 1H), 2.67-2.86 (m, 1H), 2.92 (dd, J=12.7, 4.8 Hz, 1H), 3.64-3.99 (m, 3H), 3.77 (s, 6H), 3.86 (s, 3H), 3.88 (s, 3H), 4.07 (dd, J=8.5, 6.4 Hz, 1H), 4.81 (d, J=6.4 Hz, 1H), 6.29-6.39 (m, 3H), 6.78-6.95 (m, 3H).

### Example 6

### 9-22: ((2S,3R,4R)-4-(4-(1,1-difluoroethyl)benzyl)-2-(3,4-dimethoxyphenyl)tetrahydro-furan-3-yl)methanol (3013)

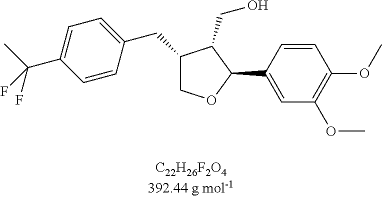 [SMILES: COc1ccc([C@H]2OC[C@H](Cc3ccc(C(C)(F)F)cc3)[C@@H]2CO)cc1OC]

Preparation identical to that of 9-18 except for using 1-bromo-4-(1,1-difluoroethyl)benzene (44.5 mg, 0.202 mmol, 1.3 equiv.) as aryl halide coupling partner.

Work-up and purification: the heterogeneous reaction content was filtered and rinsed with CH₂Cl₂ (20 mL), the solvents evaporated and the target compound purified by flash column chromatography (18 g silica, flow rate 20 mL/min, gradient 20 to 70% EtOAc in LP within 40 min) to give a pale brown oil (30.5 mg, 50%).

[α]_{D}²³: +10.4 (c 1.52, iPrOH)

HR-MS (APCI-PI) calc'd for M+Na⁺: 415.1691. found: 415.1667.

¹H-NMR (200 MHz, CDCl₃): δ 1.65 (bs, 1H), 1.91 (t, J=18.1 Hz, 3H), 2.41 (quint, J=6.7 Hz, 1H), 2.56-2.90 (m, 2H), 3.00 (dd, J=12.2, 3.7 Hz, 1H), 3.63-3.99 (m, 3H), 3.86 (s, 3H), 3.88 (s, 3H), 4.05 (dd, J=8.6, 6.3 Hz, 1H), 4.83 (d, J=6.3 Hz, 1H), 6.78-6.94 (m, 3H), 7.24 (d, J=7.9 Hz, 2H), 7.43 (d, J=8.2 Hz, 2H).

### Comparative Examples 9 to 11

Within this second group of examples, intermediate 7e, with R₄=R₆=H and R₅=F, was derivatized.

### Comparative Example 9

### 9-24: ((2S,3R,4R)-4-(3,4-dimethoxybenzyl)-2-(4-fluorophenyl)tetrahydrofuran-3-yl)-methanol

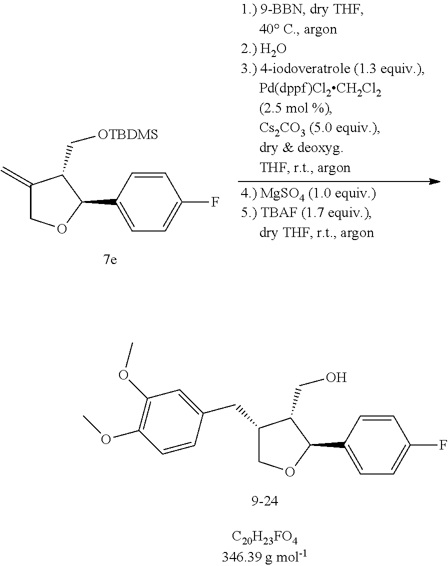 [SMILES: C=C1CO[C@H](c2ccc(F)cc2)[C@H]1CO[Si](C)(C)C(C)(C)C ; COc1ccc(C[C@H]2CO[C@H](c3ccc(F)cc3)[C@H]2CO)cc1OC]

Preparation: a reaction vessel was charged with a stir bar and crude starting material 7e (89%, 169.3 mg, 0.467 mmol), and then evacuated and back-filled with argon using standard Schlenk technique (3×). Then was added a solution of 9-BBN M in THF, 1.40 mL, 0.70 mmol) via syringe, the reaction stirred for 19 h at 40° C. and then allowed to cool to room temperature. Water (9 μL, 0.5 mmol) was subsequently added and stirring continued for 15 min to decompose excess 9-BBN.

This mixture was then purged by bubbling argon into the solution through a needle, and then was added dry and deoxygenated THF to produce a total volume of 2.7 mL, i.e. a 0.173 M solution of borylated intermediate, thus allowing the use of aliquots for subsequent coupling.

An aliquot (0.90 mL, 0.156 mmol, 1.0 equiv.) of this solution was then transferred via syringe to a separate vessel which had been charged with a stir bar, 4-iodoveratrole (53.5 mg, 0.202 mmol, 1.3 equiv.), Pd(dppf)Cl₂.CH₂Cl₂ (3.2 mg, 3.9 μmol, 2.5 mol %) and Cs₂CO₃ (254 mg, 0.779 mmol, 5.0 equiv.) under argon and then stirred for 19.5 h at room temperature. Following this, MgSO₄ (19 mg, 0.16 mmol, 1.0 equiv.) was added and stirring continued for 1.5 h to remove residual water. For deprotection, a solution of TBAF (1.0 M in THF, 0.26 mL, 0.26 mmol, 1.7 equiv.) was added via syringe and the mixture finally stirred for 21 h at room temperature.

Work-up and purification: the heterogeneous reaction content was filtered and rinsed with CH₂Cl₂ (20 mL), the solvents evaporated and the target compound purified by flash column chromatography (18 g silica, flow rate 20 mL/min, gradient 25 to 70% EtOAc in LP within 40 min) to give a yellow-orange oil (31.5 mg, 58%).

[α]_{D}²⁰: +10.4 (c 0.69, MeOH)

HR-MS (APCI-PI) calc'd for M+H+: 347.1653. found: 347.1668.

¹H-NMR (200 MHz, CDCl₃): δ 1.46 (bs, 1H, OH), 2.38 (quint, J=6.8 Hz, 1H), 2.56 (dd, J=12.6, 10.2 Hz, 1H), 2.65-2.83 (m, 1H), 2.92 (dd, J=12.7, 4.7 Hz, 1H), 3.77 (dd, J=8.6, 6.2 Hz, 1H), 3.86 (s, 3H), 3.86 (s, 3H), 3.79-4.00 (m, 2H), 4.07 (dd, J=8.5, 6.3 Hz, 1H), 4.87 (d, J=6.2 Hz, 1H), 6.68-6.84 (m, 3H), 6.95-7.08 (m, 2H), 7.24-7.35 (m, 2H).

¹³C-NMR (50 MHz, CDCl₃): δ 33.1, 42.4, 52.8, 56.0, 56.0, 60.7, 73.1, 82.4, 111.4, 112.0, 115.3, (d, J=21.4 Hz), 120.5, 127.4 (d, J=8.1 Hz), 133.0, 139.0 (d, J=3.0 Hz), 147.5, 149.0, 162.2 (d, J=245.4 Hz).

### Comparative Example 10

### 9-25: ((2S,3R,4R)-2-(4-fluorophenyl)-4-(4-methoxybenzyl)tetrahydrofuran-3-yl)-methanol

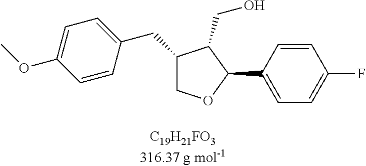 [SMILES: COc1ccc(C[C@H]2CO[C@H](c3ccc(F)cc3)[C@H]2CO)cc1]

Preparation identical to that of 9-24 except for using 4-bromoanisole (37.8 mg, 0.202 mmol, 1.3 equiv.) as aryl halide coupling partner.

Work-up and purification: the heterogeneous reaction content was filtered and rinsed with CH₂Cl₂ (20 mL), the solvents evaporated and the target compound purified by flash column chromatography (18 g silica, flow rate 20 mL/min, gradient 15 to 65% EtOAc in LP within 35 min) to give a light brown oil (28.1 mg, 58%).

[α]_{D}²³: +8.3 (c 1.08, MeOH)

¹H-NMR (200 MHz, CDCl₃): δ 1.46 (bs, 1H), 2.35 (quint, J=6.5 Hz, 1H), 2.57 (dd, J=12.5, 9.9 Hz, 1H), 2.61-2.81 (m, 1H), 2.89 (dd, J=12.5, 4.6 Hz, 1H), 3.69-3.83 (m, 2H), 3.79 (s, 3H), 3.93 (dd, J=10.6, 6.9 Hz, 1H), 4.06 (dd, J=8.6, 6.4 Hz, 1H), 4.89 (d, J=6.0 Hz, 1H), 6.79-6.88 (m, 2H), 6.96-7.15 (m, 4H), 7.24-7.34 (m, 2H).

### Comparative Example 11

### 9-26: ((2S,3R,4R)-4-(4-fluorobenzyl)-2-(4-fluorophenyl)tetrahydrofuran-3-yl)methanol

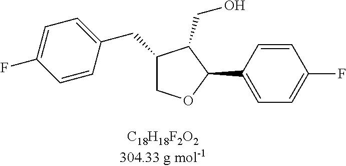 [SMILES: OC[C@H]1[C@@H](Cc2ccc(F)cc2)CO[C@@H]1c1ccc(F)cc1]

Preparation identical to that of 9-24 except for using 1-bromo-4-fluorobenzene (35.4 mg, 0.202 mmol, 1.3 equiv.) as aryl halide coupling partner.

Work-up and purification: the heterogeneous reaction content was filtered and rinsed with CH₂Cl₂ (20 mL), the solvents evaporated and the target compound purified by flash column chromatography (18 g silica, flow rate 20 mL/min, gradient 10 to 60% EtOAc in LP within 35 min) to give a light brown oil (22.6 mg, 48%).

[α]_{D}²³: +12.9 (c 0.77, MeOH)

¹H-NMR (200 MHz, CDCl₃): δ 1.47 (bs, 1H), 2.36 (quint, J=6.5 Hz, 1H), 2.52-2.81 (m, 2H), 2.94 (dd, J=12.3, 4.0 Hz, 1H), 3.66-3.98 (m, 3H), 4.05 (dd, J=8.6, 6.3 Hz, 1H), 4.88 (d, J=6.0 Hz, 1H), 6.91-7.08 (m, 4H), 7.09-7.19 (m, 2H), 7.26-7.34 (m, 2H).

### Comparative Example 12

In this example, intermediate 7c, with R₄=R₆=H and R₅=OCH₃, was derivatized.

### 9-27: ((2S,3R,4R)-4-(3,4-dimethoxybenzyl)-2-(4-methoxyphenyl)tetrahydrofuran-3-yl)methanol

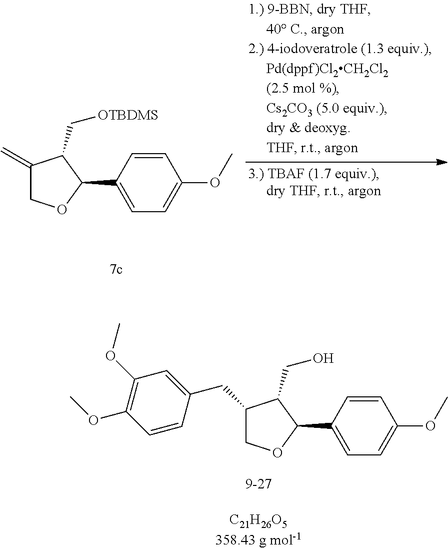

Preparation: a reaction vessel was charged with a stir bar and crude starting material 7c (137.2 mg, 0.410 mmol), and then evacuated and back-filled with argon using standard Schlenk technique (3×). Then was added a solution of 9-BBN (0.5 M in THF, 1.25 mL, 0.61 mmol) via syringe, the reaction stirred for 18 h at 40° C. and then allowed to cool to room temperature.

This mixture was then purged by bubbling argon into the solution through a needle, and then was added dry and deoxygenated THF to produce a total volume of 3.0 mL, i.e. a 0.137 M solution of borylated intermediate, thus allowing the use of aliquots for subsequent coupling.

An aliquot (1.50 mL, 0.21 mmol, 1.0 equiv.) of this solution was then transferred via syringe to a separate vessel which had been charged with a stir bar, 4-iodoveratrole (70.4 mg, 0.267 mmol, 1.3 equiv.), Pd(dppf)Cl₂.CH₂Cl₂ (4.2 mg, 5.1 μmol, 2.5 mol %) and Cs₂CO₃ (334 mg, 1.025 mmol, 5.0 equiv.) under argon and then stirred for 77 h at room temperature. For deprotection, a solution of TBAF (1.0 M in THF, 0.35 mL, 0.35 mmol, 1.7 equiv.) was added via syringe and the mixture finally stirred for 14 h at room temperature.

Work-up and purification: the heterogeneous reaction content was filtered and rinsed with CH₂Cl₂ (20 mL), the solvents evaporated and the target compound purified twice by flash column chromatography (18 g silica, flow rate 20 mL/min, gradient 30 to 80% EtOAc in LP within 40 min) to give a nearly colorless oil (21.4 mg, 29%).

[α]_{D}²⁵: +11.8 (c 1.14, iPrOH)

HR-MS (APCI-PI) calc'd for M+Na⁺: 381.1672. found: 381.1661.

¹H-NMR (200 MHz, CDCl₃): δ 1.55 (bs, 1H), 2.40 (quint, J=6.8 Hz, 1H), 2.55 (dd, J=12.6, 10.5 Hz, 1H), 2.65-2.84 (m, 1H), 2.93 (dd, J=12.8, 4.6 Hz, 1H), 3.68-3.98 (m, 3H), 3.79 (s, 3H), 3.86 (s, 6H), 4.05 (dd, J=8.5, 6.4 Hz, 1H), 4.80 (d, J=6.6 Hz, 1H), 6.68-6.83 (m, 3H), 6.87 (d, J=8.6 Hz, 2H), 7.25 (d, J=8.7 Hz, 2H).

¹³C-NMR (50 MHz, CDCl₃): δ 33.3, 42.5, 52.8, 55.4, 56.0, 56.0, 61.0, 73.0, 82.7, 111.4, 112.0, 114.0, 120.6, 127.2, 133.1, 135.1, 147.5, 149.1, 159.1.

### Examples 7 to 9 and Comparative Examples 13 to 17

Within this group, intermediate 7b, with R₄=R₅=R₆=OCH₃, was derivatized.

### Example 7

### 9-28: ((2S,3R,4R)-4-benzyl-2-(3,4,5-trimethoxyphenyl)tetrahydrofuran-3-yl)methanol (2756)

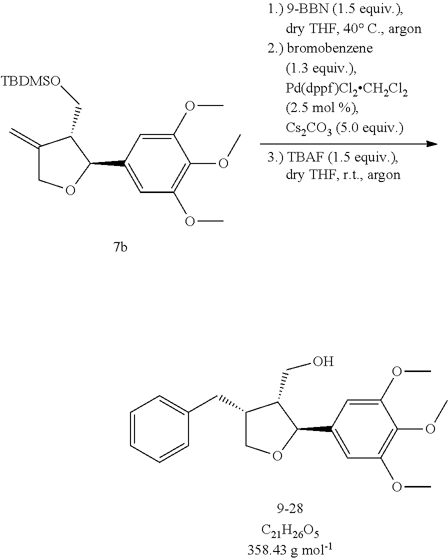

Preparation: a reaction vessel was charged with a stir bar and crude starting material 7b (52.0 mg, 0.132 mmol, 1.0 equiv.), and then evacuated and back-filled with argon using standard Schlenk technique (3×). Then was added a solution of 9-BBN (0.5 M in THF, 0.40 mL, 0.20 mmol) via syringe, the reaction stirred for 24 h at 40° C. and then allowed to cool to room temperature. The solution was transferred via syringe to a separate vessel which had been charged with a stir bar, bromobenzene (26.9 mg, 0.172 mmol, 1.3 equiv.), Pd(dppf)Cl₂.CH₂Cl₂ (2.7 mg, 3.3 μmol, 2.5 mol %) and Cs₂CO₃ (215 mg, 0.66 mmol, 5.0 equiv.) under argon and then stirred for 27 h at room temperature. For deprotection, a solution of TBAF (1.0 M in THF, 0.20 mL, 0.20 mmol, 1.5 equiv.) was added via syringe and the mixture finally stirred for 22 h at room temperature.

Work-up and purification: to the heterogeneous reaction content was added Et₂O and water, the layers separated and the aqueous phase extracted with Et₂O (4×) and EtOAc (2×). The combined organic phases were dried with Na₂SO₄, filtered and the solvents evaporated. The target compound was purified by flash column chromatography (9 g silica, flow rate 20 mL/min, gradient 15 to 70% EtOAc in LP within 45 min) to give an orange oil (22.2 mg, 36%).

[α]_{D}²⁰: +7.8 (c 1.96, MeOH)

HR-MS (ESI) calc'd for M+Na⁺: 381.1672. found: 381.1667.

¹H-NMR (200 MHz, CDCl₃): δ 1.91 (bs, 1H), 2.31-3.01 (m, 4H), 3.81 (s, 3H), 3.84 (s, 6H), 4.85 (d, J=5.9 Hz, 1H), 6.54 (s, 2H), 7.12-7.35 (m, 5H).

¹³C-NMR (50 MHz, CDCl₃): δ 33.5, 42.1, 52.4, 56.1, 60.8, 60.8, 73.0, 83.0, 102.5, 126.2, 128.6, 138.8, 140.4, 153.3. One C_{q} not visible.

### Comparative Example 13

### 9-29: ((2S,3R,4R)-4-(4-(tert-butyl)benzyl)-2-(3,4,5-trimethoxyphenyl)tetrahydrofuran-3-yl)methanol (2757)

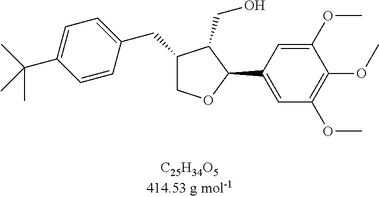 [SMILES: COc1cc([C@H]2OC[C@H](Cc3ccc(C(C)(C)C)cc3)[C@@H]2CO)cc(OC)c1OC]

Preparation analogous to that of 9-28, using crude starting material 7b (57.7 mg, 0.206 mmol, 1.0 equiv.) and 1-bromo-4-(tert-butyl)benzene (57.1 mg, 0.268 mmol, 1.3 equiv.) as aryl halide coupling partner.

Work-up and purification: to the heterogeneous reaction content was added Et₂O and water, the layers separated and the aqueous phase extracted with Et₂O (4×) and EtOAc (2×). The combined organic phases were dried with Na₂SO₄, filtered and the solvents evaporated. The target compound was purified by flash column chromatography (9 g silica, flow rate 20 mL/min, gradient 15 to 70% EtOAc in LP within 45 min) to give a nearly colorless oil (41.6 mg, 49%).

[α]_{D}²⁰: +11.0 (c 1.70, MeOH)

¹H-NMR (200 MHz, CDCl₃): δ 1.30 (s, 9H), 2.32-2.48 (m, 1H), 2.58 (dd, J=12.4, 9.6 Hz, 1H), 2.68-2.82 (m, 1H), 2.91 (dd, J=12.4, 4.9 Hz, 1H), 3.75 (dd, J=8.6, 6.8 Hz, 1H), 3.81 (s, 3H), 3.84 (s, 6H), 3.93 (dd, J=10.5, 6.8 Hz, 1H), 3.93 (dd, J=8.6, 6.4 Hz, 1H), 4.85 (d, J=5.7 Hz, 1H), 6.55 (s, 2H), 7.11 (d, J=8.3 Hz, 2H), 7.30 (d, J=8.3 Hz, 2H).

¹³C-NMR (50 MHz, CDCl₃): δ 31.3, 32.9, 34.3, 42.0, 52.3, 56.1, 60.8, 60.9, 73.1, 83.0, 102.5, 145.4, 128.2, 137.2, 138.9, 149.0, 153.2. One C_{q} not visible.

### Comparative Example 14

### 9-30: ((2S,3R,4R)-4-(4-methoxybenzyl)-2-(3,4,5-trimethoxyphenyl)tetrahydrofuran-3-yl)methanol

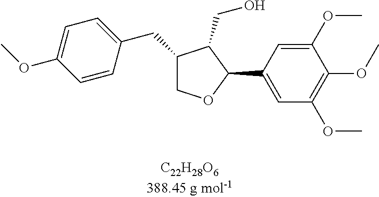

Preparation analogous to that of 9-28, using crude starting material 7b (52.0 mg, 0.132 mmol, 1.0 equiv.) and 4-bromoanisole (32.1 mg, 0.172 mmol, 1.3 equiv.) as aryl halide coupling partner.

Work-up and purification: to the heterogeneous reaction content was added Et₂O and water, the layers separated and the aqueous phase extracted with Et₂O (4×) and EtOAc (2×). The combined organic phases were dried with Na₂SO₄, filtered and the solvents evaporated. The target compound was purified by flash column chromatography (9 g silica, flow rate 20 mL/min, gradient 15 to 70% EtOAc in LP within 45 min) to give an orange oil (19.3 mg, 28%).

[α]_{D}²⁰: +3.4 (c 0.72, MeOH)

HR-MS (ESI) calc'd for M+Na⁺: 411.1778. found 411.1785.

¹H-NMR (200 MHz, CDCl₃): δ 1.54 (bs, 1H), 2.31-2.96 (m, 4H), 3.76-3.99 (m, 3H), 3.78 (s, 3H), 3.82 (s, 3H), 3.84 (s, 6H), 4.00-4.11 (m, 1H), 4.84 (d, J=5.9 Hz, 1H), 6.54 (s, 2H), 6.77-6.78 (m, 2H), 7.04-7.15 (m, 2H).

### Example 8

### 9-32: ((2S,3R,4R)-4-(4-(trifluoromethyl)benzyl)-2-(3,4,5-trimethoxyphenyl)tetrahydro-furan-3-yl)methanol (2760)

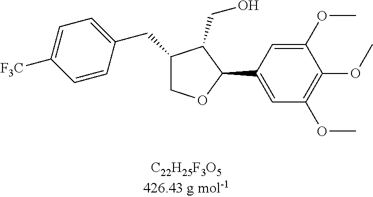 [SMILES: COc1cc([C@H]2OC[C@H](Cc3ccc(C(F)(F)F)cc3)[C@@H]2CO)cc(OC)c1OC]

Preparation analogous to that of 9-28, using crude starting material 7b (52.0 mg, 0.132 mmol, 1.0 equiv.) and 1-bromo-4-(trifluoromethyl)benzene (38.6 mg, 0.172 mmol, 1.3 equiv.) as aryl halide coupling partner.

Work-up and purification: to the heterogeneous reaction content was added Et₂O and water, the layers separated and the aqueous phase extracted with Et₂O (4×) and EtOAc (2×). The combined organic phases were dried with Na₂SO₄, filtered and the solvents evaporated. The target compound was purified by flash column chromatography (9 g silica, flow rate 20 mL/min, gradient 15 to 70% EtOAc in LP within 45 min) to give a slightly yellow oil (40.2 mg, 62%).

[α]_{D}²⁰: +15.5 (c 0.1.72, MeOH)

HR-MS (ESI) calc'd for M+Na⁺:449.1546. found: 449.1551.

¹H-NMR (200 MHz, CDCl₃): δ 1.74 (bs, 1H), 2.33-2.49 (m, 1H), 2.57-3.10 (m, 3H), 3.63-4.10 (m, 4H), 3.81 (s, 3H), 3.84 (s, 6H), 4.83 (d, J=5.9 Hz, 1H), 6.54 (s, 2H), 7.30 (d, J=8.0 Hz, 2H), 7.54 (d, J=8.0 Hz, 2H).

### Comparative Example 15

### 9-33: ((2S,3R,4R)-4-(4-methylbenzyl)-2-(3,4,5-trimethoxyphenyl)tetrahydrofuran-3-yl)methanol

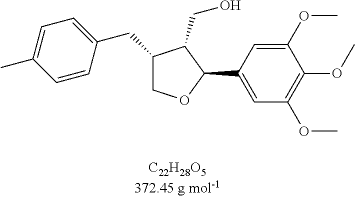

Preparation analogous to that of 9-28, using crude starting material 7b (57.7 mg, 0.206 mmol, 1.0 equiv.) and 1-iodotoluene (58.4 mg, 0.268 mmol, 1.3 equiv.) as aryl halide coupling partner.

Work-up and purification: to the heterogeneous reaction content was added Et₂O and water, the layers separated and the aqueous phase extracted with Et₂O (4×) and EtOAc (2×). The combined organic phases were dried with Na₂SO₄, filtered and the solvents evaporated. The target compound was purified by flash column chromatography (9 g silica, flow rate 20 mL/min, gradient 15 to 70% EtOAc in LP within 45 min) to give a slightly yellow oil (33.2 mg, 43%).

[α]_{D}²⁰: +12.3 (c 1.22, MeOH)

HR-MS (ESI) calc'd for M+Na⁺: 395.1829. found: 395.1829.

¹H-NMR (200 MHz, CDCl₃): δ 1.73 (bs, 1H), 2.31 (s, 3H), 2.33-2.79 (m, 4H), 2.83-2.98 (dd, J=12.5, 4.7 Hz, 1H), 3.68-3.98 (m, 3H), 3.81 (s, 3H), 3.84 (s, 6H), 4.00-4.11 (m, 1H), 4.84 (d, J=5.7 Hz, 1H), 6.54 (s, 2H), 7.08 (s, 4H).

### Example 9

### 9-34: ((2S,3R,4R)-4-([1,1′-biphenyl]-4-ylmethyl)-2-(3,4,5-trimethoxyphenyl)-tetrahydrofuran-3-yl)methanol (2767)

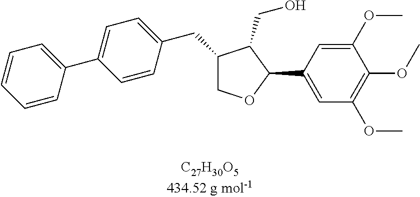 [SMILES: COc1cc([C@H]2OC[C@H](Cc3ccc(-c4ccccc4)cc3)[C@@H]2CO)cc(OC)c1OC]

Preparation analogous to that of 9-28, using crude starting material 7b (57.7 mg, 0.206 mmol, 1.0 equiv.) and 4-bromo-1,1′-biphenyl (62.4 mg, 0.268 mmol, 1.3 equiv.) as aryl halide coupling partner.

Work-up and purification: to the heterogeneous reaction content was added Et₂O and water, the layers separated and the aqueous phase extracted with Et₂O (4×) and EtOAc (2×). The combined organic phases were dried with Na₂SO₄, filtered and the solvents evaporated. The target compound was purified by flash column chromatography (9 g silica, flow rate 20 mL/min, gradient 15 to 70% EtOAc in LP within 45 min) to give a yellow oil (40.3 mg, 45%).

[α]_{D}²⁰: +7.1 (c 1.72, MeOH)

¹H-NMR (200 MHz, CDCl₃): δ 1.78 (bs, 1H), 2.35-2.52 (m, 1H), 2.58-2.90 (m, 2H), 2.93-3.06 (m, 1H), 3.72-3.89 (m, 3H), 3.82 (s, 3H), 3.85 (s, 6H), 3.91-4.02 (m, 1H), 4.87 (d, J=5.7 Hz, 1H), 6.56 (s, 2H), 7.19-7.64 (m, 9H).

¹³C-NMR (50 MHz, CDCl₃): δ 33.1, 42.0, 52.3, 56.1, 60.8, 60.9, 73.0, 83.0, 102.5, 126.9, 127.1, 127.2, 128.7, 129.0, 137.0, 138.9, 139.1, 139.4, 140.7, 153.2.

### Comparative Example 16

### 9-36: 1-(4-(((3R,4R,5S)-4-(hydroxymethyl)-5-(3,4,5-trimethoxyphenyl)tetrahydrofuran-3-yl)methyl)phenyl)ethanone

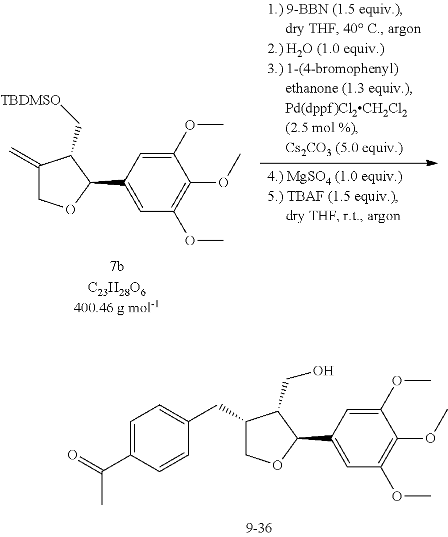

Preparation: a reaction vessel was charged with a stir bar and crude starting material 7b (57.7 mg, 0.206 mmol, 1.0 equiv.), and then evacuated and back-filled with argon using standard Schlenk technique (3×). Then was added a solution of 9-BBN (0.5 M in THF, 0.62 mL, 0.31 mmol) via syringe, the reaction stirred for 24 h at 40° C. and then allowed to cool to room temperature. Water (4 μL, 0.21 mmol, 1.0 equiv.) was subsequently added and stirring continued for 30 min to decompose excess 9-BBN, before the solution was transferred via syringe to a separate vessel which had been charged with a stir bar, 1-(4-bromophenyl)ethanone (53.3 mg, 0.268 mmol, 1.3 equiv.), Pd(dppf)Cl₂.CH₂Cl₂ (4.2 mg, 5.2 μmol, 2.5 mol %) and Cs₂CO₃ (336 mg, 1.03 mmol, 5.0 equiv.) under argon and then stirred for 27 h at room temperature. Following this, MgSO₄ (25 mg, 0.21 mmol, 1.0 equiv.) was added and stirring continued for 30 to remove residual water. For deprotection, a solution of TBAF (1.0 M in THF, 0.31 mL, 0.31 mmol, 1.5 equiv.) was added via syringe and the mixture finally stirred for 22 h at room temperature.

Work-up and purification: to the heterogeneous reaction content was added Et₂O and water, the layers separated and the aqueous phase extracted with Et₂O (4×) and EtOAc (2×). The combined organic phases were dried with Na₂SO₄, filtered and the solvents evaporated. The target compound was purified by flash column chromatography (9 g silica, flow rate 20 mL/min, gradient 15 to 70% EtOAc in LP within 45 min) to give a yellow oil (19.5 mg, 24%).

[α]_{D}²⁰: +10.9 (c 1.55, MeOH)

HR-MS (ESI) calc'd for M+Na⁺: 423.1778. found: 423.1785.

¹H-NMR (200 MHz, CDCl₃): δ 1.73 (bs, 1H), 2.30-2.59 (m, 1H), 2.65 (s, 3H), 2.59-2.83 (m, 2H), 2.96-3.08 (m, 1H), 3.64-3.95 (m, 3H), 3.80 (s, 3H), 3.83 (s, 6H), 3.97-4.06 (m, 1H), 4.82 (d, J=6.1 Hz, 1H), 6.53 (s, 2H), 7.28 (d, J=8.4 Hz, 2H), 7.88 (d, J=8.4 Hz, 2H).

¹³C-NMR (50 MHz, CDCl₃): δ 26.5, 36.3, 41.9, 52.3, 50.1, 60.8, 60.7, 72.7, 82.9, 102.5, 128.7, 128.8, 135.3, 137.1, 138.6, 146.3, 153.2, 197.8.

### Comparative Example 17

### 9-37: ethyl 4-(((3R,4R,5S)-4-(hydroxymethyl)-5-(3,4,5-trim ethoxyphenyl)tetrahydro-furan-3-yl)methyl)benzoate

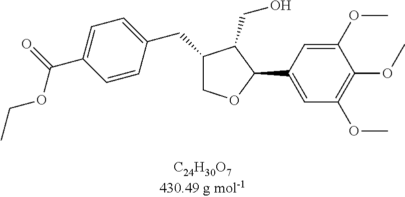 [SMILES: CCOC(=O)c1ccc(C[C@H]2CO[C@H](c3cc(OC)c(OC)c(OC)c3)[C@H]2CO)cc1]

Preparation analogous to that of 9-36, using crude starting material 7b (57.7 mg, 0.206 mmol, 1.0 equiv.) and ethyl 4-bromobenzoate (61.4 mg, 0.268 mmol, 1.3 equiv.) as aryl halide coupling partner.

Work-up and purification: to the heterogeneous reaction content was added Et₂O and water, the layers separated and the aqueous phase extracted with Et₂O (4×) and EtOAc (2×). The combined organic phases were dried with Na₂SO₄, filtered and the solvents evaporated. The target compound was purified by flash column chromatography (9 g silica, flow rate 20 mL/min, gradient 15 to 70% EtOAc in LP within 45 min) to give a slightly yellow oil (40.0 mg, 45%).

[α]_{D}²⁰: +7.7 (c 2.02, MeOH)

HR-MS (ESI) calc'd for M+Na⁺: 453.1884. found: 453.1886.

¹H-NMR (400 MHz, CDCl₃): δ 1.39 (t, J=7.4 Hz, 3H), 1.94 (bs, 1H), 2.38-2.46 (m, 1H), 2.69 (dd, J=12.9, 11.0 Hz, 1H), 2.72-2.82 (m, 1H), 3.07 (dd, J=8.6, 4.3 Hz, 1H), 3.72 (dd, J=8.2, 6.7 Hz, 1H), 3.83 (s, 3H), 3.85 (s, 6H), 3.92 (dd, J=10.4, 6.8 Hz, 1H), 4.04 (dd, J=8.6, 6.7 Hz, 1H), 4.37 (q, J=7.4 Hz, 2H), 4.85 (d, J=5.9, 1H), 6.55 (s, 2H), 7.27 (d, J=8.2 Hz, 2H), 7.97 (d, J=8.2 Hz, 2H).

### Comparative Examples 18 to 20

Within this group, intermediate 7d, with R₄=R₅=R₆=H. was derivatized.

### Comparative Example 18

### 9-39: ((2S,3R,4R)-4-(3,4-dimethoxybenzyl)-2-phenyltetrahydrofuran-3-yl)methanol

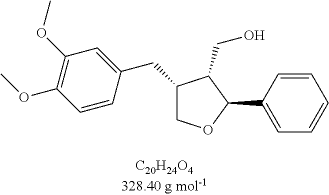 [SMILES: COc1ccc(C[C@H]2CO[C@H](c3ccccc3)[C@H]2CO)cc1OC]

Preparation analogous to that of 9-36, using crude starting material 7d (81%, 54.8 mg, 0.146 mmol, 1.0 equiv.) and 4-iodoveratrole (50.1 mg, 0.190 mmol, 1.3 equiv.) as aryl halide coupling partner, and stirring for 42 in place of 27 h during the coupling step.

Work-up and purification: the heterogeneous reaction content was filtered and rinsed with CH₂Cl₂ (20 mL), the solvents evaporated and the target compound purified by flash column chromatography (18 g silica, flow rate 20 mL/min, gradient 15 to 40% EtOAc in LP within 35 min) to give a colorless oil (24.5 mg, 51%).

[α]_{D}²⁰: +10.1 (c 7.33, MeOH)

HR-MS (ESI) calc'd for M+Na⁺: 351.1567. found: 351.1569.

¹H-NMR (200 MHz, CDCl₃): δ 1.71 (bs, 1H), 2.41 (quint, J=6.6 Hz, 1H), 2.56 (dd, J=12.7, 10.3 Hz, 1H), 2.64-2.83 (m, 1H), 2.92 (dd, J=12.7, 4.6 Hz, 1H), 3.78 (dd, J=8.5, 6.2 Hz, 1H), 3.86 (s, 6H), 3.81-3.99 (m, 2H), 4.08 (dd, J=8.5, 6.4 Hz, 1H), 4.89 (d, J=6.2 Hz, 1H), 6.66-6.83 (m, 3H), 7.20-7.40 (m, 5H).

### Comparative Example 19

### 9-40: ((2S,3R,4R)-4-(4-methoxybenzyl)-2-phenyltetrahydrofuran-3-yl)methanol

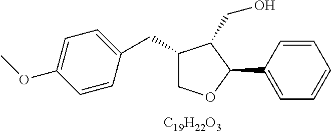

Preparation analogous to that of 9-36, using crude starting material 7d (81%, 56.3 mg, 0.150 mmol, 1.0 equiv.) and 4-bromoanisole (36.4 mg, 0.195 mmol, 1.3 equiv.) as aryl halide coupling partner, and stirring for 42 in place of 27 h during the coupling step.

Work-up and purification: the heterogeneous reaction content was filtered and rinsed with CH₂Cl₂ (20 mL), the solvents evaporated and the target compound purified by flash column chromatography (18 g silica, flow rate 20 mL/min, gradient 10 to 40% EtOAc in LP within 35 min) to give a colorless oil (32.7 mg, 73%).

[α]_{D}²³: +12.8 (c 1.40, MeOH)

¹H-NMR (200 MHz, CDCl₃): δ 1.71 (bs, 1H), 2.40 (quint, J=6.4 Hz, 1H), 2.57 (dd, J=12.5, 10.0 Hz, 1H), 2.64-2.82 (m, 1H), 2.90 (dd, J=12.5, 4.6 Hz, 1H), 3.70-3.84 (m, 2H), 3.78 (s, 3H), 3.93 (dd, J=10.8, 6.9 Hz, 1H), 4.08 (dd, J=8.5, 6.4 Hz, 1H), 4.90 (d, J=6.0 Hz, 1H), 6.78-6.88 (m, 2H), 7.06-7.15 (m, 2H), 7.21-7.39 (m, 5H).

### Comparative Example 20

### 9-41: ((2S,3R,4R)-2-phenyl-4-(4-(trifluoromethyl)benzyl)tetrahydrofuran-3-yl)-methanol

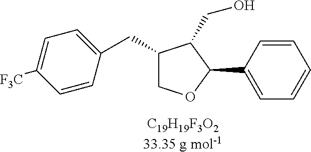

Preparation analogous to that of 9-36, using crude starting material 7d (81%, 44.5 mg, 0.118 mmol, 1.0 equiv.) and 1-bromo-4-(trifluoromethyl)benzene (34.6 mg, 0.154 mmol, 1.3 equiv.) as aryl halide coupling partner, and stirring for 42 in place of 27 h during the coupling step.

Work-up and purification: the heterogeneous reaction content was filtered and rinsed with CH₂Cl₂ (20 mL), the solvents evaporated and the target compound purified by flash column chromatography (18 g silica, flow rate 20 mL/min, gradient 10 to 40% EtOAc in LP within 35 min) to give a colorless oil (17.4 mg, 44%).

[α]_{D}²³: +15.3 (c 1.61, MeOH)

¹H-NMR (200 MHz, CDCl₃): δ 1.59 (bs, 1H), 2.42 (quint, J=6.4 Hz, 1H), 2.62-2.86 (m, 2H), 2.99-3.10 (m, 1H), 3.67-3.99 (m, 3H), 4.06 (dd, J=8.5, 6.2 Hz, 1H), 4.90 (d, J=6.1 Hz, 1H), 7.21-7.40 (m, 7H), 7.55 (d, J=8.1 Hz, 2H).

### Comparative Examples 21 and 22

Within this group of examples, intermediate 7e, with R₄=R₆=H and R₅=F, was again derivatized.

### Comparative Example 21

### 9-42: ((2S,3R,4R)-4-(4-(tert-butyl)benzyl)-2-(4-fluorophenyl)tetrahydrofuran-3-yl)-methanol

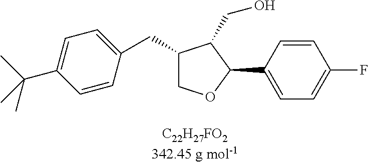 [SMILES: CC(C)(C)c1ccc(C[C@H]2CO[C@H](c3ccc(F)cc3)[C@H]2CO)cc1 ; "C22H27FO2342.45 g mol-1"]

Preparation analogous to that of 9-36, using crude starting material 7e (41.3 mg, 0.128 mmol, 1.0 equiv.) and 1-bromo-4-(tert-butyl)benzene (35.5 mg, 0.166 mmol, 1.3 equiv.) as aryl halide coupling partner, and stirring for 42 in place of 27 h during the coupling step.

Work-up and purification: the heterogeneous reaction content was filtered and rinsed with CH₂Cl₂ (20 mL), the solvents evaporated and the target compound purified by flash column chromatography (18 g silica, flow rate 20 mL/min, gradient 10 to 80% EtOAc in LP within 60 min) to give a colorless oil (13.5 mg, 31%).

[α]_{D}²³: +6.8 (c 1.07, MeOH) a

¹H-NMR (200 MHz, CDCl₃): δ 1.31 (s, 9H), 1.49 (bs, 1H), 2.36 (quint, J=6.5, 1 H), 2.52-2.96 (m, 3H), 3.69-3.84 (m, 2H), 3.93 (dd, J=10.8, 6.9 Hz, 1H), 4.09 (dd, J=8.6, 6.3 Hz, 1H), 4.90 (d, J=5.8 Hz, 1H), 6.95-7.15 (m, 4H), 7.26-7.35 (m, 4H).

### Comparative Example 22

### 9-43: ((2S,3R,4R)-2-(4-fluorophenyl)-4-(4-(trifluoromethyl)benzyl)tetrahydrofuran-3-yl)methanol

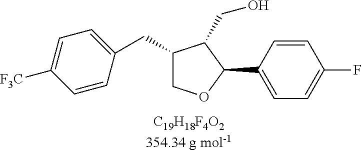

Preparation analogous to that of 9-36, using crude starting material 7e (39.0 mg, 0.121 mmol, 1.0 equiv.) and 1-bromo-4-(trifluoromethyl)benzene (35.4 mg, 0.157 mmol, 1.3 equiv.) as aryl halide coupling partner, and stirring for 42 in place of 27 h during the coupling step.

Work-up and purification: the heterogeneous reaction content was filtered and rinsed with CH₂Cl₂ (20 mL), the solvents evaporated and the target compound purified by flash column chromatography (18 g silica, flow rate 20 mL/min, gradient 15 to 40% EtOAc in LP within 35 min) to give a nearly colorless oil (17.5 mg, 41%).

[α]_{D}²³: +12.0 (c 1.36, MeOH)

¹H-NMR (200 MHz, CDCl₃): δ 1.84 (bs, 1H), 2.36 (quint, J=6.5 Hz, 1H), 2.59-2.84 (m, 2H), 3.02 (d, J=9.0 Hz, 1H), 3.65-3.95 (m, 3H), 4.03 (dd, J=8.5, 6.3 Hz, 1H), 4.87 (d, J=6.1 Hz, 1H), 6.94-7.07 (m, 2H), 7.21-7.34 (m, 4H), 7.54 (d, J=8.1 Hz, 2H).

### Examples 10 to 22 and Comparative Examples 23 to 34

In this and the two following groups of examples, free alcohols of formula (9) will finally be transformed through esterification or etherification, respectively, into compounds of formula (10) (with R₇≠OH). First, Mitsunobu esterifications are disclosed. Designation of the compounds obtained is analogous to those of formula (9), i.e., formula number 10, followed by another basically consecutively increasing number (with corresponding compounds within this row which are missing).

### Comparative Example 23

### 10-1: (Z)-((2S,3R,4R)-4-benzyl-2-(3,4-dimethoxyphenyl)tetrahydrofuran-3-yl)methyl 2-methylbut-2-enoate

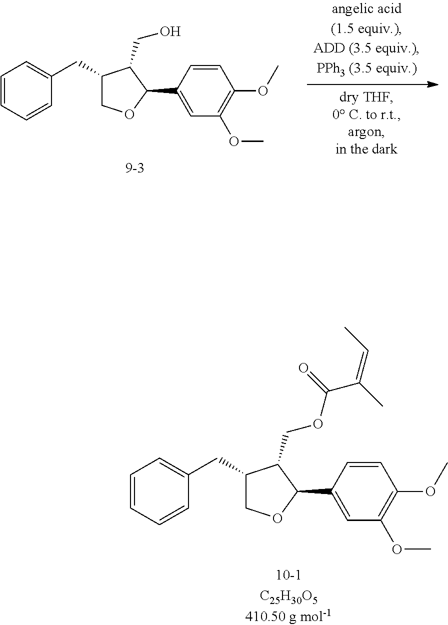 [SMILES: COc1ccc([C@H]2OC[C@H](Cc3ccccc3)[C@@H]2CO)cc1OC ; C/C=C(/C)C(=O)OC[C@H]1[C@@H](Cc2ccccc2)CO[C@@H]1c1ccc(OC)c(OC)c1]

Preparation: a reaction vessel was charged with a stir bar, starting material 9-3 (24.4 mg, 0.074 mmol, 1.0 equiv.), angelic acid (11.2 mg, 0.111 mmol, 1.5 equiv.) and PPh₃ (68.2 mg, 0.260 mmol, 3.5 equiv.), and then evacuated and back-filled with argon using standard Schlenk technique (3×). Dry THF (0.75 mL) was then added and the solution cooled to 0° C. in an ice bath. To the stirred mixture was then added a solution of ADD (65.6 mg, 0.260 mmol, 3.5 equiv.) in dry THF (1.0 mL) via syringe over approximately 1 min, and the reaction stirred for 22 h while being kept away from light and allowed to warm slowly to room temperature.

Work-up and purification: Et₂O (5 mL) was added to the reaction content, which was then filtered and rinsed with more Et₂O (15 mL) The solvents were evaporated and the target compound purified by flash column chromatography (9 g silica, flow rate 20 mL/min, gradient 1 to 35% EtOAc in LP within 30 min) to give a colorless oil (26.4 mg, 86%).

[α]_{D}²⁵: +31.6 (c 2.64, MeOH)

HR-MS (ESI) calc'd for M+Na⁺: 433.1985. found: 433.1968.

¹H-NMR (200 MHz, CDCl₃): δ 1.85-1.91 (m, 3H), 2.00 (dq, J=7.3, 1.5 Hz, 3H), 2.70-2.53 (m, 2H), 2.89-2.70 (m, 1H), 2.95 (dd, J=12.5, 4.1 Hz, 1H), 3.77 (dd, J=8.6, 6.3 Hz, 1H), 3.87 (s, 3H), 3.88 (s, 3H), 4.07 (dd, J=8.6, 6.2 Hz, 1H), 4.28 (dd, J=11.4, 7.0 Hz, 1H), 4.41 (dd, J=11.4, 7.0 Hz, 1H), 4.85 (d, J=6.3 Hz, 1H), 6.10 (qq, J=7.2, 1.4 Hz, 1H), 6.92-6.79 (m, 3H), 7.36-7.13 (m, 5H).

¹³C-NMR (50 MHz, CDCl₃): δ 16.0, 20.7, 33.7, 42.5, 49.3, 56.0, 56.1, 62.3, 72.8, 83.0, 108.9, 111.1, 118.1, 126.4, 127.5, 128.7, 128.7, 135.1, 139.1, 140.2, 148.6, 149.2, 167.8.

### Example 10

### 10-4: (Z)-((2S,3R,4R)-4-(4-(tert-butyl)benzyl)-2-(3,4-dimethoxyphenyl)tetrahydro-furan-3-yl)methyl 2-methylbut-2-enoate (2788)

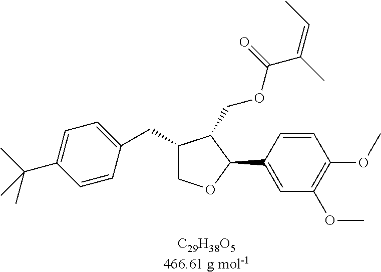 [SMILES: C/C=C(/C)C(=O)OC[C@H]1[C@@H](Cc2ccc(C(C)(C)C)cc2)CO[C@@H]1c1ccc(OC)c(OC)c1 ; "C29H38O5466.61 g mol-1"]

Preparation analogous to that of 10-1, using starting material 9-5 (31.1 mg, 0.081 mmol, 1.00 equiv.).

Work-up and purification: Et₂O (5 mL) was added to the reaction content, which was then filtered and rinsed with more Et₂O (15 mL) The solvents were evaporated and the target compound purified by flash column chromatography (9 g silica, flow rate 20 mL/min, isocratically at 7% EtOAc in LP for 3 min, then gradient 7 to 35% within 30 min) to give a colorless oil (25.4 mg, 67%).

[α]_{D}²⁰: +21.4 (c 1.52, MeOH)

HR-MS (APCI-PI) calc'd for M+Na⁺: 489.2611. found 489.2676.

¹H-NMR (200 MHz, CDCl₃): δ 1.31 (s, 9H), 1.85-1.90 (m, 3H), 2.00 (dq, J=7.2, 1.5 Hz, 3H), 2.53-2.86 (m, 3H), 2.91 (dd, J=12.4, 4.2 Hz, 1H), 3.80 (dd, J=8.5, 6.3 Hz, 1H), 3.87 (s, 3H), 3.88 (s, 3H), 4.09 (dd, J=8.6, 6.3 Hz, 1H), 4.28 (dd, J=11.3, 7.0 Hz, 1H), 4.42 (dd, J=11.4, 7.0 Hz, 1H), 4.84 (d, J=6.3 Hz, 1H), 6.10 (qq, J=7.2, 1.4 Hz, 1H), 6.79-6.92 (m, 3H), 7.10 (d, J=8.3 Hz, 2H), 7.32 (d, J=8.3 Hz, 2H).

### Example 11

### 10-8: ethyl 4-(((3R,4R,5S)-5-(3,4-dimethoxyphenyl)-4-((((Z)-2-methylbut-2-enoyl)-oxy)methyl)tetrahydrofuran-3-yl)methyl)benzoate (2792)

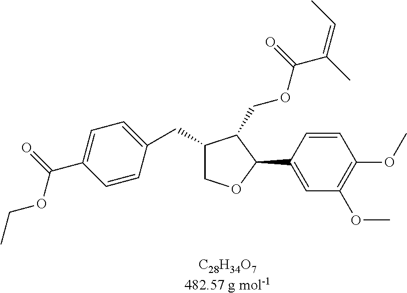 [SMILES: C/C=C(/C)C(=O)OC[C@H]1[C@@H](Cc2ccc(C(=O)OCC)cc2)CO[C@@H]1c1ccc(OC)c(OC)c1 ; "C28H34O7482.57 g mol-1"]

Preparation analogous to that of 10-1, using starting material 9-6 (26.7 mg, 0.067 mmol, 1.00 equiv.).

Work-up and purification: Et₂O (5 mL) was added to the reaction content, which was then filtered and rinsed with more Et₂O (15 mL) The solvents were evaporated and the target compound purified by flash column chromatography (9 g silica, flow rate 20 mL/min, gradient 5 to 40% EtOAc in LP within 30 min) to give a colorless oil (28.8 mg, 89%).

[α]_{D}²⁵: +18.4 (c 1.66, MeOH)

HR-MS (APCI-PI) calc'd for M+Na⁺: 505.2197. found: 505.2229.

¹H-NMR (200 MHz, CDCl₃): δ 1.39 (t, J=7.1 Hz, 3H), 1.88 (s, 3H), 2.00 (d, J=7.3 Hz, 3H), 2.55-2.90 (m, 3H), 2.94-3.06 (m, 1H), 3.74 (dd, J=8.7, 6.0 Hz, 1H), 3.87 (s, 3H), 3.87 (s, 3H), 4.06 (dd, J=8.7, 6.0 Hz, 1H), 4.20-4.46 (m, 4H), 4.84 (d, J=6.4 Hz, 1H), 6.11 (q, J=7.3 Hz, 1H), 6.79-6.91 (m, 3H), 7.25 (d, J=7.9 Hz, 2H), 7.98 (d, J=8.1 Hz, 2H).

### Example 12

### 10-11: (Z)-((2S,3R,4R)-2-(3,4-dimethoxyphenyl)-4-(pyridin-2-ylmethyl)tetrahydro-furan-3-yl)methyl 2-methylbut-2-enoate (2823)

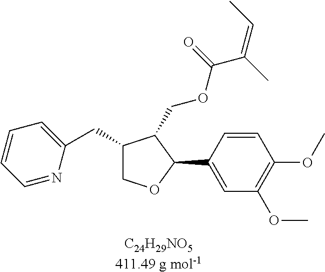 [SMILES: C/C=C(/C)C(=O)OC[C@H]1[C@@H](Cc2ccccn2)CO[C@@H]1c1ccc(OC)c(OC)c1 ; "C24H29NO5411.49 g mol-1"]

Preparation analogous to that of 10-1, using starting material 9-13 (21.9 mg, 0.066 mmol, 1.00 equiv.).

Work-up and purification: Et₂O (5 mL) was added to the reaction content, which was then filtered and rinsed with more Et₂O (15 mL) The solvents were evaporated and the target compound purified first by flash column chromatography g silica, flow rate 20 mL/min, gradient 20 to 50% EtOAc in LP within 40 min) and then by preparative HPLC (Phenomenex® Luna 10 u C18(2) 100 A, 250×21.20 mm, flow rate 20.0 mL/min, isocratically at 60% MeOH in water) to give a colorless oil (14.2 mg, 52%).

[α]_{D}²³: +33.0 (c 0.80, MeOH)

HR-MS (ESI) calc'd for M+H+: 412.2118. found: 412.2107.

¹H-NMR (200 MHz, CDCl₃): δ 1.82-1.89 (m, 3H), 1.98 (dd, J=7.2, 1.5 Hz, 3H), 2.64 (quint, J=6.7 Hz, 1H), 2.86 (dd, J=15.2, 11.9 Hz, 1H), 2.98-3.19 (m, 2H), 3.77 (dd, J=8.5, 6.9 Hz, 1H), 3.86 (s, 3H), 3.88 (s, 3H), 4.15 (dd, J=8.5, 6.5 Hz, 1H), 4.27 (dd, J=11.4, 7.0 Hz, 1H), 4.42 (dd, J=11.4, 6.7 Hz, 1H), 4.85 (d, J=6.2 Hz, 1H), 6.08 (qd, J=7.2, 1.3 Hz, 1H), 6.78-6.90 (m, 3H), 7.12-7.21 (m, 2H), 7.64 (td, J=7.7, 1.7 Hz, 1H), 8.56 (d, J=4.5 Hz, 1H).

### Comparative Example 24

### 10-13: (Z)-((2S,3R,4R)-2-(3,4-dimethoxyphenyl)-4-(4-(trifluoromethyl)benzyl)tetra-hydrofuran-3-yl)methyl 2-methylbut-2-enoate (2827)

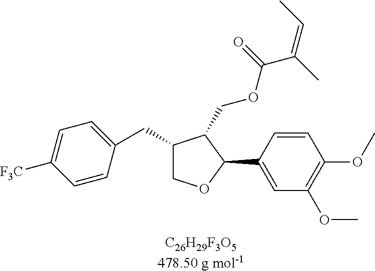 [SMILES: C/C=C(/C)C(=O)OC[C@H]1[C@@H](Cc2ccc(C(F)(F)F)cc2)CO[C@@H]1c1ccc(OC)c(OC)c1 ; "C26H29F3O5478.50 g mol-1"]

Preparation analogous to that of 10-1, using starting material 9-17 (32.7 mg, 0.082 mmol, 1.00 equiv.).

Work-up and purification: Et₂O (5 mL) was added to the reaction content, which was then filtered and rinsed with more Et₂O (15 mL) The solvents were evaporated and the target compound purified by flash column chromatography (9 g silica, flow rate 20 mL/min, gradient 1 to 40% EtOAc in LP within 40 min) to give a colorless oil (34.3 mg, 87%).

[α]_{D}²⁰: +28.5 (c 0.97, MeOH)

HR-MS (ESI) calc'd for M+Na⁺: 501.1859. found: 501.1882.

¹H-NMR (200 MHz, CDCl₃): δ 1.85-1.90 (m, 3H), 2.00 (dq, J=7.2, 1.5 Hz, 3H), 2.55-2.89 (m, 3H), 2.95-3.07 (m, 1H), 3.74 (dd, J=8.7, 5.9 Hz, 1H), 3.87 (s, 3H), 3.88 (s, 3H), 4.07 (dd, J=8.8, 6.0 Hz, 1H), 4.28 (dd, J=11.4, 6.6 Hz, 1H), 4.40 (dd, J=11.4, 7.1 Hz, 1H), 4.84 (d, J=6.4 Hz, 1H), 6.12 (qd, J=7.2, 1.3 Hz, 1H), 6.79-6.92 (m, 3H), 7.30 (d, J=8.1 Hz, 2H), 7.56 (d, J=8.1 Hz, 2H).

### Example 13

### 10-16: (Z)-((2S,3R,4R)-4-(4-butylbenzyl)-2-(3,4-dimethoxyphenyl)tetrahydrofuran-3-yl)methyl 2-methylbut-2-enoate (3026)

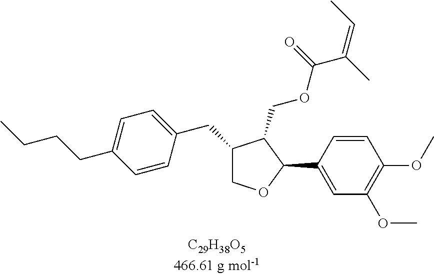 [SMILES: C/C=C(/C)C(=O)OC[C@H]1[C@@H](Cc2ccc(CCCC)cc2)CO[C@@H]1c1ccc(OC)c(OC)c1]

Preparation analogous to that of 10-1, using starting material 9-20 (31.8 mg, 0.083 mmol, 1.00 equiv.) and stirring for 44 in place of 22 h.

Work-up and purification: Et₂O (5 mL) was added to the reaction content, which was then filtered and rinsed with more Et₂O (15 mL) The solvents were evaporated and the target compound purified by flash column chromatography (9 g silica, flow rate 20 mL/min, gradient 1 to 40% EtOAc in LP within 45 min) to give a colorless oil (29.8 mg, 77%).

[α]_{D}²⁵: +17.1 (c 1.54, iPrOH)

¹H-NMR (200 MHz, CDCl₃): δ 0.92 (t, J=7.2 Hz, 3H), 1.35 (sext, J=7.2 Hz, 2H), 1.49-1.67 (m, 2H), 1.84-1.91 (m, 3H), 2.00 (dd, J=7.3, 1.5 Hz, 3H), 2.50-2.86 (m, 5H), 2.91 (dd, J=12.5, 4.2 Hz, 1H), 3.77 (dd, J=8.5, 6.3 Hz, 1H), 3.86 (s, 3H), 3.87 (s, 3H), 4.08 (dd, J=8.6, 6.3 Hz, 1H), 4.28 (dd, J=11.3, 7.0 Hz, 1H), 4.41 (dd, J=11.3, 7.0 Hz, 1H), 4.84 (d, J=6.2 Hz, 1H), 6.09 (qd, J=7.2, 1.4 Hz, 1H), 6.78-6.92 (m, 3H), 7.03-7.15 (m, 4H).

¹³C-NMR (50 MHz, CDCl₃): δ 14.1, 15.9, 20.7, 22.5, 33.3, 33.8, 35.3, 42.6, 49.3, 56.0, 56.1, 62.4, 72.9, 83.0, 109.0, 111.2, 118.2, 127.5, 128.6, 128.7, 135.2, 137.3, 139.0, 141.0, 148.6, 149.2, 167.8.

### Example 14

### 10-18: (Z)-((2S,3R,4R)-4-(4-(1,1-difluoroethyl)benzyl)-2-(3,4-dimethoxyphenyl)tetra-hydrofuran-3-yl)methyl 2-methylbut-2-enoate (3029)

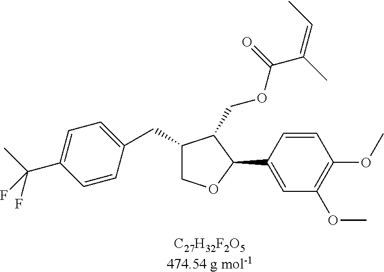 [SMILES: C/C=C(/C)C(=O)OC[C@H]1[C@@H](Cc2ccc(C(C)(F)F)cc2)CO[C@@H]1c1ccc(OC)c(OC)c1 ; "C27H32F2O5474.54 g mol-1"]

Preparation analogous to that of 10-1, using starting material 9-22 (23.2 mg, 0.059 mmol, 1.00 equiv.) and stirring for 44 in place of 22 h.

Work-up and purification: Et₂O (5 mL) was added to the reaction content, which was then filtered and rinsed with more Et₂O (15 mL) The solvents were evaporated and the target compound purified by flash column chromatography (9 g silica, flow rate 20 mL/min, gradient 1 to 40% EtOAc in LP within 40 min) to give a colorless oil (21.8 mg, 78%).

[α]_{D}²⁵: +19.9 (c 1.22, iPrOH)

¹H-NMR (200 MHz, CDCl₃): δ 1.85-1.90 (m, 3H), 1.91 (t, J=18.2 Hz, 3H), 2.00 (dd, J=7.2, 1.5 Hz, 3H), 2.55-2.88 (m, 3H), 2.91-3.03 (m, 1H), 3.75 (dd, J=8.7, 6.0 Hz, 1H), 3.87 (s, 3H), 3.88 (s, 3H), 4.07 (dd, J=8.7, 6.1 Hz, 1H), 4.28 (dd, J=11.3, 6.8 Hz, 1H), 4.41 (dd, J=11.4, 7.1 Hz, 1H), 4.84 (d, J=6.3 Hz, 1H), 6.11 (qd, J=7.2, 1.4 Hz, 1H), 6.80-6.91 (m, 3H), 7.23 (d, J=8.1 Hz, 2H), 7.44 (d, J=8.1 Hz, 2H).

¹³C-NMR (50 MHz, CDCl₃): δ 16.0, 20.7, 26.0 (t, J=29.9 Hz), 33.5, 42.4, 49.3, 56.0, 56.1, 62.2, 72.7, 83.0, 109.0, 111.2, 118.2, 121.9, 125.1 (t, J=5.9 Hz), 127.5, 128.8, 134.9, 139.2, 142.0, 148.7, 149.2, 167.8. One C_{q} not visible.

### Example 15

### 10-19: (Z)-((2S,3R,4R)-2-(4-fluorophenyl)-4-(4-(trifluoromethyl)benzyl)tetrahydro-furan-3-yl)methyl 2-methylbut-2-enoate (3016)

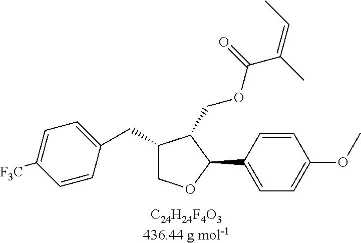

Preparation analogous to that of 10-1, using starting material 9-43 (13.3 mg, 0.038 mmol, 1.0 equiv.) and stirring for 18.5 in place of 22 h.

Work-up and purification: the solvent was evaporated and the target compound purified by flash column chromatography (9 g silica, flow rate 20 mL/min, gradient 2 to 10% EtOAc in LP within 30 min) to give a colorless oil (10.1 mg, 61%).

[α]_{D}²⁰: +13.8 (c 1.02, iPrOH)

HR-MS (APCI-PI) calc'd for M+H⁺: 437.1734. found: 437.1756.

¹H-NMR (200 MHz, CDCl₃): δ 1.83-1.90 (m, 3H), 2.00 (dd, J=7.2, 1.5 Hz, 3H), 2.50-2.96 (m, 3H), 3.00 (d, J=8.7 Hz, 1H), 3.75 (dd, J=8.7, 6.0 Hz, 1H), 4.07 (dd, J=8.7, 6.0 Hz, 1H), 4.27 (dd, J=11.4, 6.9 Hz, 1H), 4.41 (dd, J=11.4, 6.9 Hz, 1H), 4.89 (d, J=6.2 Hz, 1H), 6.12 (qd, J=7.2, 1.4 Hz, 1H), 6.95-7.11 (m, 2H), 7.22-7.36 (m, 4H), 7.56 (d, J=8.2 Hz, 2H).

### Comparative Example 25

### 10-20: (Z)-((2S,3R,4R)-4-(3,4-dimethoxybenzyl)-2-phenyltetrahydrofuran-3-yl)methyl 2-methylbut-2-enoate

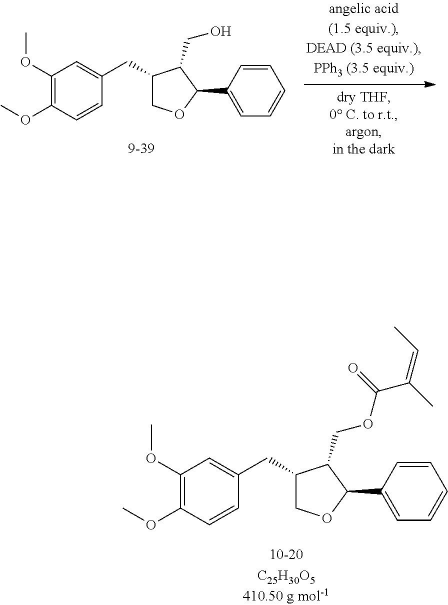 [SMILES: COc1ccc(C[C@H]2CO[C@H](c3ccccc3)[C@H]2CO)cc1OC ; C/C=C(/C)C(=O)OC[C@H]1[C@@H](Cc2ccc(OC)c(OC)c2)CO[C@@H]1c1ccccc1]

Preparation: a reaction vessel was charged with a stir bar, starting material 9-39 (40.4 mg, 0.123 mmol, 1.0 equiv.), angelic acid (18.2 mg, 0.182 mmol, 1.5 equiv.) and PPh₃ (117.5 mg, 0.431 mmol, 3.5 equiv.), and then evacuated and back-filled with argon using standard Schlenk technique (3×). Dry THF (1.5 mL) was then added and the solution cooled to 0° C. in an ice bath. To the stirred mixture was then added DEAD (73 mg, 0.421 mmol, 3.5 equiv.) dropwise via syringe, and the reaction stirred for 18 h while being kept away from light and allowed to warm slowly to room temperature.

Work-up and purification: The solvent was evaporated, which was followed by the addition of CHCl₃ (1.0 mL), LP (10 mL) and water (10 mL). The layers were separated and the aqueous phase reextracted with LP. The solvents were evaporated from the combined organic phases and the target compound first purified by flash column chromatography (90 g silica, flow rate 30 mL/min, isocratically at 5% EtOAc in LP for 5 min, then gradient 5 to 10% within 20 min, then 10 to 30% within 20 min, finally 30 to 100% within 10 min), followed by preparative HPLC (Phenomenex® Luna 10 u C18(2) 100 A, 250×21.20 mm, flow rate 20.0 mL/min, isocratically at 73% MeOH in water for 25 min, then gradient 73 to 77% within 15 min) to give a colorless oil (24.0 mg, 48%).

[α]_{D}²⁰: +17.2 (c 1.13, MeOH)

HR-MS (ESI) calc'd for M+Na⁺: 433.1985. found: 433.1993.

¹H-NMR (200 MHz, CDCl₃): δ 1.84-1.91 (m, 3H), 2.00 (dq, J=7.2, 1.5 Hz, 3H), 2.50-2.85 (m, 3H), 2.89 (dd, J=12.3, 4.2 Hz, 1H), 3.81 (dd, J=8.6, 6.2 Hz, 1H), 3.86 (s, 6H), 4.10 (dd, J=8.6, 6.2 Hz, 1H), 4.28 (dd, J=11.3, 7.1 Hz, 1H), 4.44 (dd, J=11.3, 6.8 Hz, 1H), 4.93 (d, J=5.8 Hz, 1H), 6.10 (qq, J=7.2, 1.4 Hz, 1H), 6.66-6.75 (m, 2H), 6.80 (d, J=7.9 Hz, 1H), 7.21-7.39 (m, 5H).

¹³C-NMR (50 MHz, CDCl₃): δ 16.0, 20.7, 33.2, 42.7, 49.5, 56.0, 56.0, 62.3, 72.9, 83.1, 111.4, 112.0, 120.6, 125.8, 127.5, 127.6, 128.6, 132.7, 139.0, 142.8, 147.6, 149.1, 167.8.

### Comparative Example 26

### 10-21: (Z)-((2S,3R,4R)-4-(3,4-dimethoxybenzyl)-2-(4-fluorophenyl)tetrahydrofuran-3-yl)methyl 2-methylbut-2-enoate (2548)

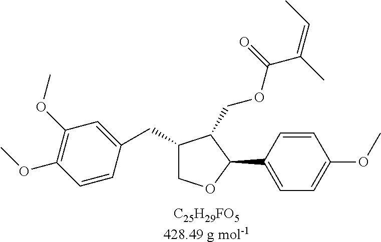

Preparation analogous to that of 10-20, using starting material 9-24 (36.4 mg, 0.105 mmol, 1.0 equiv.).

Work-up and purification: The solvent was evaporated, which was followed by the addition of CHCl₃ (1.0 mL), LP (10 mL) and water (10 mL). The layers were separated and the aqueous phase reextracted with LP. The solvents were evaporated from the combined organic phases and the target compound first purified by flash column chromatography (45 g silica, flow rate 30 mL/min, isocratically at 3% EtOAc in LP for 3 min, then gradient 3 to 50% within 40 min), followed by HPLC (Phenomenex® Luna 10 u C18(2) 100 A, 250×21.20 mm, flow rate 20.0 mL/min, isocratically at 73% MeOH in water for 25 min, then gradient 73 to 77% within 15 min) to give a colorless oil (24.7 mg, 55%).

[α]_{D}²³: +15.9 (c 0.90, MeOH)

HR-MS (ESI) calc'd for M+Na⁺: 451.1891. found: 451.1892.

¹H-NMR (200 MHz, CDCl₃): δ 1.83-1.89 (m, 3H), 2.00 (dq, J=7.2, 1.5 Hz, 3H), 2.49-2.84 (m, 3H), 2.89 (dd, J=12.5, 4.2 Hz, 1H), 3.79 (dd, J=8.6, 6.2 Hz, 1H), 3.86 (s, 3H), 3.86 (s, 3H), 4.08 (dd, J=8.6, 6.2 Hz, 1H), 4.27 (dd, J=11.3, 7.3 Hz, 1H), 4.43 (dd, J=11.3, 6.8 Hz, 1H), 4.89 (d, J=6.1 Hz, 1H), 6.10 (qd, J=7.2, 1.4 Hz, 1H), 6.65-6.84 (m, 3H), 6.95-7.08 (m, 2H), 7.23-7.34 (m, 2H).

### Comparative Example 27

### 10-22: (Z)-((2S,3R,4R)-4-(3,4-dimethoxybenzyl)-2-(3,4-dimethoxyphenyl)tetrahydro-furan-3-yl)methyl 2-methylbut-2-enoate (leoligin) (2418)

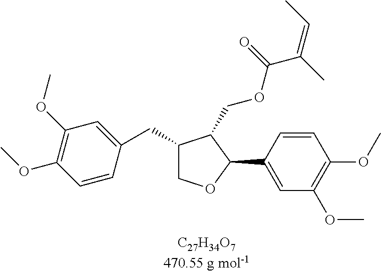

Preparation analogous to that of 10-20, using starting material 9-1 (989 mg, 2.55 mmol, 1.0 equiv.), THF (20 mL) for the reaction and stirring for 12 h.

Work-up and purification: The solvent was evaporated, which was followed by the addition of CHCl₃ (15 mL), LP (300 mL) and water (200 mL). The layers were separated and the aqueous phase reextracted with LP (4×50 ml). The solvents were evaporated from the combined organic phases and the target compound purified by flash column chromatography (180 g silica, flow rate 40 mL/min, gradient 25 to 50% EtOAc in LP within 60 min) to give an almost colorless (1.124 g, 94%).

[α]_{D}²⁵: +23.4 (c 3.69, MeOH)

HR-MS (ESI) calc'd for M+Na⁺: 493.2197. found: 493.2201.

¹H-NMR (200 MHz, CDCl₃): δ 1.85-1.90 (m, 3H), 2.00 (dq, J=7.2, 1.5 Hz, 3H), 2.49-2.82 (m, 3H), 2.90 (dd, J=12.4, 4.2 Hz, 1H), 3.78 (dd, J=8.6, 6.0 Hz, 1H), 3.86 (s, 3H), 3.87 (s, 6H), 3.88 (s, 3H), 4.08 (dd, J=8.6, 6.2 Hz, 1H), 4.28 (dd, J=11.3, 7.0 Hz, 1H), 4.42 (dd, J=11.3, 7.0 Hz, 1H), 4.84 (d, J=6.3 Hz, 1H), 6.10 (qd, J=7.2, 1.3 Hz, 1H), 6.67-6.75 (m, 2H), 6.77-6.90 (m, 4H).

¹³C-NMR (50 MHz, CDCl₃): δ 15.9, 20.7, 33.4, 42.8, 49.4, 56.0, 56.0, 56.1, 56.1, 62.3, 72.9, 83.0, 109.2, 111.3, 111.6, 112.2, 118.2, 120.6, 127.6, 132.8, 135.2, 138.9, 147.8, 148.7, 149.2, 149.3, 167.8.

### Comparative Example 28

### 10-23: (Z)-((2S,3R,4R)-4-(3,4-dimethoxybenzyl)-2-(4-methoxyphenyl)tetrahydrofuran-3-yl)methyl 2-methylbut-2-enoate (3030)

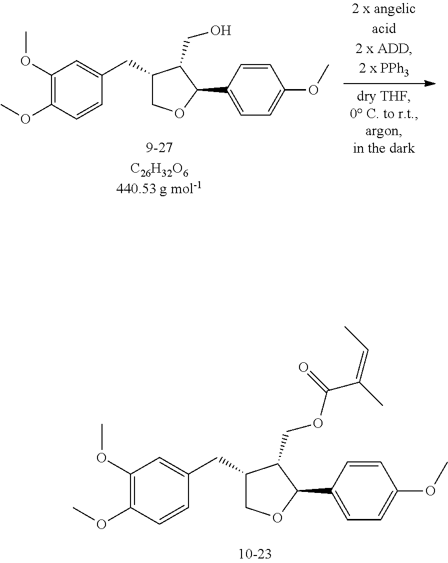

Preparation: a reaction vessel was charged with a stir bar, starting material 9-27 (20.6 mg, 0.057 mmol, 1.0 equiv.), angelic acid (8.6 mg, 0.086 mmol, 1.5 equiv.) and PPh₃ (52.7 mg, 0.201 mmol, 3.5 equiv.), and then evacuated and back-filled with argon using standard Schlenk technique (3×). Dry THF (0.75 mL) was then added and the solution cooled to 0° C. in an ice bath. To the stirred mixture was then added a solution of ADD (50.7 mg, 0.201 mmol, 3.5 equiv.) in dry THF (1.0 mL) via syringe over approximately 1 min, and the reaction stirred for 46 h while being kept away from light and allowed to warm slowly to room temperature (first leg). Then the reaction was cooled in an ice bath again, and there was added more angelic acid (4.3 mg, 0.043 mmol, 0.8 equiv.) and PPh₃ (26.3 mg, 0.100 mmol, 1.8 equiv.) in dry THF (0.75 mL) via syringe, followed by the addition of more ADD (25.3 mg, 0.100 mmol, 1.8 equiv.) in dry THF (1.0 ml) via syringe over approximately 1 min, and the reaction stirred for 24 h while being kept away from light and allowed to warm slowly to room temperature again (second leg).

Work-up and purification: Et₂O (5 mL) was added to the reaction content, which was then filtered and rinsed with more Et₂O (15 mL) The solvents were evaporated and the target compound purified by flash column chromatography (9 g silica, flow rate 20 mL/min, gradient 1 to 10% EtOAc in heptane within 10 min, then 10 to 70% within 50 min) to give a colorless oil (22.6 mg, 89%).

[α]_{D}²⁵: +13.5 (c 1.22, iPrOH)

¹H-NMR (200 MHz, CDCl₃): δ 1.84-1.90 (m, 3H), 2.00 (dd, J=7.2, 1.5 Hz, 3H), 2.48-2.84 (m, 3H), 2.89 (dd, J=12.6, 4.1 Hz, 1H), 3.72-3.82 (m, 1H), 3.80 (s, 3H), 3.86 (s, 3H), 3.86 (s, 3H), 4.07 (dd, J=8.6, 6.1 Hz, 1H), 4.26 (dd, J=11.3, 6.9 Hz, 1H), 4.41 (dd, J=11.3, 7.1 Hz, 1H), 4.85 (d, J=6.3 Hz, 1H), 6.08 (qd, J=7.2, 1.4 Hz, 1H), 6.65-6.92 (m, 5H), 7.24 (d, J=7.9 Hz, 2H).

¹³C-NMR (50 MHz, CDCl₃): δ 16.0, 20.7, 33.3, 42.8, 49.4, 55.4, 56.0, 56.0, 62.3, 72.8, 82.8, 111.5, 112.0, 114.0, 120.6, 127.1, 127.6, 132.8, 134.7, 138.9, 147.6, 149.1, 159.2, 167.8.

### Comparative Example 29

### 10-24: (Z)-((2S,3R,4R)-4-(4-methoxybenzyl)-2-phenyltetrahydrofuran-3-yl)methyl 2-methylbut-2-enoate (2859)

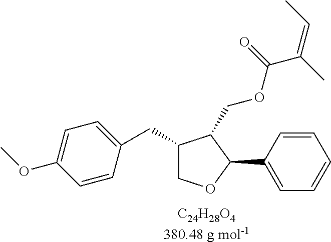

Preparation analogous to that of 10-23, using starting material 9-40 (10.1 mg, 0.034 mmol, 1.0 equiv.). First leg: angelic acid (5.1 mg, 0.051 mmol, 1.5 equiv.), ADD (30.0 mg, 0.119 mmol, 3.5 equiv.), PPh₃ (31.1 mg, 0.119 mmol, 3.5 equiv.), stirring for 16 h. Second leg: angelic acid (5.1 mg, 0.051 mmol, 1.5 equiv.), ADD (30.0 mg, 0.119 mmol, 3.5 equiv.), PPh₃ (31.1 mg, 0.119 mmol, 3.5 equiv.), stirring for 16 h.

Work-up and purification: the solvent was evaporated and the target compound purified by flash column chromatography (18 g silica, flow rate 20 mL/min, gradient 1 to 35% EtOAc in LP within 35 min) to give a colorless oil (3.7 mg, 29%).

¹H-NMR (200 MHz, CDCl₃): δ 1.84-1.91 (m, 3H), 2.00 (dd, J=7.3, 1.5 Hz, 3H), 2.50-2.81 (m, 3H), 2.88 (dd, J=12.3, 4.1 Hz, 1H), 3.74-3.83 (m, 4H), 4.09 (dd, J=8.5, 6.2 Hz, 1H), 4.28 (dd, J=11.3, 7.2 Hz, 1H), 4.43 (dd, J=11.3, 6.8 Hz, 1H), 4.92 (d, J=5.8 Hz, 1H), 6.10 (qd, J=7.2, 1.4 Hz, 1H), 6.79-6.88 (m, 2H), 7.04-7.13 (m, 2H), 7.22-7.37 (m, 5H).

### Example 16

### 10-25: (Z)-((2S,3R,4R)-2-phenyl-4-(4-(trifluoromethyl)benzyl)tetrahydrofuran-3-yl)-methyl 2-methylbut-2-enoate (301.5)

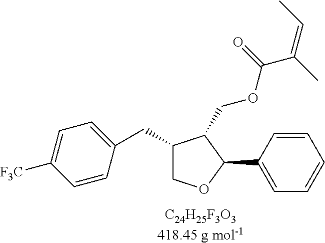

Preparation analogous to that of 10-23, using starting material 9-41 (15.3 mg, 0.045 mmol, 1.0 equiv.). First leg: angelic acid (6.8 mg, 0.068 mmol, 1.5 equiv.), ADD (40.1 mg, 0.159 mmol, 3.5 equiv.), PPh₃ (41.7 mg, 0.159 mmol, 3.5 equiv.), stirring for 16 h. Second leg: angelic acid (6.8 mg, 0.068 mmol, 1.5 equiv.), ADD (40.1 mg, 0.159 mmol, 3.5 equiv.), PPh₃ (41.7 mg, 0.159 mmol, 3.5 equiv.), stirring for 16 h.

Work-up and purification: the solvent was evaporated and the target compound purified by flash column chromatography (18 g silica, flow rate 20 mL/min, gradient 2 to 10% EtOAc in LP within 30 min) to give a colorless oil (13.6 mg, 72%).

[α]_{D}²⁰: +16.3 (c 0.74, iPrOH)

HR-MS (APCI-PI) calc'd for M+H+: 419.1829. found: 419.1840.

¹H-NMR (200 MHz, CDCl₃): δ 1.85-1.91 (m, 3H), 2.00 (dq, J=7.2, 1.5 Hz, 3H), 2.56-2.91 (m, 3H), 3.00 (d, J=8.6 Hz, 1H), 3.77 (dd, J=8.7, 6.1 Hz, 1H), 4.09 (dd, J=8.7, 6.0 Hz, 1H), 4.29 (dd, J=11.3, 6.8 Hz, 1H), 4.42 (dd, J=11.4, 7.0 Hz, 1H), 4.93 (d, J=5.9 Hz, 1H), 6.11 (qd, J=7.2, 1.3 Hz, 1H), 7.22-7.40 (m, 7H), 7.55 (d, J=8.2 Hz, 2H).

### Comparative Example 30

### 10-26: (Z)-((2S,3R,4R)-2-(4-fluorophenyl)-4-(4-methoxybenzyl)tetrahydrofuran-3-yl)-methyl 2-methylbut-2-enoate (3011)

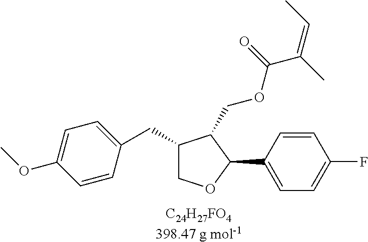

Preparation analogous to that of 10-23, using starting material 9-25 (27.9 mg, 0.088 mmol, 1.0 equiv.). First leg: angelic acid (13.2 mg, 0.132 mmol, 1.5 equiv.), ADD (77.9 mg, 0.309 mmol, 3.5 equiv.), PPh₃ (81.0 mg, 0.309 mmol, 3.5 equiv.), stirring for 16 h. Second leg: angelic acid (13.2 mg, 0.132 mmol, 1.5 equiv.), ADD (77.9 mg, 0.309 mmol, 3.5 equiv.), PPh₃ (81.0 mg, 0.309 mmol, 3.5 equiv.), stirring for 21 h.

Work-up and purification: the solvent was evaporated and the target compound purified by two sequential runs of flash column chromatography (first run: 18 g silica, flow rate 20 mL/min, gradient 1 to 35% EtOAc in LP within 35 min; second run: 18 g silica, flow rate 20 mL/min, gradient 1 to 20% EtOAc in LP within 35 min) to give a colorless oil (16.7 mg, 48%).

[α]_{D}²⁰: +9.2 (c 0.75, iPrOH)

HR-MS (APCI-PI) calc'd for M+H+: 399.1966. found: 399.1972.

¹H-NMR (200 MHz, CDCl₃): δ 1.83-1.90 (m, 3H), 1.96-2.03 (m, 3H), 2.49-2.93 (m, 4H), 3.72-3.83 (m, 4H), 4.07 (dd, J=8.6, 6.2 Hz, 1H), 4.26 (dd, J=11.3, 7.3 Hz, 1H), 4.41 (dd, J=11.3, 6.7 Hz, 1H), 4.88 (d, J=6.0 Hz, 1H), 6.10 (qd, J=7.2, 1.4 Hz, 1H), 6.79-6.88 (m, 2H), 6.95-7.13 (m, 4H), 7.22-7.34 (m, 2H).

### Example 17

### 10-27: (Z)-((2S,3R,4R)-4-(4-fluorobenzyl)-2-(4-fluorophenyl)tetrahydrofuran-3-yl)-methyl 2-methylbut-2-enoate (2864)

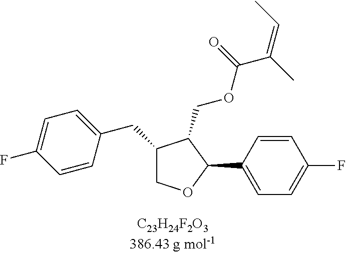 [SMILES: C/C=C(/C)C(=O)OC[C@H]1[C@@H](Cc2ccc(F)cc2)CO[C@@H]1c1ccc(F)cc1]

Preparation analogous to that of 10-23, using starting material 9-26 (20.9 mg, 0.069 mmol, 1.0 equiv.). First leg: angelic acid (10.3 mg, 0.103 mmol, 1.5 equiv.), ADD (60.7 mg, 0.241 mmol, 3.5 equiv.), PPh₃ (63.1 mg, 0.241 mmol, 3.5 equiv.), stirring for 16 h. Second leg: angelic acid (10.3 mg, 0.103 mmol, 1.5 equiv.), ADD (60.7 mg, 0.241 mmol, 3.5 equiv.), PPh₃ (63.1 mg, 0.241 mmol, 3.5 equiv.), stirring for 21 h.

Work-up and purification: the solvent was evaporated and the target compound purified by flash column chromatography (18 g silica, flow rate 20 mL/min, gradient 0 to 10% EtOAc in LP within 50 min) to give a colorless oil (17.1 mg, 64%).

[α]_{D}²⁵: +15.2 (c 1.13, iPrOH)

HR-MS (APCI-PI) calc'd for M+H⁺: 387.1766. found: 387.1773.

¹H-NMR (200 MHz, CDCl₃): δ 1.83-1.90 (m, 3H), 1.99 (dd, J=7.3, 1.5 Hz, 3H), 2.49-2.83 (m, 3H), 2.91 (dd, J=12.2, 3.7 Hz, 1H), 3.75 (dd, J=8.7, 6.2 Hz, 1H), 4.06 (dd, J=8.7, 6.1 Hz, 1H), 4.26 (dd, J=11.4, 7.1 Hz, 1H), 4.40 (dd, J=11.4, 6.8 Hz, 1H), 4.88 (d, J=6.1 Hz, 1H), 6.11 (qq, J=7.3, 1.4 Hz, 1H), 6.92-7.18 (m, 6H), 7.23-7.34 (m, 2H).

### Example 18

### 10-28: (Z)-((2S,3R,4R)-4-(4-(tert-butyl)benzyl)-2-(4-fluorophenyl)tetrahydrofuran-3-yl)methyl 2-methylbut-2-enoate (3010)

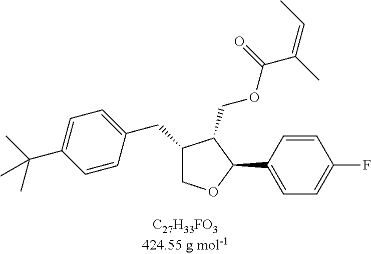 [SMILES: C/C=C(/C)C(=O)OC[C@H]1[C@@H](Cc2ccc(C(C)(C)C)cc2)CO[C@@H]1c1ccc(F)cc1]

Preparation analogous to that of 10-23, using starting material 9-42 (10.5 mg, 0.031 mmol, 1.0 equiv.). First leg: angelic acid (4.6 mg, 0.046 mmol, 1.5 equiv.), ADD (27.1 mg, 0.108 mmol, 3.5 equiv.), PPh₃ (28.2 mg, 0.108 mmol, 3.5 equiv.), stirring for 18.5 h. Second leg: angelic acid (4.6 mg, 0.046 mmol, 1.5 equiv.), ADD (27.1 mg, 0.108 mmol, 3.5 equiv.), PPh₃ (28.2 mg, 0.108 mmol, 3.5 equiv.), stirring for 47 h.

Work-up and purification: the solvent was evaporated and the target compound purified by flash column chromatography (9 g silica, flow rate 20 mL/min, gradient 2 to 10% EtOAc in LP within 40 min) to give a colorless oil (4.9 mg, 37%).

[α]_{D}²⁰: +9.2 (c 0.69, iPrOH)

HR-MS (APCI-PI) calc'd for M+H+: 425.2486. found: 425.2503.

¹H-NMR (200 MHz, CDCl₃): δ 1.83-1.90 (m, 3H), 1.31 (s, 9H), 1.99 (dd, J=7.2, 1.5 Hz, 3H), 2.49-2.85 (m, 3H), 2.90 (dd, J=12.5, 4.2 Hz, 1H), 3.78 (dd, J=8.6, 6.4 Hz, 1H), 4.09 (dd, J=8.6, 6.3 Hz, 1H), 4.28 (dd, J=11.3, 7.3 Hz, 1H), 4.42 (dd, J=11.3, 6.7 Hz, 1H), 4.89 (d, J=5.9 Hz, 1H), 5.98-6.21 (m, 1H), 6.95-7.15 (m, 4H), 7.22-7.37 (m, 4H).

### Example 19

### 10-29: ((2S,3R,4R)-4-(3,4-dimethoxybenzyl)-2-(3,4,5-trimethoxyphenyl)tetrahydro-furan-3-yl)methyl 3-methylbut-2-enoate (2755)

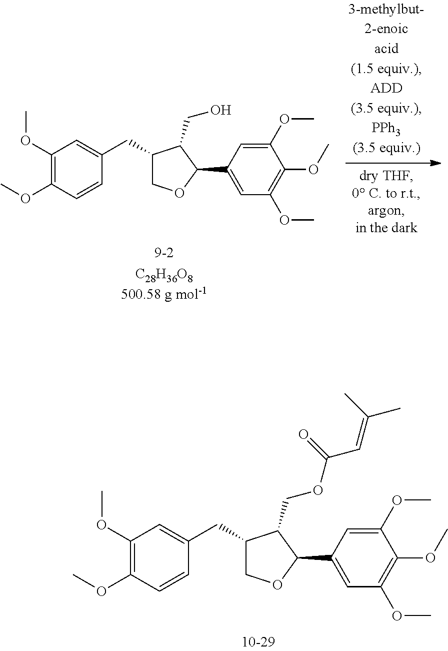

Preparation: a reaction vessel was charged with a stir bar, starting material 9-2 (21.2 mg, 0.051 mmol, 1.0 equiv.), 3-methylbut-2-enoic acid (7.7 mg, 0.077 mmol, 1.5 equiv.) and PPh₃ (46.8 mg, 0.179 mmol, 3.5 equiv.), and then evacuated and back-filled with argon using standard Schlenk technique (3×). Dry THF was then added to achieve a 0.13 M solution which was cooled to 0° C. in an ice bath. To the stirred mixture was then added a solution of ADD (45.2 mg, 0.179 mmol, 3.5 equiv.) in dry THF via syringe over approximately 1 min, and the reaction stirred for 18.5 h while being kept away from light and allowed to warm slowly to room temperature.

Work-up and purification: To the reaction mixture was added saturated brine, the layers separated and the aqueous phase extracted with Et₂O (3×). The combined organic phases were dried over Na₂SO₄, filtered and the solvents evaporated. The target compound purified by flash column chromatography (9 g silica, flow rate 20 mL/min, gradient 10 to 22% EtOAc in LP within 10 min, then isocratically at 22% for 6 min, then 22 to 65% within 30 min) to give a slightly yellow, viscous oil (16.6 mg, 66%).

[α]_{D}²⁰: +29.9 (c 1.19, MeOH)

HR-MS (ESI) calc'd for M+Na⁺: 523.2302. found: 523.2310.

¹H-NMR (200 MHz, CDCl₃): δ 1.89 (d, J=1.0 Hz, 3H), 2.18 (d, J=1.2 Hz, 3H), 2.46-2.94 (m, 3H), 3.87 (dd, J=12.6, 4.2 Hz, 1H), 3.74 (dd, J=8.6, 6.7 Hz, 1H), 3.81 (s, 3H), 3.84 (s, 9H), 3.86 (s, 3H), 4.06 (dd, J=8.6, 6.3 Hz, 1H), 4.22 (dd, J=11.3, 7.2 Hz, 1H), 4.39 (dd, J=11.3, 6.7 Hz, 1H), 4.79 (d, J=5.9 Hz, 1H), 5.65 (m, 1H), 6.54 (s, 2H), 6.65-6.84 (m, 3H).

¹³C-NMR (50 MHz, CDCl₃): δ 20.3, 27.5, 33.1, 42.5, 49.1, 55.9, 56.1, 60.8, 61.7, 72.8, 83.2, 102.5, 111.3, 111.9, 115.5, 120.4, 132.6, 138.3, 147.4, 148.9, 153.2, 157.7, 166.4. One C_{q} not visible.

### Comparative Example 31

### 10-30: (Z)-((2S,3R,4R)-4-benzyl-2-(3,4,5-trimethoxyphenyl)tetrahydrofuran-3-yl)-methyl 2-methylbut-2-enoate (2764)

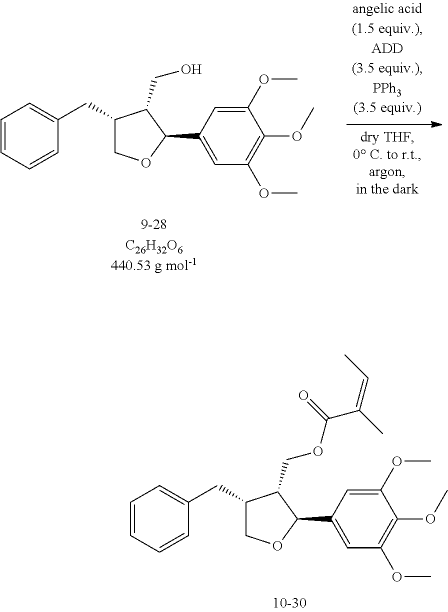 [SMILES: COc1cc([C@H]2OC[C@H](Cc3ccccc3)[C@@H]2CO)cc(OC)c1OC ; C/C=C(/C)C(=O)OC[C@H]1[C@@H](Cc2ccccc2)CO[C@@H]1c1cc(OC)c(OC)c(OC)c1]

Preparation: a reaction vessel was charged with a stir bar, starting material 9-28 (16.1 mg, 0.045 mmol, 1.0 equiv.), angelic acid (6.8 mg, 0.068 mmol, 1.5 equiv.) and PPh₃ (41.3 mg, 0.158 mmol, 3.5 equiv.), and then evacuated and back-filled with argon using standard Schlenk technique (3×). Dry THF was then added to achieve a 0.13 M solution which was cooled to 0° C. in an ice bath. To the stirred mixture was then added a solution of ADD (39.9 mg, 0.158 mmol, 3.5 equiv.) in dry THF via syringe over approximately 1 min, and the reaction stirred for 18.5 h while being kept away from light and allowed to warm slowly to room temperature.

Work-up and purification: To the reaction mixture was added saturated brine, the layers separated and the aqueous phase extracted with Et₂O (3×). The combined organic phases were dried over Na₂SO₄, filtered and the solvents evaporated. The target compound purified by flash column chromatography (9 g silica, flow rate 20 mL/min, gradient 10 to 22% EtOAc in LP within 10 min, then isocratically at 22% for 6 min, then 22 to 65% within 30 min) to give a slightly yellow, viscous oil (12.1 mg, 61%).

[α]_{D}²⁰: +28.1 (c 0.70, MeOH)

¹H-NMR (400 MHz, CDCl₃): δ 1.85-1.89 (m, 3H), 1.97-2.02 (m, 3H), 2.58-2.67 (m, 2H), 2.72-2.83 (m, 1H), 2.93 (dd, J=13.3, 4.8 Hz, 1H), 3.77 (dd, J=8.5, 7.0 Hz, 1H), 3.82 (s, 3H), 3.84 (s, 6H), 4.07 (dd, J=8.5, 6.7 Hz, 1H), 4.30 (dd, J=11.4, 7.3, 1H), 4.42 (dd, J=11.4, 7.0 Hz, 1H), 4.83 (d, J=6.1, 1H), 6.06-6.14 (m, 1H), 6.53 (s, 2H), 7.15-7.32 (m, 5H).

¹³C-NMR (100 MHz, CDCl₃): δ 15.8, 20.6, 33.5, 42.3, 49.2, 56.1, 60.8, 62.2, 72.8, 83.2, 102.5, 126.3, 127.3, 128.6, 128.6, 137.2, 138.2, 139.1, 134.0, 153.3, 167.7.

### Example 20

### 10-31: (Z)-((2S,3R,4R)-4-(4-(tert-butyl)benzyl)-2-(3,4,5-trimethoxyphenyl)tetrahydro-furan-3-yl)methyl 2-methylbut-2-enoate (2765)

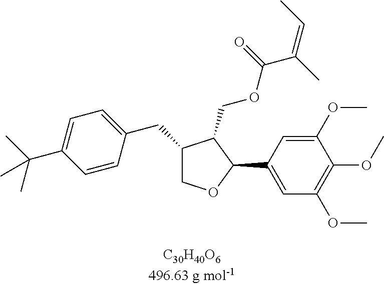 [SMILES: C/C=C(/C)C(=O)OC[C@H]1[C@@H](Cc2ccc(C(C)(C)C)cc2)CO[C@@H]1c1cc(OC)c(OC)c(OC)c1]

Preparation analogous to, work-up and purification identical to that of 10-30, using starting material 9-29 (23.7 mg, 0.057 mmol, 1.0 equiv.) to give a slightly yellow, viscous oil (19.1 mg, 76%).

[α]_{D}²⁰: +22.4 (c 0.1.49, MeOH)

HR-MS (ESI) calc'd for M+Na⁺: 519.2717. found: 519.2717.

¹H-NMR (200 MHz, CDCl₃): δ 1.30 (s, 9H), 1.82-1.92 (m, 3H), 1.94-2.05 (m, 3H), 2.51-2.97 (m, 4H), 3.81 (s, 4H), 3.83 (s, 6H), 4.09 (dd, J=8.6, 6.3 Hz, 1H), 4.29 (dd, J=11.4, 7.2 Hz, 1H), 4.43 (dd, J=11.4, 6.8 Hz, 1H), 4.83 (d, J=5.9 Hz, 1H), 6.02-6.17 (m, 1H), 6.54 (s, 2H), 7.05-7.14 (m, 2H), 7.27-7.36 (m, 2H).

### Comparative Example 32

### 10-33: (Z)-((2S,3R,4R)-4-(4-(trifluoromethyl)benzyl)-2-(3,4,5-trimethoxyphenyl)-tetrahydrofuran-3-yl)methyl 2-methylbut-2-enoate (2769)

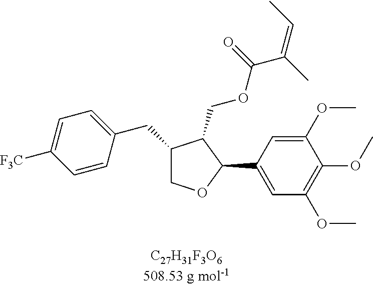 [SMILES: C/C=C(/C)C(=O)OC[C@H]1[C@@H](Cc2ccc(C(F)(F)F)cc2)CO[C@@H]1c1cc(OC)c(OC)c(OC)c1]

Preparation analogous to, work-up and purification identical to that of 10-30, using starting material 9-32 (35.2 mg, 0.083 mmol, 1.0 equiv.) to give a slightly yellow, viscous oil (31.2 mg, 74%).

[α]_{D}²⁰: +26.2 (c 1.30, MeOH)

HR-MS (ESI) calc'd for M+Na⁺: 531.1965. found: 531.1973.

¹H-NMR (200 MHz, CDCl₃): δ 1.85-1.88 (m, 3H), 1.97-2.02 (m, 3H), 2.59-2.83 (m, 3H), 2.99 (dd, J=12.9, 4.1 Hz, 1H), 3.74 (dd, J=8.6, 6.9 Hz, 1H), 3.82 (s, 3H), 3.84 (s, 6H), 4.06 (dd, J=8.6, 6.39 Hz, 1H), 4.30 (dd, J=11.4, 7.0 Hz, 1H), 4.41 (dd, J=11.4, 7.0 Hz, 1H), 4.83 (d, J=6.1 Hz, 1H), 6.07-6.15 (m, 1H), 6.53 (s, 2H), 7.29 (d, J=8.1 Hz, 2H), 7.55 (d, J=8.1 Hz, 2H).

### Comparative Example 33

### 10-34: (Z)-((2S,3R,4R)-4-(4-methoxybenzyl)-2-(3,4,5-trimethoxyphenyl)tetrahydro-furan-3-yl)methyl 2-methylbut-2-enoate (2770)

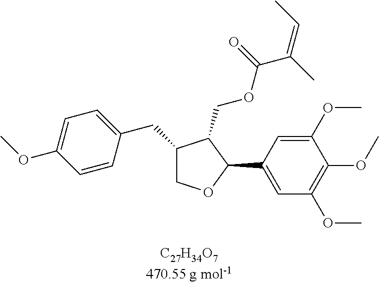

Preparation analogous to, work-up and purification identical to that of 10-30, using starting material 9-30 (14.3 mg, 0.037 mmol, 1.0 equiv.) to give a slightly yellow, viscous oil (10.1 mg, 58%).

[α]_{D}²⁰: +20.1 (c 1.00, MeOH)

¹H-NMR (200 MHz, CDCl₃): δ 1.82-1.91 (m, 3H), 1.94-2.04 (m, 3H), 2.48-2.80 (m, 3H), 2.87 (dd, J=12.4, 4.2 Hz, 1H), 3.78 (s, 4H), 3.81 (s, 3H), 3.84 (s, 6H), 4.07 (dd, J=8.6, 6.3 Hz, 1H), 4.28 (dd, J=11.4, 7.2 Hz, 1H), 4.42 (dd, J=11.4, 6.7, 1H), 4.83 (d, J=5.7, 1H), 6.10 (m, 1H), 6.53 (s, 2H), 6.8-6.9 (m, 2H), 7.03-7.13 (m, 2H).

¹³C-NMR (50 MHz, CDCl₃): δ 15.8, 20.6, 32.6, 42.5, 49.1, 55.2, 56.1, 60.8, 62.2, 72.8, 83.2, 102.5, 114.0, 127.3, 129.5, 131.9, 138.3, 139.1, 153.3, 158.1, 167.7. One C_{q} not visible.

### Example 21

### 10-35: (Z)-((2S,3R,4R)-4-(4-acetylbenzyl)-2-(3,4,5-trimethoxyphenyl)tetrahydrofuran-3-yl)methyl 2-methylbut-2-enoate (2771)

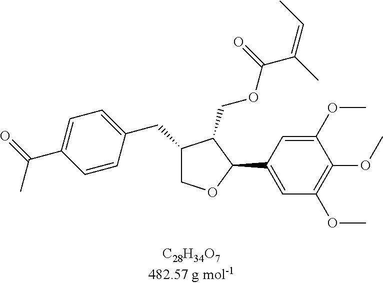 [SMILES: C/C=C(/C)C(=O)OC[C@H]1[C@@H](Cc2ccc(C(C)=O)cc2)CO[C@@H]1c1cc(OC)c(OC)c(OC)c1]

Preparation analogous to, work-up and purification identical to that of 10-30, using starting material 9-36 (14.3 mg, 0.036 mmol, 1.0 equiv.) to give a slightly yellow, viscous oil (13.1 mg, 76%).

[α]_{D}²⁰: +19.7 (c 1.28, MeOH)

¹H-NMR (200 MHz, CDCl₃): δ 1.82-1.92 (m, 3H), 1.94-2.05 (m, 3H), 2.58 (s, 3H), 2.61-2.91 (m, 3H), 2.92-3.05 (m, 1H), 3.74 (dd, J=8.8, 6.2 Hz, 1H), 3.81 (s, 3H), 3.84 (s, 6H), 4.06 (dd, J=8.8, 6.1 Hz, 1H), 4.29 (dd, J=11.4 & 7.0, 1 H), 4.41 (dd, J=11.4, 6.9 Hz, 1H), 4.83 (d, J=6.0, 1 H), 6.03-6.19 (m, 1H), 6.52 (s, 2H), 7.27 (d, J=8.2 Hz, 2H), 7.89 (d, J=8.2 Hz, 2H).

¹³C-NMR (50 MHz, CDCl₃): δ 15.8, 20.6, 26.5, 33.6, 42.8, 49.2, 56.1, 60.8, 62.0, 72.6, 83.1, 102.5, 128.8, 128.8, 127.2, 135.5, 136.9, 137.3, 137.9, 139.3, 153.3, 167.6, 197.6.

### Example 22

### 10-38: ethyl 4-(((3R,4R,5S)-4-((((Z)-2-methylbut-2-enoyl)oxy)methyl)-5-(3,4,5-trimethoxyphenyl)tetrahydrofuran-3-yl)methyl)benzoate (2774)

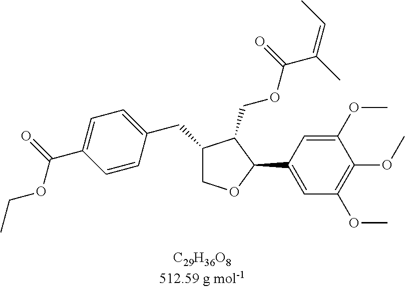 [SMILES: C/C=C(/C)C(=O)OC[C@H]1[C@@H](Cc2ccc(C(=O)OCC)cc2)CO[C@@H]1c1cc(OC)c(OC)c(OC)c1]

Preparation analogous to, work-up and purification identical to that of 10-30, using starting material 9-37 (34.0 mg, 0.079 mmol, 1.0 equiv.) to give a slightly yellow, viscous oil (29.8 mg, 74%).

[α]_{D}²⁰: +17.7 (c 1.23, MeOH)

¹H-NMR (400 MHz, CDCl₃): δ 1.39 (t, J=7.16 Hz, 3H), 1.85-1.91 (m, 3H), 1.98-2.04 (m, 3H), 2.59-2.85 (m, 3H), 3.00 (dd, J=13.0 & 4.5 Hz, 1H), 3.75 (dd, J=8.5, 6.7 Hz, 1H), 3.83 (s, 3H), 3.85 (s, 6H), 4.06 (dd, J=8.5 & 6.6 Hz, 1H), 4.27-4.46 (m, 4H), 4.84 (d, J=6.14, 1H), 6.08-6.16 (m, 1H), 6.54 (s, 2H), 7.25 (d, J=8.5 Hz, 2H), 7.99 (d, J=8.5 Hz, 2H).

¹³C-NMR (50 MHz, CDCl₃): δ 14.3, 15.8, 20.5, 33.6, 42.1, 49.2, 56.0, 60.8, 60.9, 62.0, 72.6, 83.0, 102.5, 127.2, 128.5, 128.7, 129.9, 137.2, 138.0, 139.2, 142.3, 153.3, 166.4, 167.6.

### Comparative Example 34

### 10-39: (Z)-((2S,3R,4R)-4-(4-methylbenzyl)-2-(3,4,5-trimethoxyphenyl)tetrahydrofuran-3-yl)methyl 2-methylbut-2-enoate (2775)

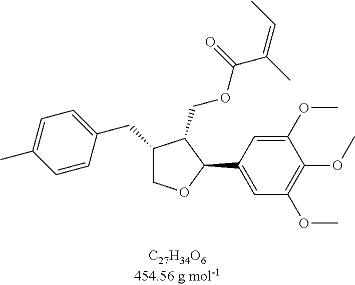

Preparation analogous to, work-up and purification identical to that of 10-30, using starting material 9-33 (23.7 mg, 0.064 mmol, 1.0 equiv.) to give a slightly yellow, viscous oil (18.6 mg, 64%).

[α]_{D}²⁰: +25.3 (c 1.40, MeOH)

¹H-NMR (200 MHz, CDCl₃): δ 1.84-1.92 (m, 3H), 1.96-2.05 (m, 3H), 2.32 (s, 3H), 2.51-2.83 (m, 3H), 2.90 (dd, J=12.3, 4.1 Hz, 1H), 3.77 (dd, J=8.6, 6.5 Hz, 1H), 3.82 (s, 3H), 3.85 (s, 6H), 4.08 (dd, J=8.6, 6.4 Hz, 1H), 4.30 (dd, J=11.4, 7.2, 1 H), 4.43 (dd, J=11.4, 6.7 Hz, 1H), 4.84 (d, J=5.9, 1H), 6.03-6.19 (m, 1H), 6.54 (s, 2H), 7.00-7.18 (m, 4H).

¹³C-NMR (50 MHz, CDCl₃): δ 15.8, 20.6, 20.9, 33.1, 42.4, 49.2, 56.1, 60.8, 62.3, 72.8, 83.18, 102.5, 127.3, 128.5, 129.3, 135.9, 136.9, 138.3, 139.0, 153.3, 167.7. One C_{q} not visible.

### Examples 23 to 41 and Comparative Examples 35 to 46

Within this group of examples, free alcohols of formula (9) are transformed into compounds of formula (10) through Steglich esterification.

### Comparative Example 35

### 10-41: (E)-((2S,3R,4R)-4-(3,4-dimethoxybenzyl)-2-(3,4-dimethoxyphenyl)tetrahydro-furan-3-yl)methyl 2-methylbut-2-enoate (2539)

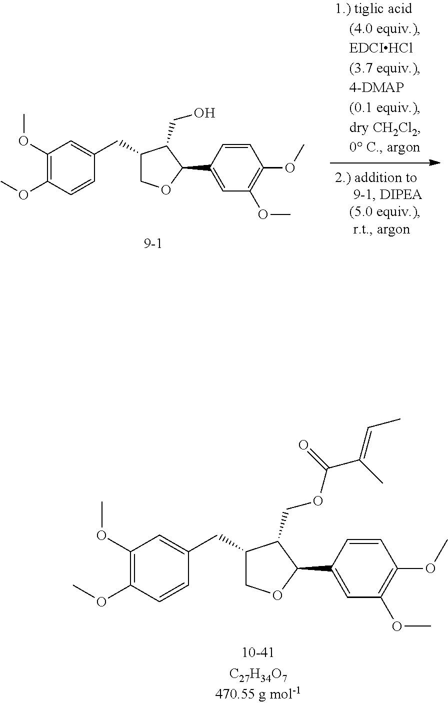

Preparation: a reaction vessel was charged with a stir bar, tiglic acid (36.0 mg, 0.360 mmol, 4.0 equiv.) and 4-DMAP (1.1 mg, 9.0 μmol, 0.1 equiv.), and then evacuated and back-filled with argon using standard Schlenk technique (1×). Then was added dry CH₂Cl₂ (1.0 mL) via syringe and the solution cooled to 0° C. in an ice bath. The vessel was briefly opened, EDCI.HCl (63.8 mg, 0.333 mmol, 3.7 equiv.) added in one go and the mixture stirred for 3 h at 0° C. Meanwhile, a second vessel was charged with a stir bar and 9-1 (35.0 mg, 0.090 mmol, 1.00 equiv.), evacuated and back-filled with argon (3×), and DIPEA (78 μL, 0.45 mmol, 5.0 equiv.) was added via syringe. After 3 h, the solution containing the activated carboxylic acid was transferred to the second vial via syringe and stirred for 16 h at room temperature.

Work-up and purification: the reaction solution was used directly for flash column chromatography (9 g silica, flow rate 20 mL/min, gradient 10 to 50% EtOAc in LP within 30 min) to give a nearly colorless oil (40.3 mg, 95%).

[α]_{D}²⁰: +17.5 (c 2.48, MeOH)

HR-MS (ESI) calc'd for M+Na⁺: 493.2197. found: 493.2204.

¹H-NMR (200 MHz, CDCl₃): δ 1.73-1.84 (m, 6H), 2.48-2.97 (m, 4H), 3.77 (dd, J=8.6, 6.2 Hz, 1H), 3.86 (s, 3H), 3.87 (s, 6H), 3.88 (s, 3H), 4.09 (dd, J=8.6, 6.2 Hz, 1H), 4.26 (dd, J=11.3, 7.0 Hz, 1H), 4.43 (dd, J=11.3, 6.6 Hz, 1H), 4.83 (d, J=6.3 Hz, 1H), 6.67-6.88 (m, 7H).

¹³C-NMR (50 MHz, CDCl₃): 12.1, 14.5, 33.4, 42.8, 49.3, 56.0, 56.0, 56.0, 56.1, 62.8, 72.9, 83.3, 109.1, 111.1, 111.4, 112.0, 118.3, 120.6, 128.4, 132.8, 135.1, 137.9, 147.6, 148.6, 149.1, 149.2, 168.0.

### Comparative Example 36

### 10-45: ((2S,3R,4R)-4-(3,4-dimethoxybenzyl)-2-(3,4-dimethoxyphenyl)tetrahydrofuran-3-yl)methyl cyclopentanecarboxylate (2544)

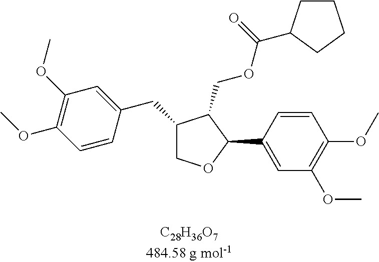

Preparation analogous to that of 10-41, using starting material 9-1 (35.0 mg, 0.090 mmol, 1.00 equiv.), cyclopentanecarboxylic acid (23.6 mg, 0.207 mmol, 2.3 equiv.), EDCI.HCl (34.5 mg, 0.180 mmol, 2.0 equiv.), 4-DMAP (1.1 mg, 9.0 μmol, 0.1 equiv.) and DIPEA (39 μL, 0.23 mmol, 2.5 equiv.).

Work-up and purification: the reaction solution was used directly for flash column chromatography (9 g silica, flow rate 20 mL/min, gradient 10 to 22% EtOAc in LP within 9 min, then isocratically at 22% for 6 min, then 22 to 62% within 30 min) to give a colorless oil (38.7 mg, 89%).

[α]_{D}²⁰: +17.4 (c 2.47, MeOH)

HR-MS (ESI) calc'd for M+Na⁺: 507.2353. found: 507.2358.

¹H-NMR (200 MHz, CDCl₃): δ 1.48-1.97 (m, 8H), 2.47-2.93 (m, 5H), 3.76 (dd, J=8.5, 6.3 Hz, 1H), 3.87 (s, 3H), 3.87 (s, 6H), 3.89 (s, 3H), 4.07 (dd, J=8.6, 6.2 Hz, 1H), 4.18 (dd, J=11.3, 7.0 Hz, 1H), 4.37 (dd, J=11.2, 6.9 Hz, 1H), 4.80 (d, J=6.3 Hz, 1H), 6.66-6.91 (m, 6H).

### Comparative Example 37

### 10-46: ((2S,3R,4R)-4-(3,4-dimethoxybenzyl)-2-(3,4-dimethoxyphenyl)tetrahydrofuran-3-yl)methyl cyclohexanecarboxylate (2545)

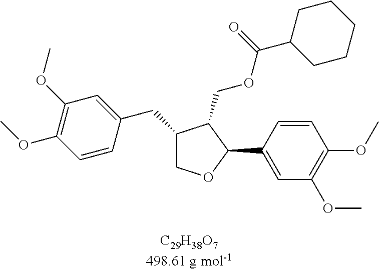

Preparation analogous to that of 10-41, using starting material 9-1 (35.0 mg, 0.090 mmol, 1.00 equiv.), cyclohexanecarboxylic acid (26.5 mg, 0.207 mmol, 2.3 equiv.), EDCI.HCl (34.5 mg, 0.180 mmol, 2.0 equiv.), 4-DMAP (1.1 mg, 9.0 μmol, 0.1 equiv.) and DIPEA (39 μL, 0.23 mmol, 2.5 equiv.).

Work-up and purification: the reaction solution was used directly for flash column chromatography (9 g silica, flow rate 20 mL/min, gradient 10 to 22% EtOAc in LP within 9 min, then isocratically at 22% for 6 min, then 22 to 62% within 30 min) to give a colorless oil (33.3 mg, 74%).

[α]_{D}²⁰: +16.8 (c 1.62, MeOH)

HR-MS (ESI) calc'd for M+Na⁺: 521.2510. found: 521.2516.

¹H-NMR (200 MHz, CDCl₃): δ 1.12-1.96 (m, 10H), 2.18-2.35 (m, 1H), 2.46-2.83 (m, 3H), 2.86 (dd, J=12.4, 4.3 Hz, 1H), 3.76 (dd, J=8.6, 6.2 Hz, 1H), 3.87 (s, 3H), 3.87 (s, 6H), 3.89 (s, 3H), 4.07 (dd, J=8.6, 6.3 Hz, 1H), 4.17 (dd, J=11.3, 6.9 Hz, 1H), 4.37 (dd, J=11.3, 6.9 Hz, 1H), 4.80 (d, J=6.4 Hz, 1H), 6.66-6.91 (m, 6H).

### Example 23

### 10-47: (E)-((2S,3R,4R)-4-(3,4-dimethoxybenzyl)-2-(3,4-dimethoxyphenyl)tetrahydro-furan-3-yl)methyl but-2-enoate (2549)

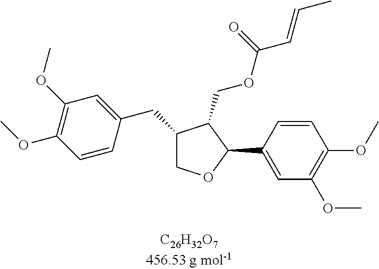

Preparation analogous to that of 10-41, using starting material 9-1 (35.0 mg, 0.090 mmol, 1.00 equiv.) and crotonic acid (31.0 mg, 0.360 mmol, 4.0 equiv.).

Work-up and purification: the reaction solution was used directly for flash column chromatography (9 g silica, flow rate 20 mL/min, gradient 10 to 50% EtOAc in LP within 30 min) to give a nearly colorless oil (32.0 mg, 78%).

[α]_{D}²⁰: +24.6 (c 0.63, MeOH)

HR-MS (ESI) calc'd for M+Na⁺: 479.2040. found: 479.2046.

¹H-NMR (200 MHz, CDCl₃): δ 1.88 (dd, J=6.9, 1.5 Hz, 3H), 2.48-2.94 (m, 4H), 3.75 (dd, J=8.6, 6.3 Hz, 1H), 3.86 (s, 3H), 3.87 (s, 6H), 3.88 (s, 3H), 4.08 (dd, J=8.6, 6.3 Hz, 1H), 4.25 (dd, J=11.3, 7.1 Hz, 1H), 4.43 (dd, J=11.3, 6.9 Hz, 1H), 4.81 (d, J=6.3 Hz, 1H), 5.82 (dd, J=15.6, 1.6 Hz, 1H), 6.66-7.01 (m, 7H).

### Example 24

### 10-52: ((2S,3R,4R)-4-(3,4-dimethoxybenzyl)-2-(3,4-dimethoxyphenyl)tetrahydrofuran-3-yl)methyl cinnamate (2739)

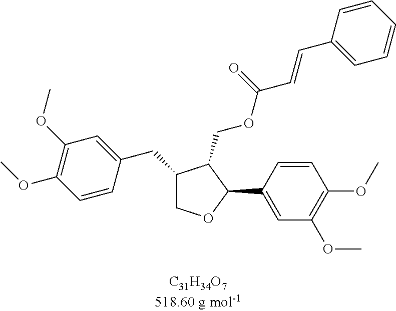 [SMILES: COc1ccc(C[C@H]2CO[C@H](c3ccc(OC)c(OC)c3)[C@H]2COC(=O)/C=C/c2ccccc2)cc1OC]

Preparation analogous to that of 10-41, using starting material 9-1 (35.0 mg, 0.090 mmol, 1.00 equiv.) and cinnamic acid (53.3 mg, 0.360 mmol, 4.0 equiv.).

Work-up and purification: the reaction solution was used directly for flash column chromatography (9 g silica, flow rate 20 mL/min, gradient 10 to 22% EtOAc in LP within 9 min, then isocratically at 22% for 6 min, then 22 to 62% within 30 min) to give a colorless oil (45.1 mg, 97%).

[α]_{D}²⁵: +35.4 (c 4.24, MeOH)

HR-MS (ESI) calc'd for M+Na⁺: 541.2197. found: 541.2205.

¹H-NMR (200 MHz, CDCl₃): δ 2.50-3.00 (m, 4H), 3.78 (dd, J=8.6, 5.8 Hz, 1H), 3.83 (s, 3H), 3.85 (s, 3H), 3.86 (s, 3H), 3.88 (s, 3H), 4.11 (dd, J=8.6, 6.1 Hz, 1H), 4.34 (dd, J=11.3, 7.2 Hz, 1H), 4.52 (dd, J=11.3, 6.6 Hz, 1H), 4.85 (d, J=6.4 Hz, 1H), 6.38 (d, J=16.1 Hz, 1H), 6.68-6.95 (m, 6H), 7.35-7.44 (m, 3H), 7.44-7.54 (m, 2H), 7.57 (d, J=16.1 Hz, 1H).

### Example 25

### 10-55: ((2S,3R,4R)-4-(3,4-dimethoxybenzyl)-2-(3,4-dimethoxyphenyl)tetrahydrofuran-3-yl)methyl 3-methylbut-2-enoate (2738)

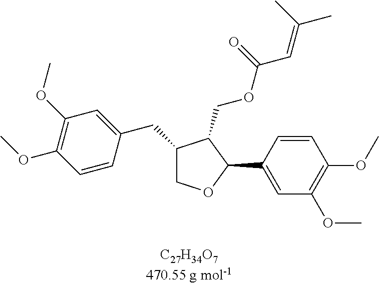

Preparation analogous to that of 10-41, using starting material 9-1 (35.0 mg, 0.090 mmol) and 3-methylbut-2-enoic acid (36.0 mg, 0.360 mmol).

Work-up, post-treatment and purification: the reaction solution was used directly for flash column chromatography (9 g silica, flow rate 20 mL/min, gradient 10 to 22% EtOAc in LP within 9 min, then isocratically at 22% for 6 min, then 22 to 62% within 30 min) to give a mixture of target and isomerization compound (34.4 mg, the corresponding 3-methylbut-3-enoate). Thus, a new reaction vessel was charged with a stir bar and part of the so obtained material (24.7 mg, 0.052 mmol), evacuated and back-flushed with argon. To this was then added tert-BuOK (2.9 mg, 0.026 mmol) in dry THF (1.0 mL) via syringe and the solution stirred at room temperature for 18 h. Then the vessel was opened, there was added THF (1.0 mL), followed by Et₂O (15 mL) and a solution of KHSO₄ (0.029 mmol, 3.9 mg) in saturated brine (2 mL). Water (1.5 mL) was added to dissolve the salts, the layers were separated, the aqueous phase reextracted with Et₂O (2×10 mL), the combined organic phases dried over Na₂SO₄ and the solvents evaporated. The target compound was finally obtained by flash column chromatography (9 g silica, flow rate 20 mL/min, gradient 10 to 50% EtOAc in LP over 30 min) to give a colorless oil (17.0 mg, 40% with respect to starting material 9-1, 56% with respect to mixture applied to post-treatment).

[α]_{D}²⁰: +29.2 (c 1.63, MeOH)

HR-MS (ESI) calc'd for M+Na⁺: 493.2197. found: 493.2201.

¹H-NMR (200 MHz, CDCl₃): δ 1.90 (d, J=1.0 Hz, 3H), 2.17 (d, J=1.1 Hz, 3H), 2.47-2.84 (m, 3H), 2.89 (dd, J=12.7, 4.3 Hz, 1H), 3.75 (dd, J=8.6, 6.3 Hz, 1H), 3.86 (s, 3H), 3.87 (s, 6H), 3.88 (s, 3H), 4.07 (dd, J=8.4, 6.1 Hz, 1H), 4.21 (dd, J=11.4, 7.0 Hz, 1H), 4.37 (dd, J=11.3, 7.0 Hz, 1H), 4.81 (d, J=6.3 Hz, 1H), 5.63-5.68 (m, 1H), 6.66-6.81 (m, 3H), 6.81-6.92 (m, 3H).

### Example 26

### 10-56: ((2S,3R,4R)-4-(3,4-dimethoxybenzyl)-2-phenyltetrahydrofuran-3-yl)methyl cyclopentanecarboxylate (2742)

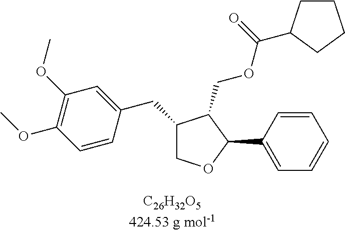 [SMILES: COc1ccc(C[C@H]2CO[C@H](c3ccccc3)[C@H]2COC(=O)C2CCCC2)cc1OC]

Preparation analogous to that of 10-41, using starting material 9-39 (20.0 mg, 0.061 mmol, 1.00 equiv.), cyclopentanecarboxylic acid (27.8 mg, 0.244 mmol, 4.0 equiv.), EDCI.HCl (23.3 mg, 0.122 mmol, 2.0 equiv.), 4-DMAP (0.7 mg, 6.1 μmol, 0.1 equiv.) and DIPEA (25 μL, 0.15 mmol, 2.5 equiv.).

Work-up and purification: the reaction solution was used directly for flash column chromatography (9 g silica, flow rate 20 mL/min, gradient 10 to 15% EtOAc in LP within 10 min, then 15 to 22% within 5 min, then 22 to 60% within 15 min) to give a colorless oil (22.3 mg, 86%).

[α]_{D}²⁵: +16.7 (c 2.20, MeOH)

HR-MS (ESI) calc'd for M+Na⁺: 447.2142. found: 447.2148.

¹H-NMR (200 MHz, CDCl₃): δ 1.47-1.99 (m, 8H), 2.48-2.91 (m, 5H), 3.78 (dd, J=8.5, 6.4 Hz, 1H), 3.86 (s, 3H), 3.87 (s, 3H), 4.09 (dd, J=8.6, 6.3 Hz, 1H), 4.19 (dd, J=11.2, 7.2 Hz, 1H), 4.39 (dd, J=11.2, 6.7 Hz, 1H), 4.89 (d, J=5.9 Hz, 1H), 6.65-6.75 (m, 2H), 6.80 (d, J=7.9 Hz, 1H), 7.40-7.21 (m, 5H).

### Example 27

### 10-59: ((2S,3R,4R)-4-(3,4-dimethoxybenzyl)-2-(3,4-dimethoxyphenyl)tetrahydrofuran-3-yl) methyl 2-(adamantan-1-yl)acetate (2745)

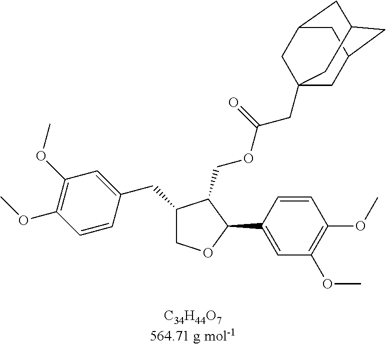 [SMILES: COc1ccc(C[C@H]2CO[C@H](c3ccc(OC)c(OC)c3)[C@H]2COC(=O)CC23CC4CC(CC(C4)C2)C3)cc1OC]

Preparation analogous to that of 10-41, using starting material 9-1 (35.0 mg, 0.090 mmol, 1.00 equiv.), 2-(adamantan-1-yl)acetic acid (40.2 mg, 0.207 mmol, 2.3 equiv.), EDCI.HCl (34.5 mg, 0.180 mmol, 2.0 equiv.), 4-DMAP (1.1 mg, 9.0 μmol, 0.1 equiv.) and DIPEA (39 μL, 0.23 mmol, 2.5 equiv.).

Work-up and purification: the reaction solution was used directly for flash column chromatography (9 g silica, flow rate 20 mL/min, gradient 10 to 15% EtOAc in LP within 10 min, then 15 to 25% within 5 min, then 25 to 48% within 7 min) to give a colorless oil (40.5 mg, 80%).

[α]_{D}²⁵: +18.3 (c 4.05, MeOH)

HR-MS (ESI) calc'd for M+Na⁺: 587.2979. found: 587.3000.

¹H-NMR (200 MHz, CDCl₃): δ 1.52-1.77 (m, 12H), 1.89-2.03 (m, 3H), 2.07 (s, 2H), 2.46-2.64 (m, 2H), 2.64-2.81 (m, 1H), 2.89 (dd, J=12.7, 4.2 Hz, 1H), 3.76 (dd, J=8.6, 6.2 Hz, 1H), 3.86 (s, 3H), 3.87 (s, 6H), 3.89 (s, 3H), 4.06 (dd, J=8.6, 6.3 Hz, 1H), 4.16 (dd, J=11.3, 7.0 Hz, 1H), 4.36 (dd, J=11.2, 7.1 Hz, 1H), 4.81 (d, J=6.3 Hz, 1H), 6.65-6.91 (m, 6H).

### Example 28

### 10-60: ((2S,3R,4R)-4-(3,4-dimethoxybenzyl)-2-phenyltetrahydrofuran-3-yl)methyl cyclohexanecarboxylate (2746)

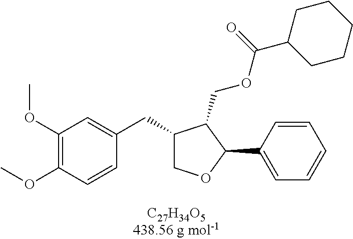 [SMILES: COc1ccc(C[C@H]2CO[C@H](c3ccccc3)[C@H]2COC(=O)C2CCCCC2)cc1OC]

Preparation analogous to that of 10-41, using starting material 9-39 (19.4 mg, 0.059 mmol, 1.00 equiv.), cyclohexanecarboxylic acid (30.3 mg, 0.236 mmol, 4.0 equiv.), EDCI.HCl (22.7 mg, 0.118 mmol, 2.0 equiv.), 4-DMAP (0.7 mg, 5.9 μmol, 0.1 equiv.) and DIPEA (25 μL, 0.15 mmol, 2.5 equiv.).

Work-up and purification: the reaction solution was used directly for flash column chromatography (9 g silica, flow rate 20 mL/min, gradient 10 to 12% EtOAc in LP within 10 min, then 12 to 19% within 5 min, then 19 to 57% within 15 min) to give a colorless oil (21.7 mg, 84%).

[α]_{D}²⁵: +17.9 (c 2.10, MeOH)

HR-MS (ESI) calc'd for M+Na⁺: 461.2298. found: 461.2302.

¹H-NMR (200 MHz, CDCl₃): δ 1.15-1.96 (m, 10H), 2.18-2.35 (m, 1H), 2.47-2.91 (m, 4H), 3.78 (dd, J=8.5, 6.3 Hz, 1H), 3.86 (s, 6H), 4.09 (dd, J=8.5, 6.2 Hz, 1H), 4.18 (dd, J=11.2, 7.2 Hz, 1H), 4.39 (dd, J=11.2, 6.8 Hz, 1H), 4.88 (d, J=6.0 Hz, 1H), 6.65-6.75 (m, 2H), 6.80 (d, J=7.9 Hz, 1H), 7.22-7.40 (m, 5H).

### Example 29

### 10-62: ((2S,3R,4R)-4-(3,4-dimethoxybenzyl)-2-(4-fluorophenyl)tetrahydrofuran-3-yl)-methyl cyclopentanecarboxylate (2781)

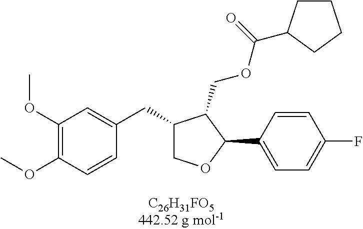 [SMILES: COc1ccc(C[C@H]2CO[C@H](c3ccc(F)cc3)[C@H]2COC(=O)C2CCCC2)cc1OC]

Preparation analogous to that of 10-41, using starting material 9-24 (31.2 mg, 0.090 mmol, 1.00 equiv.), cyclopentanecarboxylic acid (23.6 mg, 0.207 mmol, 2.3 equiv.), EDCI.HCl (34.5 mg, 0.180 mmol, 2.0 equiv.), 4-DMAP (1.1 mg, 9.0 μmol, 0.1 equiv.) and DIPEA (39 μL, 0.23 mmol, 2.5 equiv.).

Work-up and purification: the reaction solution was used directly for flash column chromatography (9 g silica, flow rate 20 mL/min, gradient 0 to 15% EtOAc in LP within 16 min, then 15 to 22% within 4 min) to give a colorless oil (38.2 mg, 96%).

[α]_{D}²⁰: +13.8 (c 2.46, MeOH)

HR-MS (ESI) calc'd for M+Na⁺: 465.2048. found: 465.2024.

¹H-NMR (200 MHz, CDCl₃): δ 1.47-1.98 (m, 8H), 2.44-2.79 (m, 4H), 2.85 (dd, J=12.4, 4.3 Hz, 1H), 3.76 (dd, J=8.5, 6.4 Hz, 1H), 3.86 (s, 3H), 3.87 (s, 3H), 4.07 (dd, J=8.6, 6.2 Hz, 1H), 4.18 (dd, J=11.2, 7.4 Hz, 1H), 4.38 (dd, J=11.2, 6.6 Hz, 1H), 4.85 (d, J=6.0 Hz, 1H), 6.65-6.75 (m, 2H), 6.80 (d, J=7.9 Hz, 1H), 7.02 (t, J=8.7 Hz, 2H), 7.29 (dd, J=8.6, 5.4 Hz, 2H).

### Comparative Example 38

### 10-63: ((2S,3R,4R)-4-(3,4-dimethoxybenzyl)-2-(3,4-dimethoxyphenyl)tetrahydrofuran-3-yl)methyl 2-naphthoate (2784)

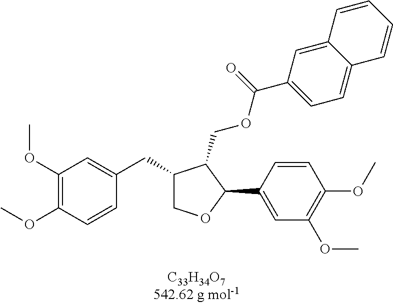 [SMILES: COc1ccc(C[C@H]2CO[C@H](c3ccc(OC)c(OC)c3)[C@H]2COC(=O)c2ccc3ccccc3c2)cc1OC]

Preparation analogous to that of 10-41, using starting material 9-1 (35.0 mg, 0.090 mmol, 1.00 equiv.) and 2-naphthoic acid (62.0 mg, 0.360 mmol, 4.0 equiv.).

Work-up and purification: the reaction solution was used directly for flash column chromatography (9 g silica, flow rate 20 mL/min, gradient 8 to 50% EtOAc in LP within 30 min) to give a colorless oil (43.7 mg, 90%).

[α]_{D}²⁰: +17.6 (c 2.38, MeOH)

HR-MS (APCI-PI) calc'd for M+Na⁺: 565.2197. found: 565.2211.

¹H-NMR (200 MHz, CDCl₃): δ 2.58-3.04 (m, 4H), 3.81 (s, 3H), 3.81-3.90 (m, 1H), 3.83 (s, 3H), 3.84 (s, 3H), 3.84 (s, 3H), 4.15 (dd, J=8.5, 6.0 Hz, 1H), 4.52 (dd, J=11.2, 7.3 Hz, 1H), 4.71 (dd, J=11.2, 6.0 Hz, 1H), 4.94 (d, J=6.2 Hz, 1H), 6.77 (m, 4H), 6.87-6.98 (m, 2H), 7.49-7.65 (m, 2H), 7.80-7.98 (m, 4H), 8.40 (s, 1H).

### Comparative Example 39

### 10-66: ((2S,3R,4R)-4-(4-methylbenzyl)-2-(3,4,5-trim ethoxyphenyl)tetrahydrofuran-3-yl)methyl cyclopentanecarboxylate (2866)

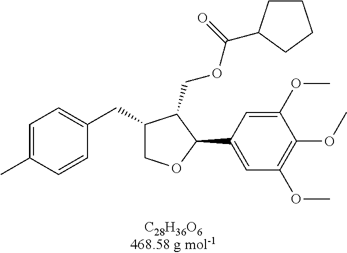

Preparation analogous to that of 10-41, using starting material 9-33 (10.4 mg, 0.028 mmol, 1.00 equiv.), cyclopentanecarboxylic acid (7.3 mg, 0.064 mmol, 2.3 equiv.), EDCI.HCl (10.7 mg, 0.056 mmol, 2.0 equiv.), 4-DMAP (0.3 mg, 2.8 μmol, 0.1 equiv.) and DIPEA (12 μL, 0.07 mmol, 2.5 equiv.), and stirring the reaction for 24 in place of 16 h.

Work-up and purification: the reaction solution was used directly for flash column chromatography (9 g silica, flow rate 20 mL/min, isocratically at 10% EtOAc in LP, then gradient 10 to 15% within 10 min, then 15 to 25% within 5 min, then 25 to 50% within 7 min) to give a colorless oil (7.2 mg, 55%).

¹H-NMR (200 MHz, CDCl₃): δ 1.48-1.98 (m, 8H), 2.33 (s, 3H), 2.48-2.94 (m, 5H), 3.75 (dd, J=8.5, 6.6 Hz, 1H), 3.83 (s, 3H), 3.86 (s, 6H), 4.07 (dd, J=8.6, 6.3 Hz, 1H), 4.20 (dd, J=11.2, 7.0 Hz, 1H), 4.38 (dd, J=11.3, 6.7 Hz, 1H), 4.79 (d, J=5.9 Hz, 1H), 6.54 (s, 2H), 7.03-7.16 (m, 4H).

¹³C-NMR (50 MHz, CDCl₃): δ 21.1, 25.9, 30.1, 33.2, 42.5, 44.0, 49.3, 56.3, 61.0, 62.7, 73.0, 83.3, 102.7, 128.6, 129.4, 136.0, 137.1, 137.4, 138.4, 153.4, 176.7.

### Example 30

### 10-67: ((2S,3R,4R)-4-(4-(tert-butyl)benzyl)-2-(3, 4-dimethoxyphenyl)tetrahydrofuran-3-yl)methyl cyclopentanecarboxylate (2867)

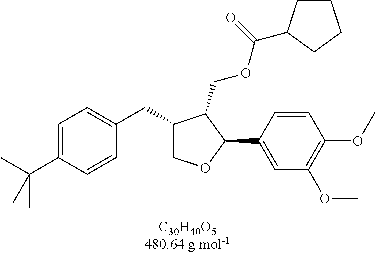 [SMILES: COc1ccc([C@H]2OC[C@H](Cc3ccc(C(C)(C)C)cc3)[C@@H]2COC(=O)C2CCCC2)cc1OC]

Preparation analogous to that of 10-41, using starting material 9-5 (16.7 mg, 0.043 mmol, 1.00 equiv.), cyclopentanecarboxylic acid (11.4 mg, 0.100 mmol, 2.3 equiv.), EDCI.HCl (16.6 mg, 0.087 mmol, 2.0 equiv.), 4-DMAP (0.5 mg, 4.3 μmol, 0.1 equiv.) and DIPEA (19 μL, 0.11 mmol, 2.5 equiv.).

Work-up and purification: the reaction solution was used directly for flash column chromatography (9 g silica, flow rate 20 mL/min, isocratically 3% EtOAc in LP for 3 min, then gradient 3 to 30% within 30 min) to give a colorless oil (16.6 mg, 79%).

[α]_{D}²⁵: +14.2 (c 1.07, iPrOH)

HR-MS (APCI-PI) calc'd for M+Na⁺: 503.2768. found: 503.2751.

¹H-NMR (200 MHz, CDCl₃): δ 1.31 (s, 9H), 1.48-1.96 (m, 8H), 2.48-2.96 (m, 5H), 3.75 (dd, J=8.5, 6.5 Hz, 1H), 3.87 (s, 3H), 3.89 (s, 3H), 4.08 (dd, J=8.6, 6.4 Hz, 1H), 4.19 (dd, J=11.3, 6.9 Hz, 1H), 4.36 (dd, J=11.2, 6.9 Hz, 1H), 4.80 (d, J=6.3 Hz, 1H), 6.78-6.92 (m, 3H), 7.10 (d, J=8.3 Hz, 2H), 7.32 (d, J=8.3 Hz, 2H).

### Comparative Example 40

### 10-69: ((2S,3R,4R)-2-(3,4-dimethoxyphenyl)-4-(4-(trifluoromethyl)benzyl)tetrahydro-furan-3-yl)methyl 2-methylbenzoate (3027)

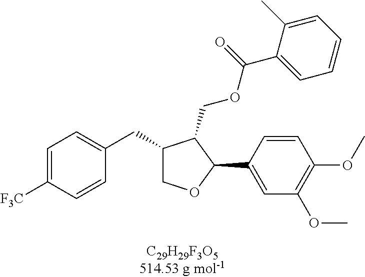 [SMILES: COc1ccc([C@H]2OC[C@H](Cc3ccc(C(F)(F)F)cc3)[C@@H]2COC(=O)c2ccccc2C)cc1OC]

Preparation analogous to that of 10-41, using starting material 9-17 (33.6 mg, 0.085 mmol, 1.00 equiv.) and 2-methylbenzoic acid (46.1 mg, 0.339 mmol, 4.0 equiv.).

Work-up and purification: the reaction solution was used directly for flash column chromatography (9 g silica, flow rate 20 mL/min, gradient 5 to 40% EtOAc in heptane within 40 min) to give a colorless oil (27.6 mg, 63%).

[α]_{D}²⁵: +17.0 (c 0.83, iPrOH)

¹H-NMR (200 MHz, CDCl₃): δ 2.59 (s, 3H), 2.65-2.96 (m, 3H), 3.05 (dd, J=12.5, 3.6 Hz, 1H), 3.77 (dd, J=8.8, 5.8 Hz, 1H), 3.83 (s, 3H), 3.86 (s, 3H), 4.11 (dd, J=8.8, 6.0 Hz, 1H), 4.43 (dd, J=11.3, 6.9 Hz, 1H), 4.58 (dd, J=11.3, 6.8 Hz, 1H), 4.91 (d, J=6.3 Hz, 1H), 6.78-6.93 (m, 3H), 7.15-7.35 (m, 4H), 7.41 (td, J=7.4, 1.5 Hz, 1H), 7.55 (d, J=8.1 Hz, 2H), 7.76 (dd, J=7.9, 1.4 Hz, 1H).

¹³C-NMR (50 MHz, CDCl₃): δ 21.9, 33.7, 42.4, 49.4, 56.0, 56.1, 62.9, 72.7, 83.3, 109.1, 111.2, 118.3, 125.7 (q, J=3.7 Hz), 125.9, 128.9 (q, J=32.5 Hz), 129.1, 130.6, 132.0, 132.4, 134.7, 140.6, 144.3, 148.7, 149.3, 167.3. Two C_{q} not visible.

### Comparative Example 41

### 10-70: ((2S,3R,4R)-4-(3,4-dimethoxybenzyl)-2-(3,4-dimethoxyphenyl)tetrahydrofuran-3-yl)methyl 3-methylbutanoate (2854)

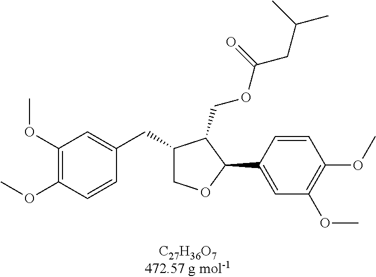

Preparation analogous to that of 10-41, using starting material 9-1 (22.8 mg, 0.059 mmol, 1.00 equiv.) and 3-methylbutanoic acid (13.8 mg, 0.135 mmol, 2.3 equiv.), EDCI.HCl (22.5 mg, 0.117 mmol, 2.0 equiv.), 4-DMAP (0.7 mg, 5.9 μmol, 0.1 equiv.) and DIPEA (26 μL, 0.15 mmol, 2.5 equiv.).

Work-up and purification: the reaction solution was used directly for flash column chromatography (9 g silica, flow rate 20 mL/min, gradient 10 to 30% EtOAc in LP within 30 min) to give a colorless oil (22.0 mg, 96%).

[α]_{D}²³: +22.9 (c 0.90, MeOH)

HR-MS (APCI-PI) calc'd for M+Na⁺: 495.2353. found: 495.2371.

¹H-NMR (200 MHz, CDCl₃): δ 0.96 (d, J=6.4 Hz, 6H), 1.97-2.22 (m, 3H), 2.47-2.64 (m, 2H), 2.64-2.81 (m, 1H), 2.87 (dd, J=12.5, 4.3 Hz, 1H), 3.75 (dd, J=8.6, 6.2 Hz, 1H), 3.86 (s, 3H), 3.87 (s, 6H), 3.89 (s, 3H), 4.07 (dd, J=8.6, 6.3 Hz, 1H), 4.18 (dd, J=11.3, 7.1 Hz, 1H), 4.38 (dd, J=11.2, 7.0 Hz, 1H), 4.79 (d, J=6.4 Hz, 1H), 6.66-6.90 (m, 6H).

### Comparative Example 42

### 10-71: ((2S,3R,4R)-4-(3,4-dimethoxybenzyl)-2-(3,4-dimethoxyphenyl)tetrahydrofuran-3-yl)methyl 3,3-dimethylbutanoate (2855)

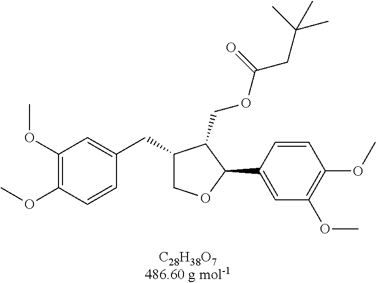

Preparation analogous to that of 10-41, using starting material 9-1 (30.0 mg, 0.077 mmol, 1.00 equiv.) and 3,3-dimethylbutanoic acid (20.6 mg, 0.178 mmol, 2.3 equiv.), EDCI.HCl (29.6 mg, 0.154 mmol, 2.0 equiv.), 4-DMAP (0.9 mg, 7.7 μmol, 0.1 equiv.) and DIPEA (34 μL, 0.19 mmol, 2.5 equiv.).

Work-up and purification: the reaction solution was used directly for flash column chromatography (9 g silica, flow rate 20 mL/min, gradient 10 to 30% EtOAc in LP within 30 min) to give a colorless oil (19.5 mg, 51%).

[α]_{D}²³: +24.7 (c 1.01, MeOH)

HR-MS (APCI-PI) calc'd for M+Na⁺: 509.2510. found: 509.2512.

¹H-NMR (200 MHz, CDCl₃): δ 1.03 (d, J=5.5 Hz, 9H), 2.20 (s, 2H), 2.47-2.63 (m, 2H), 2.64-2.82 (m, 1H), 2.88 (dd, J=12.5, 4.2 Hz, 1H), 3.76 (dd, J=8.6, 6.1 Hz, 1H), 3.86 (s, 3H), 3.87 (s, 3H), 3.87 (s, 3H), 3.89 (s, 3H), 4.06 (dd, J=8.6, 6.2 Hz, 1H), 4.16 (dd, J=11.3, 6.9 Hz, 1H), 4.36 (dd, J=11.3, 7.2 Hz, 1H), 4.80 (d, J=6.4 Hz, 1H), 6.66-6.90 (m, 6H).

### Example 31

### 10-72: ((2S,3R,4R)-4-(3,4-dimethoxybenzyl)-2-(4-fluorophenyl)tetrahydrofuran-3-yl)-methyl 2-ethylbutanoate (2856)

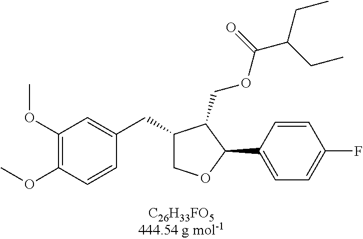 [SMILES: CCC(CC)C(=O)OC[C@H]1[C@@H](Cc2ccc(OC)c(OC)c2)CO[C@@H]1c1ccc(F)cc1]

Preparation analogous to that of 10-41, using starting material 9-24 (15.2 mg, 0.044 mmol, 1.00 equiv.) and 2-ethylbutanoic acid (11.7 mg, 0.101 mmol, 2.3 equiv.), EDCI.HCl (16.8 mg, 0.088 mmol, 2.0 equiv.), 4-DMAP (0.5 mg, 4.4 μmol, 0.1 equiv.) and DIPEA (19 μL, 0.110 mmol, 2.5 equiv.), and stirring for 13 in place of 16 h.

Work-up and purification: the reaction solution was used directly for flash column chromatography (9 g silica, flow rate 20 mL/min, gradient 0 to 10% EtOAc in LP within 60 min) to give a colorless oil (16.2 mg, 83%).

[α]_{D}²³: +14.2 (c 0.65, MeOH)

HR-MS (APCI-PI) calc'd for M+Na⁺: 467.2204. found: 467.2197.

¹H-NMR (200 MHz, CDCl₃): δ 0.89 (t, J=7.4 Hz, 3H), 0.90 (t, J=7.4 Hz, 3H), 1.44-1.70 (m, 4H), 2.14-2.29 (m, 1H), 2.45-2.61 (m, 2H), 2.62-2.81 (m, 1H), 2.87 (dd, J=12.5, 4.1 Hz, 1H), 3.77 (dd, J=8.6, 6.3 Hz, 1H), 3.86 (s, 6H), 4.06 (dd, J=8.6, 6.2 Hz, 1H), 4.17 (dd, J=11.2, 7.2 Hz, 1H), 4.41 (dd, J=11.3, 6.7 Hz, 1H), 4.85 (d, J=6.1 Hz, 1H), 6.65-6.84 (m, 3H), 6.97-7.09 (m, 2H), 7.24-7.34 (m, 2H).

### Comparative Example 43

### 10-75: ((2S,3R,4R)-4-(3,4-dimethoxybenzyl)-2-(4-fluorophenyl)tetrahydrofuran-3-yl)-methyl 3,3-dimethylbutanoate (2861)

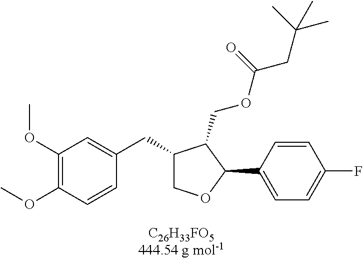 [SMILES: COc1ccc(C[C@H]2CO[C@H](c3ccc(F)cc3)[C@H]2COC(=O)CC(C)(C)C)cc1OC]

Preparation analogous to that of 10-41, using starting material 9-24 (22.0 mg, 0.064 mmol, 1.00 equiv.) and 3,3-dimethylbutanoic acid (16.9 mg, 0.146 mmol, 2.3 equiv.), EDCI.HCl (24.3 mg, 0.127 mmol, 2.0 equiv.), 4-DMAP (0.8 mg, 6.4 μmol, 0.1 equiv.) and DIPEA (28 μL, 0.16 mmol, 2.5 equiv.), and stirring for 13.5 in place of 16 h.

Work-up and purification: the reaction solution was used directly for flash column chromatography (9 g silica, flow rate 20 mL/min, gradient 9 to 25% EtOAc in LP within 60 min) to give a colorless oil (25.8 mg, 92%).

[α]_{D}²³: +15.7 (c 2.07, MeOH)

HR-MS (APCI-PI) calc'd for M+H+: 445.2385. found: 445.2398.

¹H-NMR (200 MHz, CDCl₃): δ 1.03 (s, 9H), 2.19 (s, 2H), 2.43-2.62 (m, 2H), 2.62-2.82 (m, 1H), 2.86 (dd, J=12.5, 4.2 Hz, 1H), 3.77 (dd, J=8.6, 6.3 Hz, 1H), 3.86 (s, 3H), 3.86 (s, 3H), 4.06 (dd, J=8.6, 6.2 Hz, 1H), 4.16 (dd, J=11.1, 7.2 Hz, 1H), 4.37 (dd, J=11.3, 6.9 Hz, 1H), 4.85 (d, J=6.1 Hz, 1H), 6.64-6.84 (m, 3H), 6.96-7.09 (m, 2H), 7.23-7.34 (m, 2H).

### Comparative Example 44

### 10-76: ((2S,3R,4R)-4-(3,4-dimethoxybenzyl)-2-(3,4-dimethoxyphenyl)tetrahydrofuran-3-yl)methyl 2-ethylbutanoate (2860)

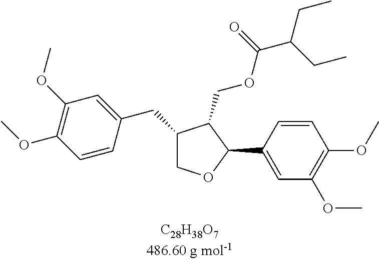

Preparation analogous to that of 10-41, using starting material 9-1 (20.3 mg, 0.052 mmol, 1.00 equiv.) and 2-ethylbutanoic acid (14.0 mg, 0.120 mmol, 2.3 equiv.), EDCI.HCl (20.0 mg, 0.105 mmol, 2.0 equiv.), 4-DMAP (0.6 mg, 5.2 μmol, 0.1 equiv.) and DIPEA (23 μL, 0.13 mmol, 2.5 equiv.).

Work-up and purification: the reaction solution was used directly for flash column chromatography (9 g silica, flow rate 20 mL/min, gradient 10 to 30% EtOAc in LP within 30 min) to give a colorless oil (22.2 mg, 87%).

[α]_{D}²³: +23.3 (c 0.88, MeOH)

HR-MS (APCI-PI) calc'd for M+Na⁺: 509.2510. found: 509.2533.

¹H-NMR (200 MHz, CDCl₃): δ 0.90 (t, J=7.4 Hz, 6H), 1.42-1.75 (m, 4H), 2.15-2.30 (m, 1H), 2.47-2.82 (m, 3H), 2.88 (dd, J=12.6, 4.0 Hz, 1H), 3.76 (dd, J=8.6, 6.1 Hz, 1H), 3.87 (s, 6H), 3.87 (s, 3H), 3.89 (s, 3H), 4.06 (dd, J=8.6, 6.2 Hz, 1H), 4.18 (dd, J=11.3, 6.8 Hz, 1H), 4.41 (dd, J=11.3, 7.1 Hz, 1H), 4.81 (d, J=6.4 Hz, 1H), 6.64-6.91 (m, 6H).

### Example 32

### 10-77: ((2S,3R,4R)-4-(3,4-dimethoxybenzyl)-2-(4-fluorophenyl)tetrahydrofuran-3-yl)-methyl 3-methylbutanoate (2862)

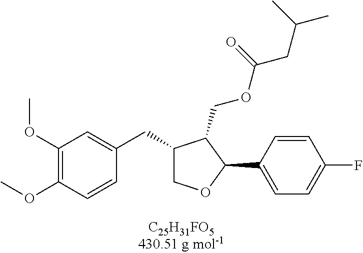 [SMILES: COc1ccc(C[C@H]2CO[C@H](c3ccc(F)cc3)[C@H]2COC(=O)CC(C)C)cc1OC]

Preparation analogous to that of 10-41, using starting material 9-24 (23.7 mg, 0.068 mmol, 1.00 equiv.) and 3-methylbutanoic acid (16.1 mg, 0.157 mmol, 2.3 equiv.), EDCI.HCl (26.2 mg, 0.137 mmol, 2.0 equiv.), 4-DMAP (0.8 mg, 6.8 μmol, 0.1 equiv.) and DIPEA (30 μL, 0.17 mmol, 2.5 equiv.), and stirring for 24 in place of 16 h.

Work-up and purification: the reaction solution was used directly for flash column chromatography (9 g silica, flow rate 20 mL/min, gradient 9 to 25% EtOAc in LP within 60 min) to give a colorless oil (18.6 mg, 63%).

[α]_{D}²³: +15.6 (c 0.48, MeOH)

HR-MS (APCI-PI) calc'd for M+H+: 431.2228. found: 431.2247.

¹H-NMR (200 MHz, CDCl₃): δ 0.95 (d, J=6.3 Hz, 6H), 1.96-2.21 (m, 3H), 2.43-2.62 (m, 2H), 2.63-2.80 (m, 1H), 2.85 (dd, J=12.4, 4.3 Hz, 1H), 3.76 (dd, J=8.6, 6.4 Hz, 1H), 3.86 (s, 3H), 3.87 (s, 3H), 4.07 (dd, J=8.6, 6.2 Hz, 1H), 4.18 (dd, J=11.2, 7.5 Hz, 1H), 4.39 (dd, J=11.2, 6.7 Hz, 1H), 4.84 (d, J=6.0 Hz, 1H), 6.65-6.84 (m, 3H), 6.96-7.09 (m, 2H), 7.22-7.34 (m, 2H).

### Example 33

### 10-78: ((2S,3R,4R)-4-(3,4-dimethoxybenzyl)-2-(4-fluorophenyl)tetrahydrofuran-3-yl)methyl cyclobutanecarboxylate (2863)

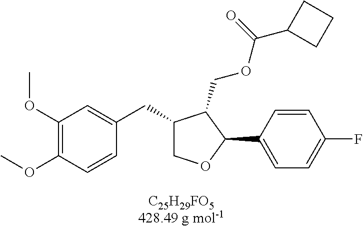 [SMILES: COc1ccc(C[C@H]2CO[C@H](c3ccc(F)cc3)[C@H]2COC(=O)C2CCC2)cc1OC]

Preparation analogous to that of 10-41, using starting material 9-24 (17.0 mg, 0.049 mmol, 1.00 equiv.) and cyclobutanecarboxylic acid (11.3 mg, 0.113 mmol, 2.3 equiv.), EDCI.HCl (18.8 mg, 0.098 mmol, 2.0 equiv.), 4-DMAP (0.6 mg, 4.9 μmol, 0.1 equiv.) and DIPEA (21 μL, 0.12 mmol, 2.5 equiv.), and stirring for 13.5 in place of 16 h.

Work-up and purification: the reaction solution was used directly for flash column chromatography (9 g silica, flow rate 20 mL/min, isocratically at 9% EtOAc in LP for 20 min, then gradient 9 to 25% within 60 min) to give a colorless oil (15.8 mg, 75%).

[α]_{D}²³: +14.2 (c 1.25, MeOH)

HR-MS (APCI-PI) calc'd for M+H+: 429.2072. found: 429.2085.

¹H-NMR (200 MHz, CDCl₃): δ 1.78-2.07 (m, 2H), 2.07-2.36 (m, 4H), 2.44-2.91 (m, 4H), 3.10 (quint, J=8.3 Hz, 1H), 3.75 (dd, J=8.6, 6.4 Hz, 1H), 3.86 (s, 3H), 3.87 (s, 3H), 4.07 (dd, J=8.6, 6.3 Hz, 1H), 4.19 (dd, J=11.2, 7.5 Hz, 1H), 4.39 (dd, J=11.2, 6.6 Hz, 1H), 4.84 (d, J=6.0 Hz, 1H), 6.65-6.85 (m, 3H), 6.95-7.09 (m, 2H), 7.21-7.35 (m, 2H).

### Comparative Example 45

### 10-79: ((2S,3R,4R)-4-(3,4-dimethoxybenzyl)-2-(3,4-dimethoxyphenyl)tetrahydrofuran-3-yl)methyl pivalate (2734)

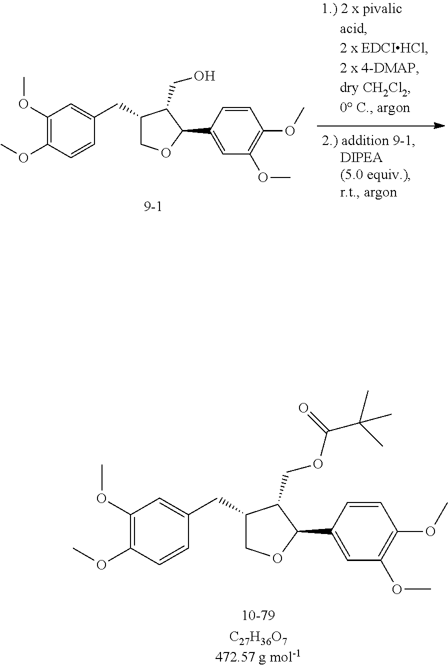

Preparation: a reaction vessel was charged with a stir bar, pivalic acid (21.1 mg, 0.207 mmol, 2.3 equiv.) and 4-DMAP (1.1 mg, 9.0 μmol, 0.1 equiv.), and then evacuated and back-filled with argon using standard Schlenk technique (1×). Then was added dry CH₂Cl₂ (1.0 mL) via syringe and the solution cooled to 0° C. in an ice bath. The vessel was briefly opened, EDCI.HCl (34.5 mg, 0.180 mmol, 2.0 equiv.) added in one go and the mixture stirred for 3 h at 0° C. Meanwhile, a second vessel was charged with a stir bar and 9-1 (35.0 mg, 0.090 mmol, 1.00 equiv.), evacuated and back-filled with argon (3×), and DIPEA (39 μL, 0.23 mmol, 2.5 equiv.) was added via syringe. After 3 h, the solution containing the activated carboxylic acid was transferred to the second vial via syringe and stirred for 70 h at room temperature. To complete the reaction, more of the activated carboxylic acid was prepared in a separate vessel in the fashion as above (using pivalic acid (18.3 mg, 0.180 mmol, 2.0 equiv.), 4-DMAP (1.1 mg, 9.0 μmol, 0.1 equiv.) and EDCI.HCl (30.0 mg, 0.157 mmol, 1.7 equiv.)) and then, after 3 h at 0° C., added to the reaction vial, followed by more DIPEA (39 μL, 0.23 mmol, 2.5 equiv.) via syringe, and the mixture stirred for another 96 h at room temperature.

Work-up and purification: the reaction solution was used directly for flash column chromatography (9 g silica, flow rate 20 mL/min, gradient 10 to 22% EtOAc in LP within 9 min, then isocratically at 22% for 6 min, then 22 to 38% within 12 min, then 38 to 100% within 10 min) to give a colorless oil (32.2 mg, 76%).

[α]_{D}²³: +22.5 (c 2.72, MeOH)

HR-MS (ESI) calc'd for M+Na⁺: 495.2353. found: 495.2351.

¹H-NMR (200 MHz, CDCl₃): δ 1.21 (s, 9H), 2.45-2.81 (m, 3H), 2.87 (dd, J=12.4, 4.1 Hz, 1H), 3.77 (dd, J=8.6, 6.2 Hz, 1H), 3.88 (s, 9H), 3.89 (s, 3H), 4.07 (dd, J=8.6, 6.3 Hz, 1H), 4.17 (dd, J=11.3, 6.8 Hz, 1H), 4.36 (dd, J=11.3, 6.9 Hz, 1H), 4.82 (d, J=6.3 Hz, 1H), 6.66-6.90 (m, 6H).

¹³C-NMR (50 MHz, CDCl₃): δ 27.3, 33.3, 38.9, 42.8, 49.4, 56.0, 56.0, 56.0, 56.1, 62.7, 72.8, 82.9, 108.9, 111.2, 111.4, 112.0, 118.1, 120.6, 132.7, 135.1, 147.6, 148.6, 149.1, 149.2, 178.5.

### Example 34

### 10-80: (E)-((2S,3R,4R)-4-(3,4-dimethoxybenzyl)-2-(3,4,5-trimethoxyphenyl)tetra-hydrofuran-3-yl)methyl 2-methylbut-2-enoate (2628)

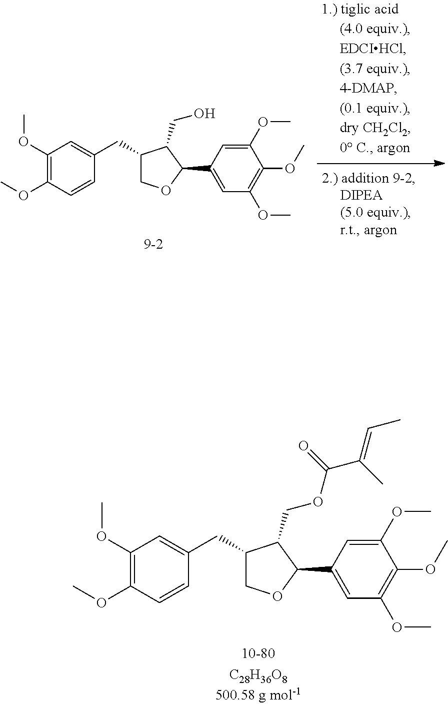

Preparation: a reaction vessel was charged with a stir bar, tiglic acid (29.6 mg, 0.296 mmol, 4.0 equiv.) and 4-DMAP (0.9 mg, 7.4 μmol, 0.1 equiv.), and then evacuated and back-filled with argon using standard Schlenk technique (1×). Then was added dry CH₂Cl₂ such as to achieve a 0.3 M solution (with respect to the carboxylic acid) via syringe and cooled to 0° C. in an ice bath. The vessel was briefly opened, EDCI.HCl (52.5 mg, 0.274 mmol, 3.7 equiv.) added in one go and the mixture stirred for 3 h at 0° C. Meanwhile, a second vessel was charged with a stir bar and 9-2 (30.0 mg, 0.074 mmol, 1.00 equiv.), evacuated and back-filled with argon (3×), and DIPEA (64 μL, 0.37 mmol, 5.0 equiv.) was added via syringe. After 3 h, the solution containing the activated carboxylic acid was transferred to the second vial via syringe and stirred for 20 h at room temperature.

Work-up and purification: the reaction solution was used directly for flash column chromatography (9 g silica, flow rate 20 mL/min, gradient 10 to 22% EtOAc in LP within 10 min, then isocratically at 22% for 6 min, then 22 to 65% within 30 min) to give a slightly yellow, viscous oil (25.4 mg, 70%).

[α]_{D}²⁰: +19.2 (c 1.9, MeOH)

HR-MS (ESI) calc'd for M+Na⁺: 523.2302. found: 523.2311.

¹H-NMR (200 MHz, CDCl₃): δ 1.69-1.85 (m, 6H), 2.46-2.95 (m, 4H), 3.81 (s, 3H), 3.83 (s, 7H), 3.84 (s, 3H), 3.85 (s, 3H), 4.07 (dd, J=8.5, 6.2 Hz, 1H), 4.27 (dd, J=11.3, 7.3 Hz, 1H), 4.43 (dd, J=11.3, 6.4 Hz, 1H), 4.81 (d, J=6.1 Hz, 1H), 6.53 (s, 2H), 6.64-6.84 (m, 4H).

¹³C-NMR (50 MHz, CDCl₃): δ 12.0, 14.4, 33.1, 42.6, 49.1, 55.8, 56.0, 60.8, 62.7, 72.9, 83.4, 102.6, 111.3, 111.8, 120.4, 128.2, 132.5, 137.2, 137.8, 138.2, 147.5, 148.9, 153.2, 167.8.

### Comparative Example 46

### 10-82: ((2S,3R,4R)-4-(3,4-dimethoxybenzyl)-2-(3,4,5-trimethoxyphenyl)tetrahydro-furan-3-yl)methyl cyclohex-1-enecarboxylate (2634)

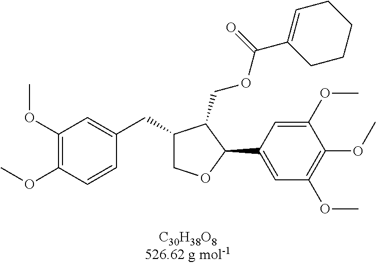

Preparation, work-up and purification identical to that of 10-80 except for using cyclohex-1-enecarboxylic acid to give a slightly yellow, viscous oil (21.3 mg, 56%).

[α]_{D}²⁰: +17.9 (c 1.16, MeOH)

¹H-NMR (200 MHz, CDCl₃): δ 1.49-1.70 (m, 4H), 2.07-2.29 (m, 4H), 2.48-2.93 (m, 4H), 3.69-3.80 (m, 1H), 3.83 (s, 3H), 3.85 (s, 6H), 3.86 (s, 3H), 3.87 (s, 3H), 4.02-4.14 (m, 1H), 4.21-4.34 (m, 1H), 4.37-4.50 (m, 1H), 4.82 (d, J=6.1 Hz, 1H), 6.55 (s, 2H), 6.64-6.83 (m, 3H), 6.90 (m, 1H).

¹³C-NMR (50 MHz, CDCl₃): δ 21.3, 22.0, 24.1, 25.8, 33.1, 42.6, 49.1, 55.8, 55.9, 56.0, 60.8, 62.5, 72.9, 83.5, 102.6, 111.3, 111.8, 120.4, 129.5, 130.3, 132.5, 138.3, 140.4, 147.5, 148.9, 153.2, 167.3.

### Example 35

### 10-83: ((2S,3R,4R)-4-(3,4-dimethoxybenzyl)-2-(3,4,5-trimethoxyphenyl)tetrahydro-furan-3-yl)methyl cyclopent-1-enecarboxylate (2636)

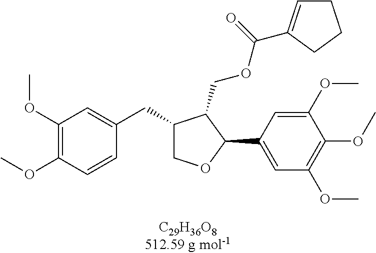

Preparation, work-up and purification identical to that of 10-80 except for using cyclopent-1-enecarboxylic acid to give a slightly yellow, viscous oil (25.7 mg, 69%).

[α]_{D}²⁰: +21.4 (c 1.71, MeOH)

¹H-NMR (200 MHz, CDCl₃): δ 1.85-2.04 (m, 2H), 2.39-2.95 (m, 8H), 3.76 (dd, J=8.6, 6.8 Hz, 1H), 3.81 (s, 3H), 3.84 (s, 6H), 3.85 (s, 3H), 3.86 (s, 3H), 4.08 (dd, J=8.6, 6.5 Hz, 1H), 4.28 (dd, J=11.2, 7.2 Hz, 1H), 4.44 (dd, J=11.2, 6.4 Hz, 1H), 4.81 (d, J=5.9 Hz, 1H), 6.53 (s, 2H), 6.64-6.84 (m, 4H).

¹³C-NMR (50 MHz, CDCl₃): δ 23.0, 31.3, 33.1, 33.4, 42.6, 49.0, 55.8, 55.9, 56.0, 60.8, 62.4, 72.9, 83.4, 102.8, 111.3, 111.8, 120.4, 132.5, 136.2, 137.2, 138.2, 144.5, 147.5, 148.9, 153.2, 165.1.

### Example 36

### 10-84: ((2S,3R,4R)-4-(3,4-dimethoxybenzyl)-2-(3,4,5-trimethoxyphenyl)tetrahydro-furan-3-yl)methyl benzoate (2638)

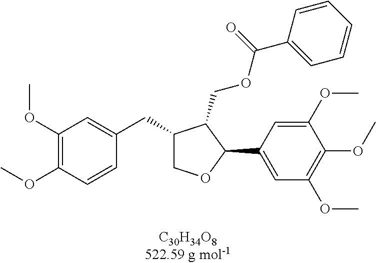

Preparation, work-up and purification identical to that of 10-80 except for using benzoic acid to give a slightly yellow, viscous oil (34.5 mg, 93%).

[α]_{D}²⁰: +23.3 (c 2.17, MeOH)

¹H-NMR (200 MHz, CDCl₃): δ 2.47-2.54 (m, 4H), 3.80 (s, 6H), 3.81 (s, 3H), 3.85 (s, 6H), 4.14 (dd, J=8.0, 6.1 Hz, 1H), 4.48 (dd, J=11.4, 7.3 Hz, 1H), 4.66 (dd, J=11.4, 6.2 Hz, 1H), 4.91 (d, J=5.9 Hz, 1H), 6.57 (s, 2H), 6.64-6.84 (m, 3H), 7.37-7.49 (m, 2H), 7.58 (tt, J=7.3, 1.4 Hz, 1H), 7.90-8.00 (m, 2H).

¹³C-NMR (50 MHz, CDCl₃): δ 33.2, 42.6, 49.1, 55.8, 56.0, 60.8, 63.3, 72.9, 83.6, 102.6, 111.3, 111.8, 120.4, 128.4, 129.5, 129.7, 132.4, 133.3, 137.2, 138.1, 148.9, 147.5, 153.3, 166.3.

### Example 37

### 10-85: ((2S,3R,4R)-4-(3,4-dimethoxybenzyl)-2-(3,4,5-trimethoxyphenyl)tetrahydro-furan-3-yl)methyl butyrate (2629)

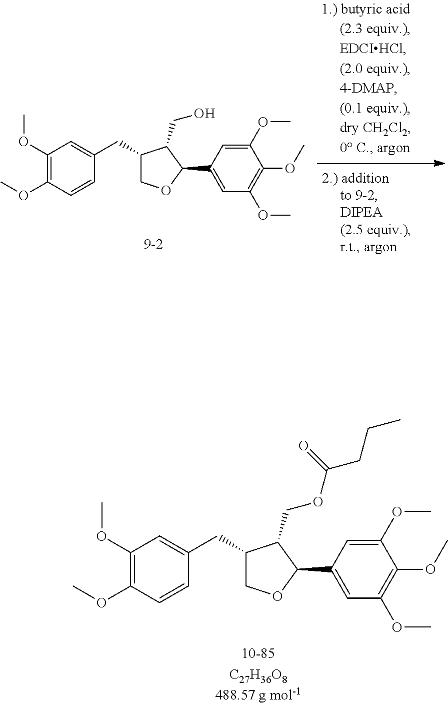

Preparation: a reaction vessel was charged with a stir bar, butyric acid (15.0 mg, 0.170 mmol, 2.3 equiv.) and 4-DMAP (0.9 mg, 7.4 μmol, 0.1 equiv.), and then evacuated and back-filled with argon using standard Schlenk technique (1×). Then was added dry CH₂Cl₂ such as to achieve a 0.1 M solution (with respect to the carboxylic acid) via syringe and cooled to 0° C. in an ice bath. The vessel was briefly opened, EDCI.HCl (28.4 mg, 0.148 mmol, 2.0 equiv.) added in one go and the mixture stirred for 3 h at 0° C. Meanwhile, a second vessel was charged with a stir bar and 9-2 (30.0 mg, 0.074 mmol, 1.00 equiv.), evacuated and back-filled with argon (3×), and DIPEA (32 μL, 0.19 mmol, 2.5 equiv.) was added via syringe. After 3 h, the solution containing the activated carboxylic acid was transferred to the second vial via syringe and stirred for 20 h at room temperature.

Work-up and purification: the reaction solution was used directly for flash column chromatography (9 g silica, flow rate 20 mL/min, gradient 10 to 22% EtOAc in LP within 10 min, then isocratically at 22% for 6 min, then 22 to 65% within 30 min) to give a slightly yellow, viscous oil (25.1 mg, 69%).

[α]_{D}²⁰: +19.2 (c 1.95, MeOH)

HR-MS (ESI) calc'd for M+Na⁺: 511.2302. found: 511.2282.

¹H-NMR (200 MHz, CDCl₃): δ 0.94 (t, J=7.4, 3H), 1.64 (qt, J=7.4, 7.4, 2H), 2.26 (t, J=7.4, 2H), 2.46-2.92 (m, 4H), 3.74 (dd, J=8.6, 6.5 Hz, 1H), 3.82 (s, 3H), 3.85 (s, 9H), 3.88 (s, 3H), 4.06 (dd, J=8.6, 6.3 Hz, 1H), 4.20 (dd, J=11.2, 7.2 Hz, 1H), 4.38 (dd, J=11.2, 6.8 Hz, 1H), 4.77 (d, J=5.9 Hz, 1H), 6.53 (s, 2H), 6.64-6.83 (m, 3H).

¹³C-NMR (50 MHz, CDCl₃): δ 13.7, 18.4, 33.1, 36.2, 42.4, 49.0, 55.9, 56.1, 60.8, 62.5, 72.8, 83.2, 102.6, 111.3, 111.8, 120.4, 132.5, 138.2, 147.5, 148.9, 153.3, 173.5. One C_{q} not visible.

### Example 38

### 10-86: ((2S,3R,4R)-4-(3,4-dimethoxybenzyl)-2-(3,4,5-trimethoxyphenyl)tetrahydro-furan-3-yl)methyl isobutyrate (2632)

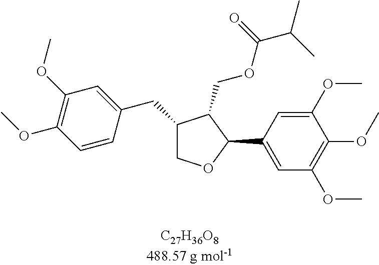

Preparation, work-up and purification identical to that of 10-85 except for using isobutyric acid to give a slightly yellow, viscous oil (29.1 mg, 83%).

[α]_{D}²⁰: +19.9 (c 2.19, MeOH)

¹H-NMR (200 MHz, CDCl₃): δ 1.16 (d, J=6.9 Hz, 6H), 2.45-2.91 (m, 5H), 3.75 (dd, J=8.6, 6.5 Hz, 1H), 3.81 (s, 3H), 3.85 (s, 12H), 4.06 (dd, J=8.6, 6.3 Hz, 1H), 4.19 (dd, J=11.4, 7.1 Hz, 1H), 4.38 (dd, J=11.4, 6.7 Hz, 1H), 4.79 (d, J=5.9 Hz, 1H), 6.53 (s, 2H), 6.63-6.84 (m, 3H).

¹³C-NMR (50 MHz, CDCl₃): δ 18.9, 33.1, 34.0, 42.5, 49.1, 55.8, 56.1, 60.8, 62.5, 72.8, 83.1, 102.5, 111.3, 111.8, 120.4, 132.5, 138.2, 147.5, 148.9, 153.3, 176.8. One C_{q} not visible.

### Example 39

### 10-87: ((2S,3R,4R)-4-(3,4-dimethoxybenzyl)-2-(3,4,5-trimethoxyphenyl)tetrahydro-furan-3-yl)methyl cyclohexanecarboxylate (2633)

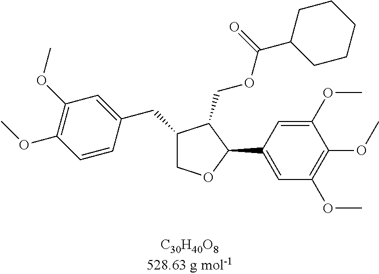

Preparation, work-up and purification identical to that of 10-85 except for using cyclohexanecarboxylic acid to give a slightly yellow, viscous oil (16.5 mg, 42%).

[α]_{D}²⁰: +18.9 (c 2.10, MeOH)

¹H-NMR (200 MHz, CDCl₃): δ 1.13-1.97 (m, 10H), 2.18-2.35 (m, 1H), 2.44-2.93 (m, 4H), 3.75 (dd, J=8.6, 6.7 Hz, 1H), 3.81 (s, 3H), 3.85 (s, 12H), 4.05 (dd, J=8.6, 6.2 Hz, 1H), 4.18 (dd, J=11.2, 7.1 Hz, 1H), 4.38 (dd, J=11.2, 6.7 Hz, 1H), 4.78 (d, J=6.1, 1 H), 6.53 (s, 2H), 6.63-6.84 (m, 3H).

¹³C-NMR (50 MHz, CDCl₃): δ 25.4, 25.7, 29.0, 33.1, 42.5, 43.2, 49.1, 55.8, 55.9, 56.1, 60.8, 62.4, 72.8, 83.1, 102.5, 111.3, 111.8, 120.4, 132.5, 137.2, 138.2, 147.5, 148.9, 153.3, 175.8.

### Example 40

### 10-88: ((2S,3R,4R)-4-(3,4-dimethoxybenzyl)-2-(3,4,5-trimethoxyphenyl)tetrahydro-furan-3-yl)methyl cyclopentanecarboxylate (2635)

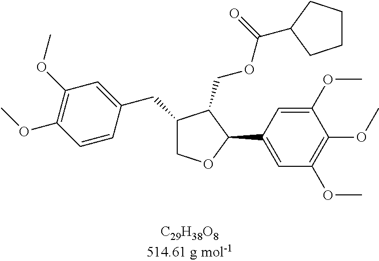

Preparation, work-up and purification identical to that of 10-85 except for using cyclopentanecarboxylic acid to give a slightly yellow, viscous oil (31.4 mg, 85%).

[α]_{D}²⁰: +20.5 (c 2.05; MeOH)

¹H-NMR (200 MHz, CDCl₃): δ 1.45-1.96 (m, 8H), 2.45-2.91 (m, 5H), 3.75 (dd, J=8.6, 6.5 Hz, 1H), 3.82 (s, 3H), 3.85 (s, 9H), 3.86 (s, 3H), 4.06 (dd, J=8.6, 6.5 Hz, 1H), 4.19 (dd, J=11.4, 7.0 Hz, 1H), 4.38 (dd, J=11.4, 6.7 Hz, 1H), 4.78 (d, J=6.1, 1 H), 6.53 (s, 2H), 6.64-6.84 (m, 3H).

¹³C-NMR (50 MHz, CDCl₃): δ 25.6, 29.9, 33.1, 42.5, 43.8, 49.1, 50.1, 55.8, 55.9, 60.8, 62.5, 72.8, 83.1, 102.5, 111.3, 111.8, 120.4, 128.6, 132.5, 138.2, 147.5, 148.9, 153.3, 176.5.

### Example 41

### 10-89: ((2S,3R,4R)-4-(3,4-dimethoxybenzyl)-2-(3,4,5-trimethoxyphenyl)tetrahydro-furan-3-yl)methyl cyclopropanecarboxylate (2637)

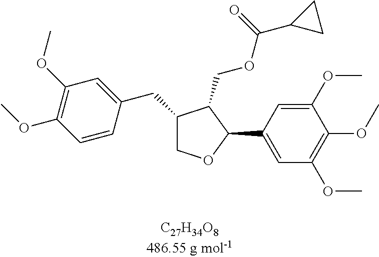

Preparation, work-up and purification identical to that of 10-85 except for using cyclopropanecarboxylic acid to give a slightly yellow, viscous oil (26.2 mg, 75%).

[α]_{D}²⁰: +20.4 (MeOH; c 1.785)

¹H-NMR (200 MHz, CDCl₃): δ 0.78-1.03 (m, 4H), 1.49-1.65 (m, 1H), 2.47-2.93 (m, 4H), 3.74 (dd, J=8.6, 6.5 Hz, 1H), 3.82 (s, 3H), 3.85 (s, 9H), 3.86 (s, 3H), 4.07 (dd, J=8.6, 6.2 Hz, 1H), 4.20 (dd, J=11.2, 7.2 Hz, 1H), 4.38 (dd, J=11.2, 6.8 Hz, 1H), 4.79 (d, J=6.1 Hz, 1H), 6.54 (s, 2H), 6.64-6.84 (m, 3H).

¹³C-NMR (50 MHz, CDCl₃): δ 8.5, 12.8, 33.1, 42.4, 49.0, 55.8, 56.1, 60.8, 62.7, 72.8, 83.2, 102.5, 111.3, 111.8, 120.4, 132.5, 138.2, 147.5, 148.9, 153.2, 174.7. One C_{q} not visible.

### Examples 42 and 43

In this final group of examples, free alcohols of formula (9) are transformed into compounds of formula (10) through Williamson etherification.

### Example 42

### 10-90: (2S,3R,4R)-3-((allyloxy)methyl)-4-(3,4-dimethoxybenzyl)-2-(3,4-dimethoxy-phenyl)tetrahydrofuran (2540)

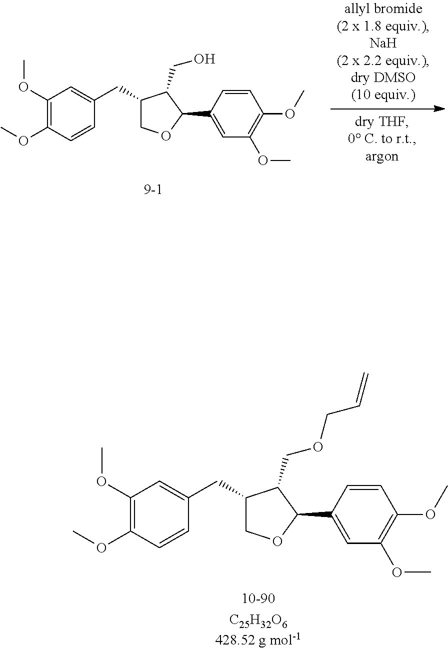

Preparation: a reaction vessel was charged with a stir bar, NaH mineral oil dispersion (approximately 60%, 8.8 mg, 0.22 mmol, 2.2 equiv.) and then evacuated and back-filled with argon using standard Schlenk technique (3×). Then was added dry THF (0.5 mL), followed by dry DMSO (71 μL, 1.0 mmol, 10 equiv.), both via syringe, and the stirred suspension cooled to 0° C. in an ice bath. This was followed by the dropwise addition of 9-1 (38.8 mg, 0.100 mmol, 1.0 equiv.) in dry THF (1.0 mL), subsequent stirring of the reaction for 15 min at 0° C., and finally by allyl bromide (16 μL, 0.18 mmol, 1.8 equiv.), both via syringe. The ice bath was removed and the mixture stirred for 18 h at room temperature, before the vessel was briefly opened to add more NaH dispersion (8.8 mg, 0.22 mmol, 2.2 equiv.). 1 h after that, more allyl bromide (16 μL, 0.18 mmol, 1.8 equiv.) was added via syringe, the mixture stirred for 25 h at room temperature.

Work-up and purification: the reaction was quenched by the addition of saturated aqueous NH₄Cl (1.0 mL) and extracted with Et₂O (2×10 mL). The combined organic phases were dried with Na₂SO₄, the solvents evaporated and the target compound purified by flash column chromatography (18 g silica, flow rate 20 mL/min, gradient 10 to 90% EtOAc in LP within 60 min) to give a nearly colorless oil (31.8 mg, 74%).

[α]_{D}²⁰: +19.6 (c 1.63, MeOH)

HR-MS (ESI) calc'd for M+Na⁺: 451.2091. found: 451.2096.

¹H-NMR (200 MHz, CDCl₃): δ 2.41-2.60 (m, 2H), 2.63-2.82 (m, 1H), 2.93 (dd, J=13.0, 4.2 Hz, 1H), 3.52 (dd, J=9.3, 6.3 Hz, 1H), 3.60-3.81 (m, 2H), 3.87 (s, 3H), 3.87 (s, 6H), 3.88 (s, 3H), 3.95-4.09 (m, 3H), 4.80 (d, J=6.6 Hz, 1H), 5.20 (ddt, J=10.3, 1.7, 1.3 Hz, 1H), 5.30 (ddt, J=17.3, 1.7, 1.6 Hz, 1H), 5.93 (dddd, J=17.3, 10.3, 5.9, 5.0 Hz, 1H), 6.67-6.92 (m, 6H).

¹³C-NMR (50 MHz, CDCl₃): δ 33.2, 42.7, 50.5, 56.0, 56.0, 56.0, 56.0, 68.2, 72.3, 72.9, 82.8, 109.1, 111.0, 111.3, 112.0, 117.1, 118.1, 120.6, 133.3, 134.8, 135.7, 147.5, 148.4, 149.0, 149.1.

### Example 43

### 10-91: (2S,3R,4R)-4-(3,4-dimethoxybenzyl)-2-(3,4-dimethoxyphenyl)-3-((prop-2-yn-1-yloxy)methyl)tetrahydrofuran (2541)

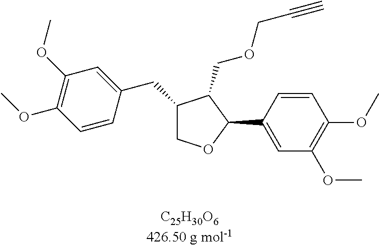

Preparation identical to that of 10-90 except for using propargyl bromide (2×20 μL, 2×0.18 mmol, 2×1.8 equiv.) in place of allyl bromide.

Work-up and purification: the reaction was quenched by the addition of saturated aqueous NH₄Cl (1.0 mL) and extracted with Et₂O (2×10 mL). The combined organic phases were dried with Na₂SO₄, the solvents evaporated and the target compound purified by flash column chromatography (18 g silica, flow rate 20 mL/min, gradient 10 to 90% EtOAc in LP within 60 min) to give a nearly colorless oil (23.2 mg, 54%).

[α]_{D}²⁰: +25.1 (c 1.49, MeOH)

HR-MS (ESI) calc'd for M+Na⁺: 449.1935. found: 449.1940.

¹H-NMR (200 MHz, CDCl₃): δ 2.41-2.61 (m, 2H), 2.45 (t, J=2.4 Hz, 1H), 2.62-2.83 (m, 1H), 2.95 (dd, J=13.0, 4.2 Hz, 1H), 3.60 (dd, J=9.1, 6.2 Hz, 1H), 3.81-3.69 (m, 2H), 3.86 (s, 3H), 3.87 (s, 6H), 3.89 (s, 3H), 4.03 (dd, J=8.5, 6.3 Hz, 1H), 4.18 (t, J=2.1 Hz, 2H), 4.80 (d, J=6.7 Hz, 1H), 6.92-6.66 (m, 6H).

### Example 44—Activity Measurements

The following assays were conducted to determine the cell proliferation-inhibiting activity of the test compounds produced as described above.

For determination of vascular SMC proliferation, 0.5×10⁴ viable rat aortic SMCs per well were seeded in SMC growth medium (DMEM/F12 medium with 20% serum, 30 μg/mL Gentamicin and 15 ng/mL Amphotericin) in 96-well plates. 24 h later, the medium was removed, the cells were washed once with SMC starvation medium (DMEM/F12 medium with 0.1% serum, 0.2% BSA, 30 μg/mL Gentamicin and 15 ng/mL Amphotericin) and incubated in starvation medium for further 24 h.

The quiescent cells were then pretreated for 30 min with the test compounds and induced to proliferate with 20 ng/mL platelet-derived growth factor (PDGF). Unstimulated cells were utilized for normalization and estimation of the basal level of proliferation. The final concentration of the solvent vehicle, dimethyl sulfoxide (DMSO), was the same (0.1%) in all wells. 48 h later, the SMC proliferation was quantified by resazurin dye conversion for 2 h as previously described (H. Joa et al., J. Nat. Prod. 74(6), 1513-6 (2011)).

For determination of endothelial cell proliferation, 0.5×10⁴ HUVECtert immortalized human umbilical vein endothelial cells (H. B. Schiller et al., Mol. Biol. Cell. 20(3), 745-756 (2009)) were seeded in 96-well plates for 24 h in HUVEC Complete Medium (EBM™ growth medium supplemented with 10% fetal bovine serum, EBM™ SingleQuots (Lonza, Basel, Switzerland), 100 U/mL benzylpenicillin, 100 μg/mL streptomycin, and 1% amphotericin). Afterwards, the medium was exchanged with fresh HUVEC Complete Medium and cells were treated with the indicated compounds for 48 h. After the end of the incubation period, the medium was removed, the cells were washed once with 200 μL/well PBS, and treated with 150 μL HUVEC Complete Medium containing 10 μg/mL resazurin. 2 h later, the fluorescence was measured at a wavelength of 580 nm, with excitation wavelength 535 nm in a 96-well plate reader (Tecan GENios Pro; Tecan Group Ltd., Mannedorf, Switzerland).

In every case, the percentage of viable cells was subtracted from 1, in order to obtain a measure of efficiency of the test compounds towards each particular type of cells, and subsequently, the efficiency quotient towards SMCs and ECs was calculated and taken as the “activity ratio” and thus as a measure of selective efficiency of each test compound towards SMCs in relation to ECs. For example, the synthetic Leoligin with the bio ID 2418 prepared in Comparative Example 27 as compound 10-22 gave the following results:

| | |
|---|---|
| Percentage of surviving SMCs: | 0.81 (81%) |
| Percentage of surviving ECs: | 0.51 (51%) |
| Efficiency towards SMCs: | 1-0.81 = 0.19 |
| Efficiency towards ECs: | 1-0.51 = 0.49 |
| Activity ratio: | 0.19/0.49 = 0.39 |

This ratio <1 shows that Leoligin was selectively inhibiting ECs relative to SMCs, i.e. with a factor of approximately 2.5 (inverse value of 0.39). As mentioned above, this result is consistent with previous assays performed at the University of Innsbruck, Austria, from which a ratio of IC₅₀ values of 0.33 can be derived.

Surprisingly, though, the inventors have found that some of the newly synthesized derivatives of Leoligin exhibit a selectivity which is precisely inverse, i.e. they selectively inhibit SMCs compared to ECs, which is especially preferred, for example, in treating or preventing restenosis, respectively.

Due to the usual fluctuations in determining surviving cells, which amount to approximately ±10%, and due to the relatively strong resulting fluctuations in calculating quotients—especially if the inhibitory effect of said novel compounds towards ECs is very poor—said threshold value of 1.50 for the activity ratio, as already mentioned above, was introduced. However, this does not preclude that compounds that to date did not show the desired activity, will do so in future and will as such be useful in the purpose of this invention, in which case they should be regarded as compounds comprised by the present invention.

In the following, a table listing every compound prepared in examples and comparative Examples for which an activity ratio (a.r.) of >1.0 was determined, is given. Due to the above-mentioned fluctuations, this list does not include ratios that were calculated accurately, but which were solely classified within the following four groups using letter-based designations:

| | |
|---|---|
| Comparative Examples - V: | 1.0 < a.r. < 1.50 |
| Examples of the Invention - A: | a.r. ≧ 1.50 |
| Preferred Examples - AA: | a.r. ≧ 2.0 |
| Especially preferred Examples - AAA: | a.r. ≧ 5.0 |

However, the order given in Table 1 does indeed correspond to the calculated values.

TABLE 1

| Example | Bio ID | a.r. code |
|---|---|---|
| Example 38 | 2632 | AAA |
| Example 13 | 3026 | AAA |
| Example 8 | 2760 | AAA |
| Example 18 | 3010 | AAA |
| Example 42 | 2540 | AAA |
| Example 15 | 3016 | AAA |
| Example 2 | 2754 | AAA |
| Example 19 | 2755 | AAA |
| Example 7 | 2756 | AAA |
| Example 16 | 3015 | AAA |
| Example 32 | 2862 | AAA |
| Example 17 | 2864 | AAA |
| Example 37 | 2629 | AAA |
| Example 34 | 2628 | AAA |
| Example 12 | 2823 | AAA |
| Example 23 | 2549 | AA |
| Example 9 | 2767 | AA |
| Example 6 | 3013 | AA |
| Example 33 | 2863 | AA |
| Example 40 | 2635 | AA |
| Example 41 | 2637 | AA |
| Example 3 | 2821 | AA |
| Example 5 | 3008 | AA |
| Example 27 | 2745 | AA |
| Example 10 | 2788 | AA |
| Example 21 | 2771 | AA |
| Example 20 | 2765 | AA |
| Example 25 | 2738 | AA |
| Example 22 | 2774 | AA |
| Example 35 | 2636 | AA |
| Example 24 | 2739 | AA |
| Example 30 | 2867 | AA |
| Example 28 | 2746 | A |
| Example 43 | 2541 | A |
| Example 4 | 3005 | A |
| Example 1 | 2749 | A |
| Example 26 | 2742 | A |
| Example 14 | 3029 | A |
| Example 29 | 2781 | A |
| Example 11 | 2792 | A |
| Example 36 | 2638 | A |
| Example 31 | 2856 | A |
| Example 39 | 2633 | A |
| Comparative Example 26 | 2548 | V |
| Comparative Example 34 | 2775 | V |
| Comparative Example 35 | 2539 | V |
| Comparative Example 13 | 2757 | V |
| Comparative Example 40 | 3027 | V |
| Comparative Example 43 | 2861 | V |
| Comparative Example 45 | 2734 | V |
| Comparative Example 33 | 2770 | V |
| Comparative Example 41 | 2854 | V |
| Comparative Example 24 | 2827 | V |
| Comparative Example 38 | 2784 | V |
| Comparative Example 29 | 2859 | V |
| Comparative Example 36 | 2544 | V |
| Comparative Example 42 | 2855 | V |
| Comparative Example 44 | 2860 | V |
| Comparative Example 46 | 2634 | V |
| Comparative Example 28 | 3030 | V |
| Comparative Example 31 | 2764 | V |
| Comparative Example 39 | 2866 | V |
| Comparative Example 30 | 3011 | V |
| Comparative Example 32 | 2769 | V |
| Comparative Example 37 | 2545 | V |

In any case, the embodiments given above clearly demonstrate that part (namely 43 out of 130 in total, i.e. approximately one third) of all novel compounds synthesized by the inventors are capable of selectively inhibiting SMC proliferation compared to EC proliferation. Therefore, the compounds are not only suitable for treating diseases and disorders caused by an excessive proliferation of SMCs, but also especially for treating or preventing restenosis after stent implantation procedures. Therefore, the invention also comprises appropriate drugs and pharmaceutical compositions as well as stents that were coated with it, respectively.

Moreover, a method for preparing these novel compounds is provided, in which said compounds can be prepared in notably higher yields, more cost-effectively and more quickly than the state of the art would previously allow for.

1: A compound of formula (II): 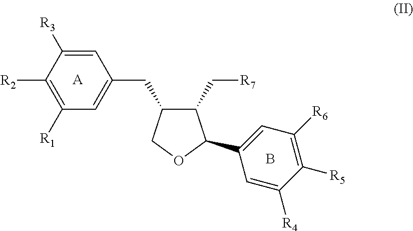
wherein, in Analog (IIa):
R₁ to R₆ are independently —H, —F, —CH₃, —CF₃, —CF₂CH₃, —OCH₃, —COCH₃, —C₄H₉, —COOC₂H₅, or —C₆H₅, or two vicinal residues from R₁ to R₆ form a saturated or unsaturated carbocyclic ring together with the two carbon atoms to which two vicinal residues are attached;
R₇ is OH, propargyloxy, cyclopropylcarbonyloxy, cyclobutylcarbonyl-oxy, cyclopentylcarbonyloxy, cyclopentenylcarbonyloxy, cyclohexylcarbonyloxy, cyclo-hexenylcarbonyloxy, adamantylethanoyloxy, 3-phenylpropenoyloxy (cinnamyloyloxy), 2-methylbenzoyloxy, and naphthoyloxy; and
wherein, in ring A and/or in ring B, one or more ring carbon atoms may be replaced by heteroatoms;
or, in Analog (IIb),
R₁ to R₆ are independently —H, —F, —CH₃, —OCH₃, or —C₄H₉;
R₇ is allyloxy, 2,2-dimethylpropanoyloxy (pivaloyloxy), butanoyloxy, 3-methylbutanoyloxy, 2-butenoyloxy, 2-methyl-2-butenoyloxy, 3-methyl-2-butenoyloxy, isopentanoyloxy, 2-ethylbutanoyloxy, or 3,3-dimethylbutanoyloxy;
wherein the Analog (IIa) is
((2S,3R,4R)-4-(4-(trifluoromethyl)benzyl)-2-(3,4,5-trimethoxyphenyl)tetrahydrofuran-3-yl)methanol (2760),
(Z)-2-methyl-2-butenoic acid ((2S,3R,4R)-2-(4-fluorophenyl)-4-(4-(trifluoromethyl)-benzyl)tetrahydrofuran-3-yl)methyl ester (3016),
((2S,3R,4R)-2-(3,4-dimethoxyphenyl)-4-(4-methylbenzyl)tetrahydrofuran-3-yl)-methanol (2754),
((2S,3R,4R)-4-benzyl-2-(3,4,5-trimethoxyphenyl)tetrahydrofuran-3-yl)methanol (2756),
(Z)-2-methyl-2-butenoic acid ((2S,3R,4R)-2-phenyl-4-(4-(trifluoromethyl)benzyl)-tetrahydrofuran-3-yl)methyl ester (3015),
(Z)-2-methyl-2-butenoic acid ((2S,3R,4R)-2-(3,4-dimethoxyphenyl)-4-(pyridine-2-yl-methyl)tetrahydrofuran-3-yl)methyl ester (2823),
((2S,3R,4R)-4-(biphenyl-4-ylmethyl)-2-(3,4,5-trimethoxyphenyl)tetrahydrofuran-3-yl)methanol (2767),
((2S,3R,4R)-4-(4-(1,1-difluoroethyl)benzyl)-2-(3,4-dimethoxyphenyl)tetrahydrofuran-3-yl)methanol (3013),
cyclobutanecarboxylic acid ((2S,3R,4R)-4-(3,4-dimethoxybenzyl)-2-(4-fluorophenyl)-tetrahydrofuran-3-yl)methyl ester (2863),
cyclopentanecarboxylic acid ((2S,3R,4R)-4-(3,4-dimethoxybenzyl)-2-(3,4,5-trimethoxyphenyl)tetrahydrofuran-3-yl)methyl ester (2635),
cyclopropanecarboxylic acid ((2S,3R,4R)-4-(3,4-dimethoxybenzyl)-2-(3,4,5-trimethoxyphenyl)tetrahydrofuran-3-yl)methyl ester (2637),
((2S,3R,4R)-2-(3,4-dimethoxyphenyl)-4-(naphthalene-1-ylmethyl)tetrahydrofuran-3-yl)methanol (2821),
((2S,3R,4R)-4-(4-butylbenzyl)-2-(3,4-dimethoxyphenyl)tetrahydrofuran-3-yl)methanol (3008),
2-(adamantane-1-yl)acetic acid ((2S,3R,4R)-4-(3,4-dimethoxybenzyl)-2-(3,4-dimethoxyphenyl)tetrahydrofuran-3-yl)methyl ester (2745),
(Z)-2-methyl-2-butenoic acid ((2S,3R,4R)-4-(4-acetyl benzyl)-2-(3,4,5-trimethoxy-phenyl)tetrahydrofuran-3-yl)methyl ester (2771),
4-(((3R,4R,5S)-4-((((Z)-2-methyl-2-butenoyl)oxy)methyl)-5-(3,4,5-trimethoxy-phenyl)tetrahydrofuran-3-yl)methyl)benzoic acid ethyl ester (2774),
cyclopentene-1-carboxylic acid ((2S,3R,4R)-4-(3,4-dimethoxybenzyl)-2-(3,4,5-trimethoxyphenyl)tetrahydrofuran-3-yl)methyl ester (2636),
cinnamic acid ((2S,3R,4R)-4-(3,4-dimethoxybenzyl)-2-(3,4-dimethoxyphenyl)-tetrahydrofuran-3-yl)methyl ester (2739),
cyclopentanecarboxylic acid ((2S,3R,4R)-4-(4-(tert-butyl)benzyl)-2-(3,4-dimethoxy-phenyl)tetrahydrofuran-3-yl)methyl ester (2867),
cyclohexanecarboxylic acid ((2S,3R,4R)-4-(3,4-dimethoxybenzyl)-2-phenyltetrahydrofuran-3-yl)methyl ester (2746),
(2S,3R,4R)-4-(3,4-dimethoxybenzyl)-2-(3,4-dimethoxyphenyl)-3-(propargyloxy-methyl)tetrahydrofuran (2541),
((2S,3R,4R)-2-(3,4-dimethoxyphenyl)-4-(4-(trifluoromethyl)benzyl)tetrahydrofuran-3-yl)methanol (3005),
((2S,3R,4R)-4-(4-(tert-butyl)benzyl)-2-(3,4-dimethoxyphenyl)tetrahydrofuran-3-yl)-methanol (2749),
cyclopentanecarboxylic acid ((2S,3R,4R)-4-(3,4-dimethoxybenzyl)-2-phenyl-tetrahydrofuran-3-yl)methyl ester (2742),
(Z)-2-methyl-2-butenoic acid ((2S,3R,4R)-4-(4-(1,1-difluoroethyl)benzyl)-2-(3,4-dimethoxyphenyl)tetrahydrofuran-3-yl)methyl ester (3029),
cyclopentanecarboxylic acid ((2S,3R,4R)-4-(3,4-dimethoxybenzyl)-2-(4-fluoro-phenyl)tetrahydrofuran-3-yl)methyl ester (2781),
4-(((3R,4R,5S)-5-(3,4-dimethoxyphenyl)-4-((((Z)-2-methyl-2-butenoyl)oxy)methyl)-tetrahydrofuran-3-yl)methyl)benzoic acid ethyl ester (2792),
benzoic acid ((2S,3R,4R)-4-(3,4-dimethoxybenzyl)-2-(3,4,5-trimethoxyphenyl)-tetrahydrofuran-3-yl)methyl ester (2638), or
cyclohexanecarboxylic acid ((2S,3R,4R)-4-(3,4-dimethoxybenzyl)-2-(3,4,5-trimethoxyphenyl)tetrahydrofuran-3-yl)methyl ester (2633); or
wherein the Analog (IIb) is:
isobutyric acid ((2S,3R,4R)-4-(3,4-dimethoxybenzyl)-2-(3,4,5-trimethoxyphenyl)-tetrahydrofuran-3-yl)methyl ester (2632),
(Z)-2-methyl-2-butenoic acid ((2S,3R,4R)-4-(4-butylbenzyl)-2-(3,4-dimethoxyphenyl)tetrahydrofuran-3-yl)methyl ester (3026),
(Z)-2-methyl-2-butenoic acid ((2S,3R,4R)-4-(4-(tert-butyl)benzyl)-2-(4-fluorophenyl)-tetrahydrofuran-3-yl)methyl ester (3010),
(2S,3R,4R)-3-((allyloxy)methyl)-4-(3,4-dimethoxybenzyl)-2-(3,4-dimethoxyphenyl)-tetrahydrofuran (2540),
3-methyl-2-butenoic acid ((2S,3R,4R)-4-(3,4-dimethoxybenzyl)-2-(3,4,5-trimethoxy-phenyl)tetrahydrofuran-3-yl)methyl ester (2755),
3-methylbutyric acid ((2S,3R,4R)-4-(3,4-dimethoxybenzyl)-2-(4-fluorophenyl)-tetrahydrofuran-3-yl)methyl ester (2862),
(Z)-2-methyl-2-butenoic acid ((2S,3R,4R)-4-(4-fluorobenzyl)-2-(4-fluorophenyl)-tetrahydrofuran-3-yl)methyl ester (2864),
butyric acid ((2S,3R,4R)-4-(3,4-dimethoxybenzyl)-2-(3,4,5-trimethoxyphenyl)-tetrahydrofuran-3-yl)methyl ester (2629),
(E)-2-methyl-2-butenoic acid ((2S,3R,4R)-4-(3,4-dimethoxybenzyl)-2-(3,4,5-trimethoxyphenyl)tetrahydrofuran-3-yl)methyl ester (2628),
(E)-2-butenoic acid ((2S,3R,4R)-4-(3,4-dimethoxybenzyl)-2-(3,4-dimethoxyphenyl)-tetrahydrofuran-3-yl)methyl ester (2549),
(Z)-2-methyl-2-butenoic acid ((2S,3R,4R)-4-(4-(tert-butyl)benzyl)-2-(3,4-dimethoxy-phenyl)tetrahydrofuran-3-yl)methyl ester (2788),
(Z)-2-methyl-2-butenoic acid ((2S,3R,4R)-4-(4-(tert-butyl)benzyl)-2-(3,4,5-trimethoxyphenyl)tetrahydrofuran-3-yl)methyl ester (2765),
3-methyl-2-butenoic acid ((2S,3R,4R)-4-(3,4-dimethoxybenzyl)-2-(3,4-dimethoxy-phenyl)tetrahydrofuran-3-yl)methyl ester (2738), or
2-ethylbutyric acid ((2S,3R,4R)-4-(3,4-dimethoxybenzyl)-2-(4-fluorophenyl)-tetrahydrofuran-3-yl)methyl ester (2856), and
wherein the compound of formula (II) selectively inhibits smooth muscle cell (SMC) proliferation to a higher extent than endothelial cell (EC) proliferation.

2. (canceled)

3: The compound of claim 1, which is
isobutyric acid ((2S,3R,4R)-4-(3,4-dimethoxybenzyl)-2-(3,4,5-trimethoxyphenyl)-tetrahydrofuran-3-yl)methyl ester (2632),
(Z)-2-methyl-2-butenoic acid ((2S,3R,4R)-4-(4-butyl benzyl)-2-(3,4-dimethoxyphenyl)tetrahydrofuran-3-yl)methyl ester (3026),
((2S,3R,4R)-4-(4-(trifluoromethyl)benzyl)-2-(3,4,5-trimethoxyphenyl)tetrahydrofuran-3-yl)methanol (2760),
(Z)-2-methyl-2-butenoic acid ((2S,3R,4R)-4-(4-(tert-butyl)benzyl)-2-(4-fluorophenyl)-tetrahydrofuran-3-yl)methyl ester (3010),
(2S,3R,4R)-3-((allyloxy)methyl)-4-(3,4-dimethoxybenzyl)-2-(3,4-dimethoxyphenyl)-tetrahydrofuran (2540),
(Z)-2-methyl-2-butenoic acid ((2S,3R,4R)-2-(4-fluorophenyl)-4-(4-(trifluoromethyl)-benzyl)tetrahydrofuran-3-yl)methyl ester (3016),
((2S,3R,4R)-2-(3,4-dimethoxyphenyl)-4-(4-methylbenzyl)tetrahydrofuran-3-yl)-methanol (2754),
3-methyl-2-butenoic acid ((2S,3R,4R)-4-(3,4-dimethoxybenzyl)-2-(3,4,5-trimethoxy-phenyl)tetrahydrofuran-3-yl)methyl ester (2755),
((2S,3R,4R)-4-benzyl-2-(3,4,5-trimethoxyphenyl)tetrahydrofuran-3-yl)methanol (2756),
(Z)-2-methyl-2-butenoic acid ((2S,3R,4R)-2-phenyl-4-(4-(trifluoromethyl)benzyl)-tetrahydrofuran-3-yl)methyl ester (3015),
3-methylbutenoic acid ((2S,3R,4R)-4-(3,4-dimethoxybenzyl)-2-(4-fluorophenyl)-tetrahydrofuran-3-yl)methyl ester (2862),
(Z)-2-methyl-2-butenoic acid ((2S,3R,4R)-4-(4-fluorobenzyl)-2-(4-fluorophenyl)-tetrahydrofuran-3-yl)methyl ester (2864),
butyric acid ((2S,3R,4R)-4-(3,4-dimethoxybenzyl)-2-(3,4,5-trimethoxyphenyl)-tetrahydrofuran-3-yl)methyl ester (2629),
(E)-2-methyl-2-butenoic acid ((2S,3R,4R)-4-(3,4-dimethoxybenzyl)-2-(3,4,5-trimethoxyphenyl)tetrahydrofuran-3-yl)methyl ester (2628),
(Z)-2-methyl-2-butenoic acid ((2S,3R,4R)-2-(3,4-dimethoxyphenyl)-4-(pyridine-2-yl-methyl)tetrahydrofuran-3-yl)methyl ester (2823),
(E)-2-butenoic acid ((2S,3R,4R)-4-(3,4-dimethoxybenzyl)-2-(3,4-dimethoxyphenyl)-tetrahydrofuran-3-yl)methyl ester (2549),
((2S,3R,4R)-4-(biphenyl-4-yl-methyl)-2-(3,4,5-trimethoxyphenyl)tetrahydrofuran-3-yl)methanol (2767),
((2S,3R,4R)-4-(4-(1,1-difluoroethyl)benzyl)-2-(3,4-dimethoxyphenyl)tetrahydrofuran-3-yl)methanol (3013),
cyclobutanecarboxylic acid ((2S,3R,4R)-4-(3,4-dimethoxybenzyl)-2-(4-fluorophenyl)-tetrahydrofuran-3-yl)methyl ester (2863),
cyclopentanecarboxylic acid ((2S,3R,4R)-4-(3,4-dimethoxybenzyl)-2-(3,4,5-trimethoxyphenyl)tetrahydrofuran-3-yl)methyl ester (2635),
cyclopropanecarboxylic acid ((2S,3R,4R)-4-(3,4-dimethoxybenzyl)-2-(3,4,5-trimethoxyphenyl)tetrahydrofuran-3-yl)methyl ester (2637),
((2S,3R,4R)-2-(3,4-dimethoxyphenyl)-4-(naphthalene-1-ylmethyl)tetrahydrofuran-3-yl)methanol (2821),
((2S,3R,4R)-4-(4-butylbenzyl)-2-(3,4-dimethoxyphenyl)tetrahydrofuran-3-yl)methanol (3008),
2-(adamantane-1-yl)acetic acid ((2S,3R,4R)-4-(3,4-dimethoxybenzyl)-2-(3,4-dimethoxyphenyl)tetrahydrofuran-3-yl)methyl ester (2745),
(Z)-2-methyl-2-butenoic acid ((2S,3R,4R)-4-(4-(tert-butyl)benzyl)-2-(3,4-dimethoxy-phenyl)tetrahydrofuran-3-yl)methyl ester (2788),
(Z)-2-methyl-2-butenoic acid ((2S,3R,4R)-4-(4-acetyl benzyl)-2-(3,4,5-trimethoxy-phenyl)tetrahydrofuran-3-yl)methyl ester (2771),
(Z)-2-methyl-2-butenoic acid ((2S,3R,4R)-4-(4-(tert-butyl)benzyl)-2-(3,4,5-trimethoxyphenyl)tetrahydrofuran-3-yl)methyl ester (2765),
3-methyl-2-butenoic acid ((2S,3R,4R)-4-(3,4-dimethoxybenzyl)-2-(3,4-dimethoxy-phenyl)tetrahydrofuran-3-yl)methyl ester (2738),
4-(((3R,4R,5S)-4-((((Z)-2-methyl-2-butenoyl)oxy)methyl)-5-(3,4,5-trimethoxy-phenyl)tetrahydrofuran-3-yl)methyl)benzoic acid ethyl ester (2774),
cyclopentene-1-carboxylic acid ((2S,3R,4R)-4-(3,4-dimethoxybenzyl)-2-(3,4,5-trimethoxyphenyl)tetrahydrofuran-3-yl)methyl ester (2636),
cinnamic acid ((2S,3R,4R)-4-(3,4-dimethoxybenzyl)-2-(3,4-dimethoxyphenyl)-tetrahydrofuran-3-yl)methyl ester (2739), or
cyclopentanecarboxylic acid ((2S,3R,4R)-4-(4-(tert-butyl)benzyl)-2-(3,4-dimethoxy-phenyl)tetrahydrofuran-3-yl)methyl ester (2867).

4: The compound of claim 1, which is
isobutyric acid ((2S,3R,4R)-4-(3,4-dimethoxybenzyl)-2-(3,4,5-trimethoxyphenyl)-tetrahydrofuran-3-yl)methyl ester (2632),
(Z)-2-methyl-2-butenoic acid ((2S,3R,4R)-4-(4-butyl benzyl)-2-(3,4-dimethoxyphenyl)tetrahydrofuran-3-yl)methyl ester (3026),
((2S,3R,4R)-4-(4-(trifluoromethyl)benzyl)-2-(3,4,5-trimethoxyphenyl)tetrahydrofuran-3-yl)methanol (2760),
(Z)-2-methyl-2-butenoic acid ((2S,3R,4R)-4-(4-(tert-butyl)benzyl)-2-(4-fluorophenyl)-tetrahydrofuran-3-yl)methyl ester (3010),
(2S,3R,4R)-3-((allyloxy)methyl)-4-(3,4-dimethoxybenzyl)-2-(3,4-dimethoxyphenyl)-tetrahydrofuran (2540),
(Z)-2-methyl-2-butenoic acid ((2S,3R,4R)-2-(4-fluorophenyl)-4-(4-(trifluoromethyl)-benzyl)tetrahydrofuran-3-yl)methyl ester (3016),
((2S,3R,4R)-2-(3,4-dimethoxyphenyl)-4-(4-methylbenzyl)tetrahydrofuran-3-yl)-methanol (2754),
3-methyl-2-butenoic acid ((2S,3R,4R)-4-(3,4-dimethoxybenzyl)-2-(3,4,5-trimethoxy-phenyl)tetrahydrofuran-3-yl)methyl ester (2755),
((2S,3R,4R)-4-benzyl-2-(3,4,5-trimethoxyphenyl)tetrahydrofuran-3-yl)methanol (2756),
(Z)-2-methyl-2-butenoic acid ((2S,3R,4R)-2-phenyl-4-(4-(trifluoromethyl)benzyl)-tetrahydrofuran-3-yl)methyl ester (3015),
3-methylbutenoic acid ((2S,3R,4R)-4-(3,4-dimethoxybenzyl)-2-(4-fluorophenyl)-tetrahydrofuran-3-yl)methyl ester (2862),
(Z)-2-methyl-2-butenoic acid ((2S,3R,4R)-4-(4-fluorobenzyl)-2-(4-fluorophenyl)-tetrahydrofuran-3-yl)methyl ester (2864),
butyric acid ((2S,3R,4R)-4-(3,4-dimethoxybenzyl)-2-(3,4,5-trimethoxyphenyl)-tetrahydrofuran-3-yl)methyl ester (2629),
(E)-2-methyl-2-butenoic acid ((2S,3R,4R)-4-(3,4-dimethoxybenzyl)-2-(3,4,5-trimethoxyphenyl)tetrahydrofuran-3-yl)methyl ester (2628), or
(Z)-2-methyl-2-butenoic acid ((2S,3R,4R)-2-(3,4-dimethoxyphenyl)-4-(pyridine-2-yl-methyl)tetrahydrofuran-3-yl)methyl ester (2823).

5. (canceled)

6: A stent or another other implant comprising:
the compound of claim 1.

7: The stent or other implant of claim 6, which is a tissue, an organ, or part of an organ.

8: A pharmaceutical composition, comprising:
the compound of claim 1; and
a pharmaceutically acceptable carrier or excipient.

9: A method of selectively inhibiting smooth muscle cell (SMC) proliferation compared to endothelial cell (EC) proliferation in vitro, the method comprising:
contacting a composition comprising a smooth muscle cell and an endothelial cell with the compound of claim 1.

10: A method for coating a body implant by applying a drug onto at least one surface of the implant, the method comprising:
applying to the implant the compound of claim 1.

11: The method of claim 10, wherein the implant is a stent.

12. (canceled)

13: A method of preparing the compound of claim 1, the method comprising:
a) optionally, reacting a benzaldehyde of formula (I) with vinyl magnesium bromide, or an alternative vinyl nucleophile, to obtain a racemic α-vinyl benzyl alcohol rac-(2) 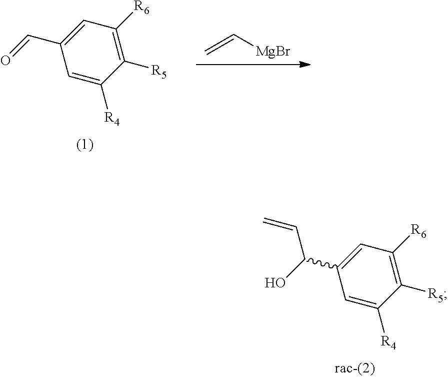
b) kinetically resolving the rac-(2) using enzymatic catalysis to obtain an (S)-enantiomer (S)-(2) and simultaneously form (R)—R₈-(2) as a by-product to be separated 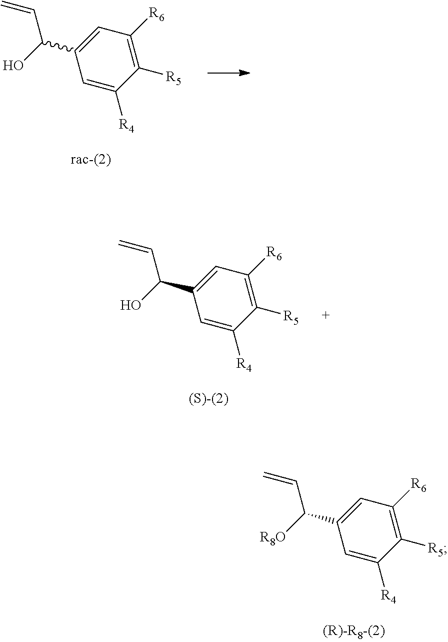
c) subsequently, in arbitrary order, either c1 esterifying the (S)-enantiomer (S)-(2) a propargylating agent, to give a propargyl ether (3) 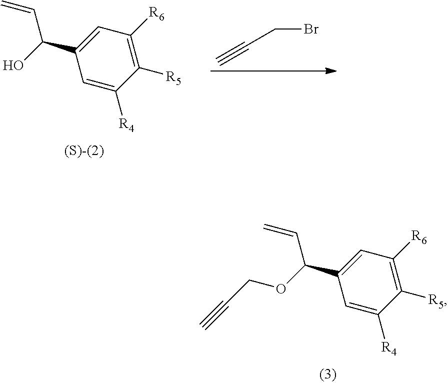
and subsequently oxidizing the propargyl ether (3) to give a propargyl ether epoxide (5) 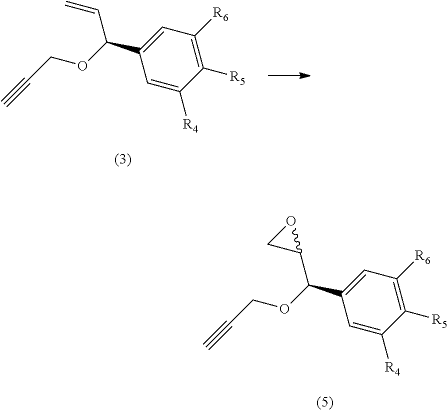
or, in reverse order, c2) oxidizing the (S)-enantiomer (S)-(2) to give an epoxide (4) 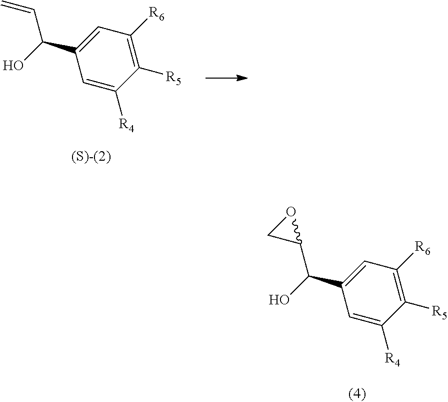
and, subsequently, esterifying the epoxide (4) with a propargylating agent, to give an propargyl ether epoxide (5) 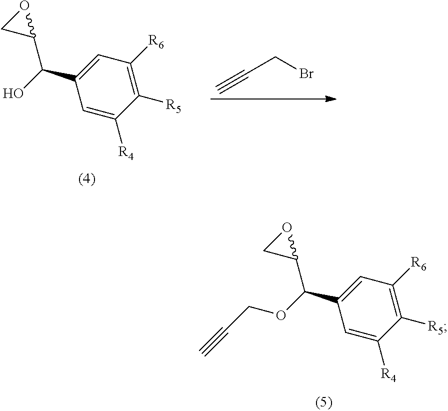
d) cyclizing the propargyl ether epoxide (5) using a cyclization agent to give a tetrahydrofuranyl methanol (6) 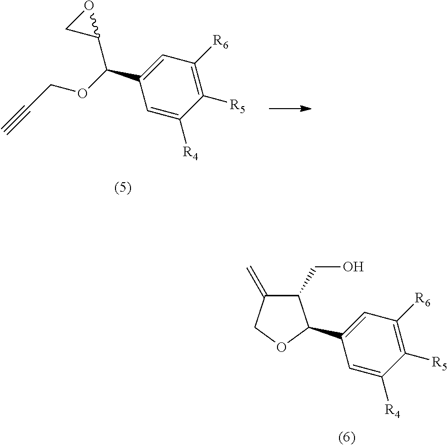
(e) protecting the alcohol functionality of tetrahydrofuranyl methanol (6) using a bulky protecting group, to give a protected methanol (7) 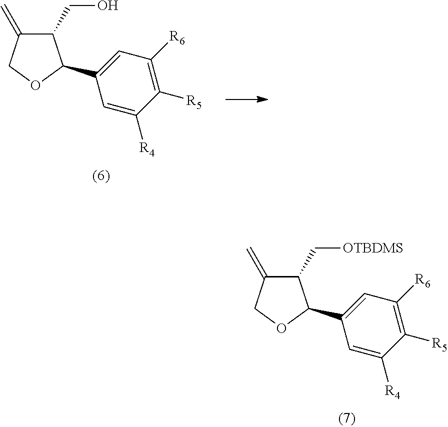
f) introducing a phenyl or 2-pyridyl residue (7b), substituted with corresponding residues R₁ to R₃, by reaction with the exocyclic methylene group of the protected methanol (7), to give a protected product (8) 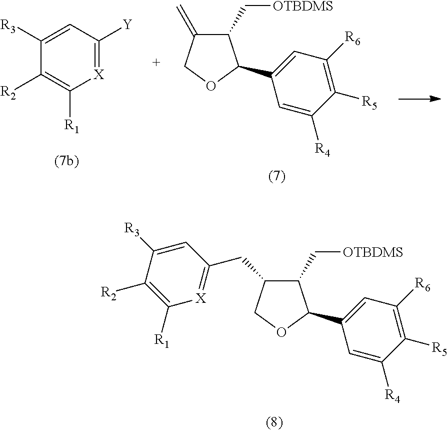
wherein, in each case, X is CH or N and Y is a leaving group;
g) cleaving the protecting group of the protected product (8) to give a free alcohol (9) 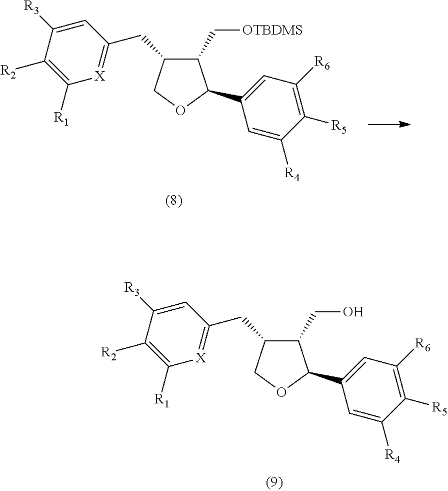
h) if R₇≠OH, simultaneously or subsequently etherifying or esterifying the free OH group, in order to obtain compound (10) comprising the corresponding residue R₇

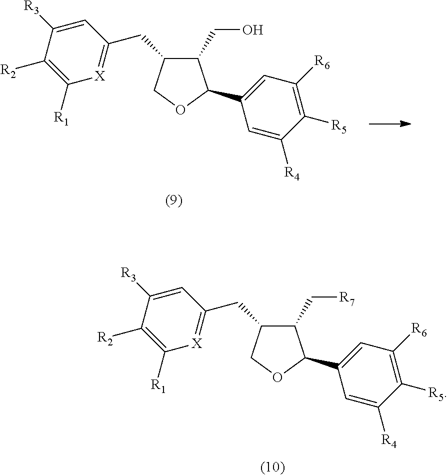

14: The method of claim 13, comprising, in b), using an enzyme agent comprising Amano Lipase PS used as the enzyme and using a donor component comprising vinyl acetate, so that residue R₈ is an acetyl residue and (R)-acetyl-(2) is produced as a by-product.

15: The method of claim 13, wherein the oxidizing in c1) after the propargylation, comprises using an oxidant comprising m-chloro-benzoic acid.

16: The method according to claim 13, wherein the oxidizing in c2), prior to propargylation, comprises an enantioselective Sharpless epoxidation using a co-reagent comprising (S,S)-diethyltartrate ((−)-DET), and an oxidant comprising tert-butylhydroperoxide (TBHP), and a catalyst comprising titanium(IV)-isopropanolate (Ti(O-i-Pr)₄).

17: The method of claim 13, wherein the cyclizing in d) comprises using a cyclization agent comprising titanocenedichloride (dichlorobis-(cyclopentadienyl)-titanium(IV) (CₚTiCl₂).

18: The method of claim 13, comprising, if R₇≠OH, esterifying the OH group with a respective acyl residue, so as to form a respective residue R₇ together with the oxygen atom, and omitting g) and h).

19: The method of claim 13, wherein comprises f) comprises performing a Suzuki coupling comprising, first, stereoselectively hydroborating, using an boration agent comprising an organoboran, the exocyclic methylene group of the protected methanol (7), to give intermediate (7a), then, immediately reacting the intermediate (7a) with the phenyl or 2-pyridyl residue (7b), wherein X is a halogen atom, thereby substituting the boryl residue with phenyl or 2-pyridyl residue substituted with R₁ to R₃.

20: The method of claim 19, comprising, in f1), reacting the protected methanol (7) with 9-borabicyclo[3.3.1]nonane, 9-BBN, to give a borane intermediate (7a) 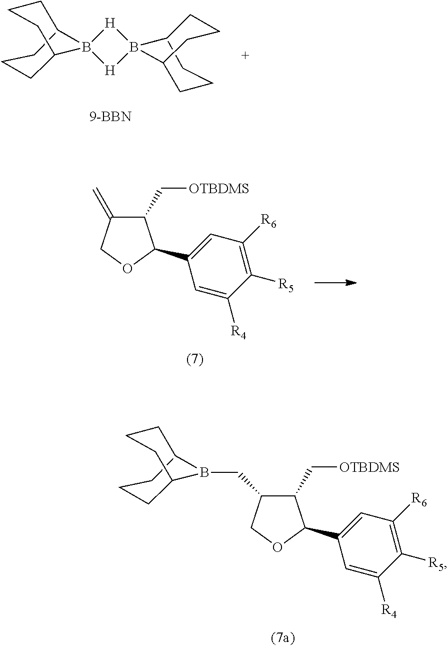
then, immediately following f2), reacting the borane intermediate (7a) with an appropriately substituted phenyl or 2-pyridyl bromide (7b) using a catalyst comprising 1,1′-bis(diphenylphosphino)ferrocene-dichloropalladium(II)-dichloromethane complex (Pd(dppf)Cl₂.CH₂Cl₂), to give the protected product (8).

21: The method of claim 13, comprising, in g), cleaving the protecting group is performed using tetra-n-butyl ammoniumfluoride (TBAF).

22: The method of claim 13, wherein f) and g) are conducted as one-pot reactions.

23: The method of claim 13, comprising, in e) or h), esterifying the OH group, by a Mitsunobu esterification or Steglich esterification, wherein a free acid serves as the protecting group and/or forms a respective residue R₇.

24: The method of claim 23, wherein the Mitsunobu esterification is conducted in the presence of 1,1′-(azodicarbonyl)dipiperidine (ADD), or diethylazodicarboxylate (DEAD), and tri-phenylphosphine (PPh₃).

25: The method of claim 23, the Steglich esterification is conducted in the presence of N-(3-dimethylaminopropyl)-N′-ethylcarbodiimide (EDC), or N,N′-dicyclohexylcarbodiimide (DCC), and 4-dimethylaminopyridine (DMAP).

26: The composition of claim 8, which is suitable for treating or preventing hyperplasia.
